(19) United States
(12) Patent Application Publication
(10) Pub. No.: US 2026/0166013 A1
(43) Pub. Date: Jun. 18, 2026

(54) METHODS OF IDENTIFYING SENOTHERAPEUTIC TARGETS AND TREATING MUSCLE DEGRADATION

(71) Applicant: Center for Neuromusculoskeletal Restorative Medicine Limited, Hong Kong (CN)

(72) Inventors: Huating Wang, Hong Kong (CN); Yang Li, Hong Kong (CN); Chuhan Li, Hong Kong (CN)

(21) Appl. No.: 19/201,112

(22) Filed: May 7, 2025

Related U.S. Application Data
(60) Provisional application No. 63/734,323, filed on Dec. 16, 2024.

(51) Int. Cl. A61K 31/4196; A61K 9/00; A61K 31/423; A61K 45/06; A61P 21/00; C12Q 1/6888; G01N 33/50

(52) U.S. Cl. CPC A61K 31/4196; A61K 9/0019; A61K 31/423; A61K 45/06; A61P 21/00; C12Q 1/6888; G01N 33/5023; G01N 33/5091; C12Q 2600/106; C12Q 2600/148; G01N 2500/10; G01N 2800/52

(57) ABSTRACT

A method for identifying senotherapeutic targets includes the steps of obtaining multiomics data from senescent cells to provide a senescence atlas, utilising the senescence atlas to identify molecular markers associated with cellular senescence, comparing the identified molecular markers with senescence-associated secretory phenotypes (SASPs) and senescence biomarkers, and identifying potential senotherapeutic targets involved in tissue degradation.

### TECHNICAL FIELD

This invention relates to a method for identifying senotherapeutic targets. In particular, the present disclosure is directed to a method for identifying senotherapeutic targets by providing a senescence atlas. The invention further relates to a method of treating muscle degradation and the use of compounds in the manufacture of a medicament for the treatment of muscle degradation.

### BACKGROUND

Ageing represents a natural and intricate progression marked by the gradual decline in physiological functions and an increased susceptibility to diseases. Concurrently, sarcopenia emerges as a distinct condition within the ageing spectrum, signifying the age-related degeneration of muscle mass, strength, and functionality. This phenomenon stems from a combination of factors including hormonal fluctuations, diminished physical activity, inadequate nutrition, chronic inflammation, and impaired muscle protein synthesis. The consequences of sarcopenia are profound, encompassing reduced mobility, heightened risks of falls and fractures, loss of independence, and an overall decline in quality of life for ageing individuals. Cellular senescence is a key feature of ageing and is a state in which cells stop dividing and enter a permanent growth arrest, while remaining metabolically active. Cellular senescence, an adaptive response induced by multiple physiological and pathological stresses that entails irreversible cell cycle arrest and resistance to apoptosis. The process can be triggered by various factors such as DNA damage, telomere shortening, or oncogene activation. Senescent cells are typically characterised by features such as size and irregularity of shape (altered morphology), Senescence-Associated β-Galactosidase (SA-β-gal) which is an enzyme that accumulates in these cells, and Senescence-Associated Secretory Phenotype (SASP), including secretion of pro-inflammatory cytokines, growth factors, and proteases that can affect neighbouring cells and the tissue microenvironment.

Cellular senescence has many functions and implications, including in development, tissue repair, cancer and ageing. For example, senescent cells can be beneficial for wound healing and tissue regeneration. Senescence also has an important role in tumour suppression and prevents damaged cells from proliferation. In ageing, however, the accumulation of senescent cells is detrimental and can contribute to ageing and age-related diseases by disrupting tissue function and promoting inflammation.

A better understanding of cellular senescence is crucial for identifying senotherapeutic targets for exploitation, and for the development of therapeutic strategies to target age-related disease, improve muscle function and reduce functional decline.

### SUMMARY OF THE INVENTION

Cellular senescence is a hallmark of organismal ageing and is characterised and driven by cellular and molecular mechanisms. Cellular senescence, an adaptive response induced by multiple physiological and pathological stresses that entails irreversible cell cycle arrest and resistance to apoptosis is a dominant mechanism orchestrating various facets of ageing physiology and development of many age-dependent diseases. The claimed methods of the invention advantageously provide a senescence atlas for the identification of novel senotherapeutic targets and a method of treating muscle degradation in a subject.

According to a first aspect of the invention, there is provided a method for identifying senotherapeutic targets, comprising the steps of:
obtaining multiomics data from senescent cells to provide a senescence atlas,
utilising the senescence atlas to identify molecular markers associated with cellular senescence,
comparing the identified molecular markets with senescence-associated secretory phenotypes (SASPs) and senescence biomarkers, and
identifying potential senotherapeutic targets involved in tissue degradation.

For example, the molecular markers include one or more of genes, proteins, epigenetic modifications, or secretory factors associated with senescence. In another example, the multiomics data includes one or more transcriptomic, epigenomic, or proteomic profiles. A detailed understanding of the transcriptomic and epigenomic heterogeneity of senescent cells in ageing human muscles will contribute to a better grasp of the biological functions of these cells, and also facilitate the identification of new markers and therapeutic strategies for alleviating sarcopenia and promoting healthy ageing.

In an example embodiment, the multiomics data is obtained using single-nucleus sequencing or other single-cell omics technologies.

In one example embodiment, the method further comprises a step of validating the identified potential senotherapeutic targets by assessing the role in cellular senescence in vitro or in vivo.

The method further includes a step of determining the effects of targeting the potential senotherapeutic targets on cellular senescence.

In one example embodiment, the potential senotherapeutic targets include genes, proteins, or signalling pathways involved in one or more of cellular senescence, inflammation, muscular degradation, sarcopenia, or age-related diseases.

In a further example embodiment, the senescence atlas is used to compare senescent and non-senescent cell profiles to identify specific alterations associated with senescence.

According to another aspect of the invention, there is provided a method of treating muscle degradation in a subject, the method comprising administering to the subject a composition comprising a therapeutically effective amount of a compound of Formula (I), or a pharmaceutically acceptable salt, solvate, or hydrate thereof:

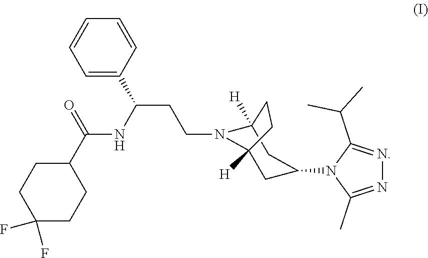 [SMILES: Cc1nnc(C(C)C)n1[C@@H]1C[C@H]2CC[C@@H](C1)N2CC[C@H](NC(=O)C1CCC(F)(F)CC1)c1ccccc1]

For example, the composition includes one or more pharmaceutically acceptable carriers or excipients.

In one example embodiment, the composition reduces or delays cellular senescence in the subject.

In a further example embodiment, the composition targets cellular senescence mechanisms to mitigate muscle degradation and improve muscle function in the subject. For example, the composition targets muscle atrophy or other related mechanisms to improve muscle function and reduce functional decline.

In one embodiment, the composition targets senescence-associated secretory phenotypes (SASPs) to mitigate muscle degradation and improve muscle function in the subject. For example, the composition targets one or more senotherapeutic targets.

In a preferred embodiment, the composition targets one or more senotherapeutic targets including genes, proteins, or signalling pathways involved in one or more of cellular senescence, inflammation, muscular degradation, sarcopenia, neurodegenerative diseases, cardiovascular ageing, or age-related diseases. In an example, the composition targets senescence biomarkers to mitigate muscle degradation and improve muscle function in the subject.

In an example embodiment, the therapeutically effective amount of the compound of Formula (I) administered is determined based on an assessment of degree of the muscle degradation in the subject and a severity of cellular senescence in the subject. For example, the muscle degradation is associated with sarcopenia, muscle wasting disorders, age-related muscular weakness, or a combination thereof. In a preferred example, the muscle degradation is associated with sarcopenia.

In one example embodiment, the subject is an elderly individual or subject diagnosed with muscle loss, for example age-related muscle loss.

In a further example embodiment, the composition is formulated for administration in the subject via oral, injectable, or topical routes. The method further comprises administering the composition in combination with one or more therapeutic agents or supplementary treatments targeting muscle regeneration or senescence. For example, the therapeutic agents include muscle growth stimulants, anti-inflammatory agents, or other senotherapeutic compounds, or a combination thereof.

The additional treatment of administering the composition in combination with one or more therapeutic agents improves the efficacy of the treatment.

According to a further aspect of the invention, there is provided use of a compound of Formula (I), or a pharmaceutically acceptable salt, solvate, or hydrate thereof, in the manufacture of a medicament for the treatment of muscle degradation in a subject:

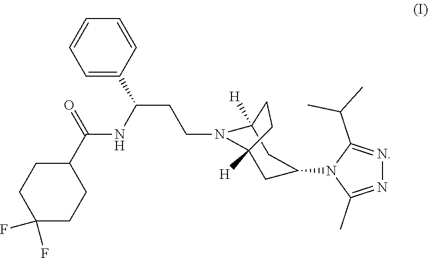 [SMILES: Cc1nnc(C(C)C)n1[C@@H]1C[C@H]2CC[C@@H](C1)N2CC[C@H](NC(=O)C1CCC(F)(F)CC1)c1ccccc1]

In a preferred embodiment, the medicament further comprises one or more pharmaceutically acceptable carriers or excipients.

For example, the medicament reduces or delays cellular senescence.

In an example embodiment, a therapeutically effective amount of the medicament is administered to mitigate muscle degradation and improve muscle function.

In one embodiment, the medicament is formulated to modulate the function of senescence-associated secretory phenotypes (SASPs) in muscle cells.

Preferably, the medicament targets one or more senotherapeutic targets including genes, proteins, or signalling pathways involved in one or more of cellular senescence, inflammation, muscular degradation, sarcopenia, neurodegenerative diseases, cardiovascular ageing, or age-related diseases.

For example, the medicament is formulated as a dosage form selected from the group consisting of tablets, capsules, and powders. In an embodiment, the medicament is formulated to deliver the compound of formula (I) in an extended-release or controlled-release dosage form.

In an example embodiment, the muscle degradation is associated with sarcopenia, muscle wasting disorders, age-related muscular weakness, or a combination thereof.

Most preferably, the muscle degradation is associated with sarcopenia.

According to a further aspect of the invention, there is provided a method of treating muscle degradation in a subject, the method comprising administering to the subject a composition comprising a therapeutically effective amount of a compound of Formula (II), or a pharmaceutically acceptable salt, solvate, or hydrate thereof:

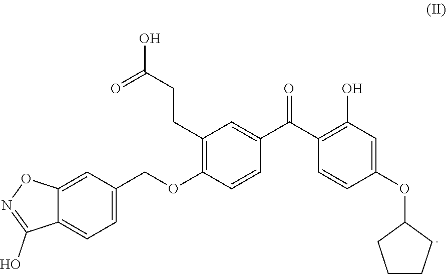 [SMILES: O=C(O)CCc1cc(C(=O)c2ccc(OC3CCCC3)cc2O)ccc1OCc1ccc2c(O)noc2c1]

The composition targets cellular senescence mechanisms to mitigate muscle degradation and improve muscle function in the subject.

In an example embodiment, the composition targets c-Fos/activator protein (AP)-1 family. For example, the composition targets one or more of JUNB, FOSL1, ATF6, FOSB, and JUND, resulting in delayed senescence. In a further example, the compound of Formula (II) is a small molecule inhibitor of c-Fos/AP-1 family including and blocks the function of JUNB. In an example embodiment, the composition targets JUNB and decreases CXCL1 to delay cellular senescence. In another example embodiment, the composition decreases CXCL1 expression and delays senescence. The composition advantageously targets the AP-1 family as a senomorphic approach. Most preferably, the composition targets JUNB to delay cellular senescence.

In an example embodiment, the composition is administered in combination with a therapeutically effective amount of a compound of Formula (I), or a pharmaceutically acceptable salt, solvate, or hydrate thereof:

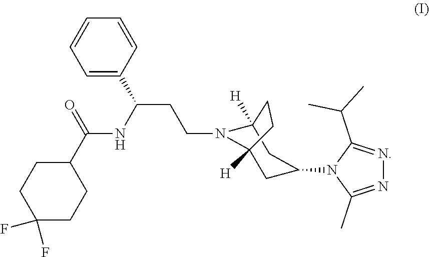 [SMILES: Cc1nnc(C(C)C)n1[C@@H]1C[C@H]2CC[C@@H](C1)N2CC[C@H](NC(=O)C1CCC(F)(F)CC1)c1ccccc1]

For example, the therapeutically effective amount of the compound of Formula (II) administered is determined based on an assessment of degree of the muscle degradation in the subject and a severity of cellular senescence in the subject. The muscle degradation is associated with sarcopenia, muscle wasting disorders, age-related muscular weakness, or a combination thereof. Further, the composition is beneficially formulated for administration in the subject via oral, injectable, or topical routes, and the composition may be administered in combination with one or more therapeutic agents or supplementary treatments targeting muscle regeneration or senescence, including muscle growth stimulants, anti-inflammatory agents, other senotherapeutic compounds, or a combination thereof.

A further aspect of the invention is directed to the use of a compound of Formula (II), or a pharmaceutically acceptable salt, solvate, or hydrate thereof, in the manufacture of a medicament for the treatment of muscle degradation in a subject:

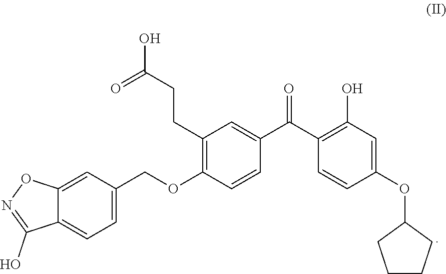 [SMILES: O=C(O)CCc1cc(C(=O)c2ccc(OC3CCCC3)cc2O)ccc1OCc1ccc2c(O)noc2c1]

In an example embodiment, the medicament advantageously reduces or delays cellular senescence. For example, a therapeutically effective amount of the medicament is administered to mitigate muscle degradation and improve muscle function. For example, the muscle degradation is associated with sarcopenia, muscle wasting disorders, age-related muscular weakness, or a combination thereof.

In a preferred embodiment, the medicament targets c-Fos/activator protein (AP)-1 family, and is formulated to modulate JUNB, FOSL1, ATF6, FOSB, and JUND in muscle cells resulting in reduced or delayed senescence in the muscle cells. Most preferably, the medicament targets JUNB to delay cellular senescence.

In an example embodiment, the medicament targets genes, proteins, or signalling pathways involved in one or more of cellular senescence, inflammation, muscular degradation, sarcopenia, neurodegenerative diseases, cardiovascular ageing, or age-related diseases.

In an alternative embodiment, the medicament further comprises a therapeutically effective amount of a compound of Formula (1), or a pharmaceutically acceptable salt, solvate, or hydrate thereof:

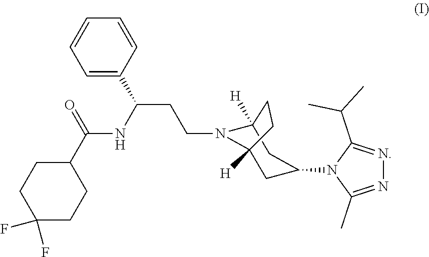 [SMILES: Cc1nnc(C(C)C)n1[C@@H]1C[C@H]2CC[C@@H](C1)N2CC[C@H](NC(=O)C1CCC(F)(F)CC1)c1ccccc1]

In an example embodiment, the medicament is formulated as a dosage form selected from the group consisting of tablets, capsules, and powders. In another example embodiment, the medicament is formulated to deliver the compound of Formula (II) in an extended-release or controlled-release dosage form. In an example, the medicament further comprises one or more pharmaceutically acceptable carriers or excipients.

The claimed methods and use of the invention advantageously provide a means for identifying senotherapeutic targets and for treating muscle degradation, for example muscle degradation associated with sarcopenia.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described, by way of example, with reference to the accompanying drawings in which:

FIGS. 1A and 1B are a schematics of methods of identifying senotherapeutic targets in accordance with one or more example embodiments.

FIGS. 2A through 2P illustrate multiomics mapping of a senescence atlas in aging human muscle, and are individually referenced below.

FIG. 2A is a uniform manifold approximation and projection (UMAP) plot showing 12 (sub)types of muscle resident mononucleated cell populations identified through analysing snRNA-seq.

FIG. 2B is an alluvial plot showing the distribution of young and aged cells across each of the above 8 main cell types. Pie plots below show the relative cell composition between old and young groups across each cell type.

FIG. 2C is an arc plot showing the degree of responsiveness of each cell type to ageing. The Augur score shows cell types based on their molecular response to ageing.

FIG. 2D is a boxplot illustrating transcriptional noise in young (Y) vs. aged (A) groups for the indicated cells arranged by decreasing order of A vs. Y ratio.

FIG. 2E shows scatter plots of muscle stem cells (MuSC), fibro-adipogenic progenitors (FAP), endothelial cells (EC), and macrophages (MP) proportions in each of the young and aged donors.

FIG. 2F shows a UMAP plot coloured by SenMayo ss-GSVA score (top); a UMAP plot showing senescent (Sn) vs. non-senescent (nSn) cells of MuSCs (middle); a bar plot showing the relative ratio of Sn vs. nSn cells between young and aged groups of MuSCs (bottom).

FIG. 2G shows a UMAP plot coloured by SenMayo ss-GSVA score (top); a UMAP plot showing senescent (Sn) vs. non-senescent (nSn) cells of FAP (middle); a bar plot showing the relative ratio of Sn vs. nSn cells between young and aged groups of FAP (bottom).

FIG. 2H shows a UMAP plot coloured by SenMayo ss-GSVA score (top); a UMAP plot showing senescent (Sn) vs. non-senescent (nSn) cells of EC (middle); a bar plot showing the relative ratio of Sn vs. nSn cells between young and aged groups of EC (bottom).

FIG. 2I shows a UMAP plot coloured by SenMayo ss-GSVA score (top); a UMAP plot showing senescent (Sn) vs. non-senescent (nSn) cells of smooth muscle cells (SMC) (middle); a bar plot showing the relative ratio of Sn vs. nSn cells between young and aged groups of SMC (bottom).

FIG. 2J is a dot plot showing representative GO terms of upregulated DEGs in Sn cells across the four cell types.

FIG. 2K is schematic of a method of senescence detection in human muscle or freshly isolated MuSCs from additional pairs of young and aged donors.

FIG. 2L shows H&E staining illustrating muscle atrophy in aged vs. young human muscle.

FIG. 2M is a IF staining of DAPI (blue), P16 (green) and P21 (red) performed on the collected human muscle sections.

FIG. 2N shows SA-β-GAL staining performed on MuSCs freshly isolated from young and aged muscles.

FIG. 2O is an IF staining of DAPI (blue), P16 (green) and PAX7 (red) performed on the above isolated and the percentage of P16+ cells is shown on the right.

FIG. 2P illustrates RT-qPCR detection of the mRNA expression of P14, P16, P19 and P21 genes in the above MuSCs.

FIG. 3A is a discriminative dimensionality reduction (DDR) tree visualization of MuSC trajectory with mapping of pseudotime.

FIG. 3B is a discriminative dimensionality reduction (DDR) tree visualization of MuSC trajectory with mapping of age group.

FIG. 3C is a discriminative dimensionality reduction (DDR) tree visualization of MuSC trajectory with mapping of senescence annotation.

FIG. 3D is a discriminative dimensionality reduction (DDR) tree visualization of MuSC trajectory with mapping of CDKN1A expression level information.

FIG. 3E is a plot showing the ss-GSVA score of cell cycle activity signature along MuSC ageing trajectory pseudotime. The solid yellow line is the local regression result for individual pseudotime bins (55 total, sized 0.10 per bin), with the grey shadow depicting the 95% confidence intervals (CIs).

FIG. 3F is a heat map visualisation of expression levels of genes (right) with correlated expression profiles to MuSC ageing pseudotime ordered from Early to Late stage.

FIG. 3G is a heat map visualisation of expression levels of DEGs between Late 1 and Late 2 fates ordered by pseudotime.

FIG. 3H is a DDR tree visualization of FAP trajectory with mapping of pseudotime.

FIG. 3I is a DDR tree visualization of FAP trajectory with mapping of senescence annotation.

FIG. 3J is a plot showing the ss-GSVA score of cell cycle activity signature along FAP aging trajectory pseudotime.

FIG. 3K is a heat map visualization of expression levels of genes with correlated expression profiles to FAP ageing pseudotime from early to late stage.

FIG. 3L is a DDR tree visualization of EC trajectory with mapping of pseudotime.

FIG. 3M is a DDR tree visualization of EC trajectory with mapping of senescence annotation.

FIG. 3N is a plot showing the ss-GSVA score of cell cycle activity signature along EC ageing trajectory.

FIG. 3O is a heat map visualization of expression levels of genes with correlated expression profiles to EC aging pseudotime.

FIG. 3P is a DDR tree visualization of SMC trajectory with mapping of pseudotime.

FIG. 3Q is a DDR tree visualization of SMC trajectory with mapping of senescence annotation.

FIG. 3R is a plot showing the ss-GSVA score of cell cycle activity signature along SMC ageing.

FIG. 3S is a heat map visualization of expression levels of genes with correlated expression profiles to SMC ageing pseudotime.

FIG. 4A shows circos plots showing up-regulated and down-regulated DEGs in senescent MuSCs, FAPs, ECs, or SMCs (total numbers indicated in the brackets). Each connecting curve represents an up- or down-regulated Sn-DEG shared by two cell types; light and dark grey arc (inner circle) indicates cell type-unique and shared Sn-DEG, respectively.

FIG. 4B is an upset plot showing the numbers of cell type-unique and shared up-regulated SASP genes for pairwise comparisons among the indicated cell types.

FIG. 4C is a plot showing the up-regulated SASPs shared by at least two cell types.

FIG. 4D is a scatter plot showing the up-regulated SASPs specific in each cell type.

FIG. 4E is a ridge map showing the distribution density of ss-GSVA score for classical SASP genes in the senescent (Sn) vs. non-senescent (nSn) cells.

FIG. 4F is a ridge map showing the distribution density of ss-GSVA score for classical SASP genes in aged vs. young cells.

FIG. 4G is a heat map of top-ranked up-regulated SASPs in Sn vs. nSn cells.

FIG. 4H is a heat map of top-ranked up-regulated SASPs in aged vs. young cells.

FIG. 4I illustrates RT-qPCR detection of the expression level of top-ranked up-regulated SASP genes between aged and young human MuSCs.

FIG. 4J is a bar plot comparing the interaction strength of SASP-mediated intercellular communications in aged vs. young group.

FIG. 4K is a heat map showing differential number of SASP-mediated interactions between two cell types. Red/Blue represents increased/decreased signalling in the aged vs. young.

FIG. 4L shows relative flows of differentially active signalling pathways during muscle ageing, annotated with their respective functions.

FIG. 4M is a plot showing the signal strength change by aggregating all L-R pairs within CXCL pathway. The edge colour corresponds to the sender cell type, and the edge weight is proportional to the interaction strength.

FIG. 4N is a chord diagram visualising MuSC-centred cell-cell communication for a set of up-regulated SASP ligands/receptors, with MusCs designated as the receptor cell type.

FIG. 4O is a chord diagram visualising MuSC-centred cell-cell communication for a set of up-regulated SASP ligands/receptors, with MuSCs designated as the sender cell type.

FIG. 4P is a dot plot showing the increased SASP L-R pairs from MuSC (sender cell) to other cells (target cell) in aged vs. young group.

FIG. 5A is a snRNA-seq analysis of the expression of CCL3, CCL4, CCL5 and CCR5 (CCR5 axis genes) in human MuSCs.

FIG. 5B is a RT-qPCR detection of the expression level of CCR5 axis genes in MuSCs freshly isolated from additional pairs of young and aged donors.

FIG. 5C is a schematic of high dose short term (HDST) treatment/assessment regime of DMSO or MVC in ageing mice.

FIG. 5D is a bar chart showing ratio of TA/body weight of the above-treated mice.

FIG. 5E shows H&E staining of tibialis anterior (TA) muscles collected from the above-treated mice. The bar chart on the right shows the quantification of CSAs of the stained fibers.

FIG. 5F shows treated mice subjected to a grip strength meter with final strength shown.

FIG. 5G shows treated mice subjected to a grip strength meter with strength changes shown.

FIG. 5H shows treated mice subjected to treadmill exercise and the maximal running speed recorded.

FIG. 5I shows treated mice subjected to treadmill exercise and the maximal distance recorded.

FIG. 5J is a flow cytometry detection of MuSC, MP, and FAP populations in treated mice.

FIG. 5K shows scRNA-seq performed on the mononucleated cells isolated from three pairs of DMSO/MVC-treated mice. Unsupervised clustering resolved at least 13 cell types (color-coded).

FIG. 5L shows sankey plots (top) showing the distribution of DMSO and MVC cells across each cell type. Pie plots showing the relative proportion of each cell type between DMSO and MVC groups is illustrated at the bottom

FIG. 5M is a bar plot (top) showing the USS-defined relative percentage of senescent vs. non-senescent MuSCs in DMSO vs. MVC. A violin plot showing the relative expression level of p21 in the cells is shown at the bottom.

FIG. 5N is a bar plot (top) showing the USS-defined relative percentage of senescent vs. non-senescent FAPs in DMSO vs. MVC. A violin plot showing the relative expression level of p21 in the cells is shown at the bottom.

FIG. 5O is a bar plot (top) showing the USS-defined relative percentage of senescent vs. non-senescent ECs in DMSO vs. MVC. A violin plot showing the relative expression level of p21 in the cells is shown at the bottom.

FIG. 5P is a bar plot (top) showing the USS-defined relative percentage of senescent vs. non-senescent SMC in DMSO vs. MVC. A violin plot showing the relative expression level of p21 in the cells is shown at the bottom.

FIG. 5Q is a bar plot showing SASP-mediated interaction strength of DMSO vs. MVC calculated by CellChat.

FIG. 5R is a heat map showing the SASP-mediated cell-cell interaction pairs between two cell types altered between MVC vs. DMSO treatment. The red/blue colour indicates the increased or decreased interaction, respectively.

FIG. 5S shows the interaction frequency of Ccl3-Ccr5 and Ccl4-Ccr5 pairs analysed in MVC vs. DMSO group.

FIG. 5T shows RT-qPCR detection of the expression levels of Ccl3, Ccl4, Ccl5 and Ccr5 in whole muscle in MVC vs. DMSO group.

FIG. 5U shows a bulk RNA-seq performed in freshly isolated MuSCs from DMSO or MVC-treated mice and up- or down regulated DEGs were identified using Log2FC>0.5 as a cut off.

FIG. 5V is a GO analysis of the identified 231 down-regulated DEGs.

FIG. 5W is a GSEA analysis of the repressed SASP expression in the MVC-treated MuSCs.

FIG. 5X shows RT-qPCR detection of the expression levels of Ccl3, Ccl4, Ccl5 and Ccr5 in the above MuSCs.

FIG. 6A is a plot showing the predicted transcription factors (TFs) governing senescence state shared by at least two cell types.

FIG. 6B is a box plot showing predicted ATF3 ATAC accessibility level in senescent vs. non-senescent cells.

FIG. 6C is a box plot showing predicted ATF3 ATAC accessibility level in aged vs. young groups.

FIG. 6D is a box plot showing the ss-GSVA gene set scores of target genes activated by ATF3.

FIG. 6E is a box plot showing the ss-GSVA gene set scores of target genes repressed by ATF3.

FIG. 6F is a network visualisation of representative GO terms and pathways of ATF3-modulated DEGs in each cell type of aged vs. young muscle. The nodes represent GO terms or pathways, and the pie plots display the proportion of genes corresponding to a specific GO term or pathway in each cell type.

FIG. 6G is a network visualisation of ATF3 targeted up- or down-regulated senescent genes in each cell type. Node size positively correlates with the number of cell types with its embedded pie chart indicating the number of up- and down-regulated DEGs. Each connecting line represents Sn-DEGs in the corresponding cell type with its colour indicating log2fold change (FC) values.

FIG. 6H is a network visualisation of core activator TFs in each cell type between old and young groups. Outer nodes display different cell types, and the node colour represents regulation score of all TF-SASP associations averaged on each cell type. Inner nodes positively correlate with the number of all TF-SASP pairs for each cell type. Each connecting line represents the number of SASP factors regulated by certain TF for each cell type.

FIG. 6I is a network visualisation of repressor TFs in each cell type between old and young groups. Outer nodes display different cell types, and the node colour represents regulation score of all TF-SASP associations averaged on each cell type. Inner nodes positively correlate with the number of all TF-SASP pairs for each cell type. Each connecting line represents the number of SASP factors regulated by certain TF for each cell type.

FIG. 6J is a heat map showing JUNB-SASP regulation score for each cell type.

FIG. 6K shows heat maps highlighting smoothed normalized JUNB DORC accessibility, SCTnormalized RNA expression, and DORC-RNA difference for JUNB target SASP genes in MuSCs.

FIG. 7A is a heat map showing snATAC-seq detected DORC regulation scores for top-ranked TF-SASP association in human MuSCs.

FIG. 7B is a violin plot showing the snATAC-seq detected chromatin accessibility level of predicted human (h) JUNB binding sites in young vs. aged human MuSCs.

FIG. 7C is a violin plot showing the normalized expression level of hJUNB in young vs. aged human MuSCs.

FIG. 7D shows MuSCs freshly isolated from young and aged donors for RT-qPCR detection of the expression levels of hJUNB and SASP targets.

FIG. 7E shows MuSCs freshly isolated from young (2 m) and aged (24 m) mice for RTqPCR detection of the expression levels of mouse (m)JunB and SASP targets.

FIG. 7F shows MuSCs freshly isolated from Ctrl or JunB-iKO mice for RT-qPCR detection of the expression levels of mJunB SASP targets.

FIG. 7G shows MuSCs isolated from young mice and transfected with Ctrl or JunB overexpressing plasmid and RT-qPCR detection of the expression levels of mJunB SASP targets in the above cells.

FIG. 7H are pie charts showing the distribution of snATAC-seq predicted hJUNB binding sites in young and aged human MuSCs.

FIG. 7I show MuSCs freshly isolated from young (2 m) and aged mice (24 m) and JunB CUT&RUN-seq was performed; pie charts show the distribution of mJunB binding.

FIG. 7J is an average profile plot showing the H3K27ac signal surrounding the above detected mJunB binding sites (+/−1000 bp).

FIG. 7K is a pie chart showing the overlapping of mJunB potential target genes (promoter and enhancer bound) in young and aged mice MuSCs.

FIG. 7L is a pie chart showing the overlapping of the mJunB target genes and classical SASP genes. The young and aged unique SASP targets are listed.

FIG. 7M show genomic snapshots on Cxcl1 locus showing the binding peaks of mJunB and H3K27ac in young and aged MuSCs.

FIG. 7N is a genomic coverage plot on CXCL1 locus showing the predicted hJUNB binding peaks in Sn vs. nSn human MuSCs.

FIG. 7O is a genomic coverage plot on CXCL1 locus showing the predicted hJUNB binding peaks in young vs. aged human MuSCs.

FIG. 7P illustrates chromatin openness level (DORC) versus normalised gene expression (SCT) dynamics of CXCL1 gene along MuSC aging pseudotime. Dotted line represents LOESS fit to the values obtained from sliding window bin averaged from DORC accessibility or SCT expression levels (n=100 cells per bin).

FIG. 8A is a schematic of the experimental design for inducing senescence in IMR-90 human fibroblast by treating cells with Etoposide (ETO) or passaging for 10 (P10) generations (top). Bottom images shows SA-β-GAL staining showing the senescent IMR-90 cells ETO treatment or P10. Bar chart on the right shows the quantification of the SA-β-GAL+ cells.

FIG. 8B is a bar chart showing RT-qPCR detection of the expression levels of senescent marker genes (P14, P16, P19, P21) in the above-treated cells.

FIG. 8C is a bar chart showing RT-qPCR detection of the expression levels of JUNB target genes in the above-treated cells.

FIG. 8D shows a schematic of the experimental design for testing the effect of JUNB knockdown by siRNA oligos on ETO-induced senescent IMR-90 cells (shown on the left). The right shows RTqPCR confirmation of the JUNB knockdown in the above cells.

FIG. 8E shows SA-β-GAL staining showing the percentage of senescent cells after the JUNB knockdown.

FIG. 8F is a bar chart showing RT-qPCR detection of the expression levels of senescent marker genes in the above cells.

FIG. 8G is a bar chart showing RT-qPCR detection of the expression level of CXCL1 in the above cells.

FIG. 8H is a schematic of the experimental design for testing the effect of JUNB knockdown by siRNA oligos in P10 senescent IMR-90 (left). On the right is RT-qPCR confirmation of the expression level of JUNB in the above-treated cells.

FIG. 8I illustrates SA-β-GAL staining showing the percentage of senescent cells after the JUNB knockdown.

FIG. 8J is bar chart showing RT-qPCR detection of the expression levels of senescent marker genes in the above cells.

FIG. 8K is bar chart showing RT-qPCR detection of the expression level of CXCL1 in the above cells.

FIG. 8L is a schematic of the experimental design for testing the effect of JUNB inhibition by T5224 treatment in P10 senescent IMR-90 cells (shown on the left). A bar chart on the right shows RT-qPCR detection of the expression level JUNB in the above-treated cells.

FIG. 8M illustrates SA-β-GAL staining showing the percentage of senescent cells in the above-treated cells.

FIG. 8N is a bar chart showing RT-qPCR detection of the expression levels of senescent marker genes in the above cells.

FIG. 8O is a bar chart showing RT-qPCR detection of the expression level of CXCL1 in the above cells.

FIG. 8P shows a schematic of the experimental design for testing the effect of JUNB inhibition by T5224 treatment in P10 senescent IMR-90 cells (shown on the left). On the right is a bar chart showing RT-qPCR detection of the expression level of JUNB in the above cells.

FIG. 8Q illustrates SA-β-GAL staining showing the percentage of senescent cells in the above cells.

FIG. 8R is a bar chart showing RT-qPCR detection of the expression levels of senescent marker genes in the above cells.

FIG. 8S is a bar chart showing RT-qPCR detection of the expression level of CXCL1 in the above cells.

FIG. 8T is a schematic of the experimental design for conducting JUNB CUT&RUN-seq in Ctrl and ETO-treated senescent IMR-90.

FIG. 8U shows pie charts showing the distribution of the identified JUNB binding sites in the above cells.

FIG. 8V is a pie chart showing the overlapping of the JUNB binding with H3K27Ac in the above cells.

FIG. 8W is a pie chart showing the overlapping of JUNB target genes in Ctrl and ETO cells.

FIG. 8X is a pie chart showing the overlapping of the identified JUNB SASP targets in Ctrl and ETO cells.

FIG. 8Y is a pie chart showing the overlapping of JUNB SASP targets regulated via E-P looping in Ctrl and ETO cells.

FIG. 8Z is genomic snapshots on CXCL1 locus showing JUNB mediated E-P looping in Ctrl and ETO cells. A new E-P loop was gained in ETO cells.

FIG. 9 is a schematic of a treatment regime whereby 10 mg/kg of the composition comprising a therapeutically effective amount of a compound of Formula (II) (indicated as T5224) is administered every alternate day to 17-month old mice intraperitoneally for 1 month and harvested.

FIG. 10A is a bar chart comparing body weight following treatment with DMSO and the composition comprising a therapeutically effective amount of the compound of Formula (II).

FIG. 10B is a bar chart comparing total anatomical weight (TA weight) following treatment with DMSO and the composition comprising a therapeutically effective amount of the compound of Formula (II).

FIG. 10C is a bar chart comparing total anatomical weight (TA weight)/body weight following treatment with DMSO and the composition comprising a therapeutically effective amount of the compound of Formula (II).

FIG. 11A is SA-β-GAL staining illustrating T5224 treated mice muscle show healthy muscle morphology and increased muscle fibre size.

FIG. 11B is a bar chart showing muscle cross-sectional area following treatment with DMSO and T5224 illustrating T5224 treated mice muscle show healthy muscle morphology and increased muscle fibre size.

FIG. 12A is a schematic illustrating experimental design for testing grip strength in mice.

FIG. 12B is a bar chart comparing grip strength in mice following treatment with DMSO and T5224 illustrating that muscle function was significantly enhanced following treatment with T5224 as evidenced by the notable increase in grip strength.

FIG. 13A is a schematic illustrating experimental design for a treadmill running test in mice.

FIG. 13B is a bar chart comparing running speed following treatment with DMSO and T5224 in mice injected with a composition comprising a therapeutically effective amount of the compound of Formula (I) (MVC).

FIG. 13C is a bar chart showing running distance following treatment with DMSO and T5224 in mice injected with a composition comprising a therapeutically effective amount of the compound of Formula (I) (MVC).

### DETAILED DESCRIPTION

Population ageing is a growing global concern, particularly in relation to healthcare for the elderly. To illustrate the scale of this global issue, using Hong Kong as a case study, in 2024 the group aged 60 years and over accounts for 31.4% of the total Hong Kong population and this uptrend is only likely to rise.

One of the salient aspects of ageing is the decline in skeletal muscle tissue, a condition known as sarcopenia. This process involves both the loss of muscle mass and function, which significantly affects the quality of life in older adults. Sarcopenia is primarily driven by cellular senescence which relates to the irreversible cell cycle arrest that occurs in ageing cells, particularly muscle cells. Sarcopenia is one of the most prevalent geriatric conditions globally, affecting approximately 14.4-16.1% of individuals aged 60 and older. This condition imposes significant socioeconomic and healthcare burdens due to its various clinical and societal consequences, including:
Deterioration in quality of life and loss of independence
Elevated risk of falls, fractures, and increased mortality
Greater susceptibility to frailty and associated healthcare costs

Concerningly, there are currently no effective small molecule drugs targeting ageing-related sarcopenia and small molecule drug targets for treating this condition are urgently desired. The potential number of target users of such a drug are estimated to be at least 0.4 million in Hong Kong alone, and over 60 million people in Mainland China.

The inventors have advantageously studied and understood the mechanism of cellular senescence in ageing muscle to develop interventions to prevent or mitigate sarcopenia.

The inventors have found that cellular senescence is a key hallmark and driver of tissue and organism ageing. Cellular senescence, in muscle fibres, leads to an accumulation of dysfunctional cells that do not contribute to muscle regeneration or repair, which accelerates muscle wasting. The inventors have found that intervention for this condition may be in the form of senotherapeutics—for example, senolytics that selectively kill senescent cells, or senomorphics that suppress senescence-associated secretory phenotypes (SASPs) which both act as a means to alleviate ageing-related disease and enable healthy ageing.

The inventors have resourcefully addressed this issue using multimodal mapping. They have advantageously developed a senescence atlas for the identification of novel senotherapeutic targets and a method of treating muscle degradation in a subject. The research and development involved in this project has involved a number of crucial steps, including target identification, compound screening, and lead identification. This has been followed by preclinical studies, including in vitro studies, in vivo studies, and toxicity testing. Validation testing will also require Phase I, II, and III clinical trials and dosage and safety monitoring before review and approval of this treatment.

An integrated single-nucleus RNA sequencing and ATAC sequencing approach was developed to define the senescence atlas in ageing human muscle. The inventors, in their study, uncovered the heterogeneity, temporal dynamics, functions, and regulatory mechanisms of senescence in ageing muscle. Additionally, potential senotherapeutic targets were identified, including the CCLs/CCR5 axis and AP-1 proteins. Advantageously, the inventors repurposed the compound of Formula (I) (maraviroc) as a pharmacological senotherapeutic for sarcopenia and rejuvenation.

Further, the inventors found in their study that scATAC-seq identified AP-1 family transcription factor (TF) as important senescence regulators in in ageing muscle. They beneficially discovered that a compound of Formula (II) targeting AP-1 can be used as a senotherapeutic for the treatment of sarcopenia. Further details are discussed below.

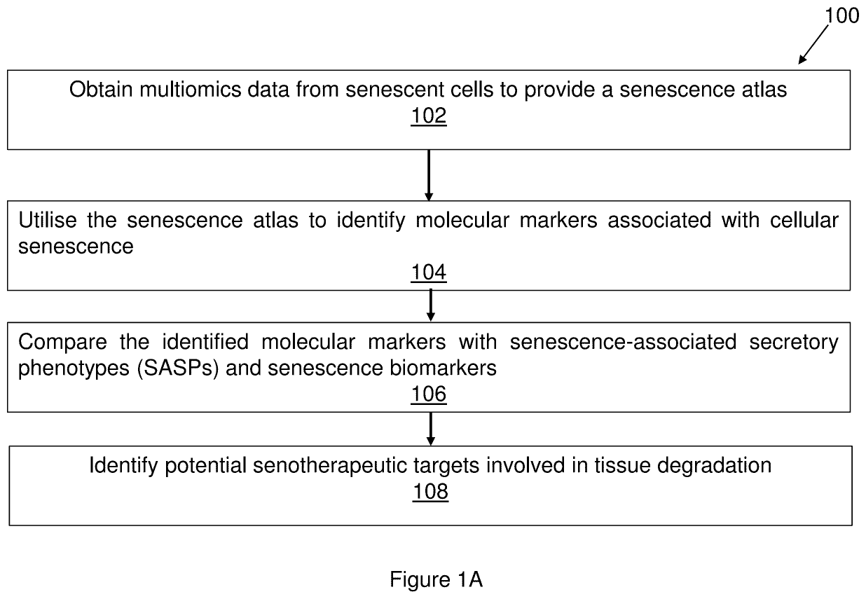

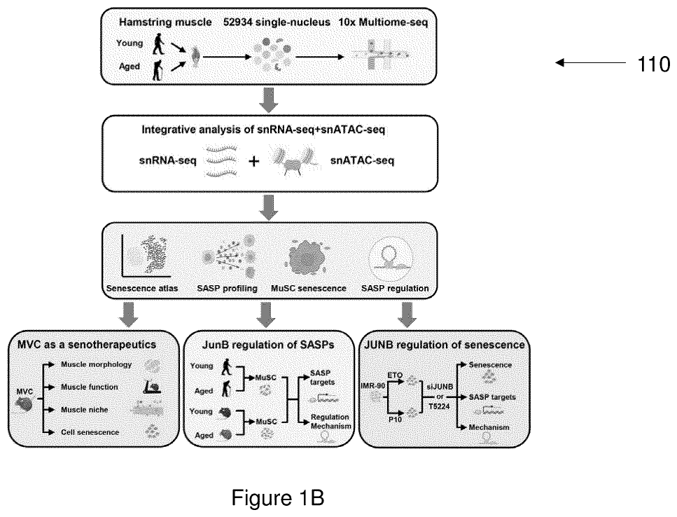

With reference to FIG. 1A and FIG. 1B 110, an embodiment of the present invention is illustrated. This embodiment is arranged to provide a method 100 for identifying senotherapeutic targets, comprising the steps of: obtaining multiomics data from senescent cells to provide a senescence atlas 102, utilising the senescence atlas to identify molecular markers associated with cellular senescence 104, comparing the identified molecular markers with senescence-associated secretory phenotypes (SASPs) and senescence biomarkers 106, and identifying potential senotherapeutic targets involved in tissue degradation 108. The inventors have advantageously created a detailed atlas of senescence in muscle cells, identifying common and distinct SASPs and examining how these SASPs affect cellular interactions and tissue function. For example, SASPs include a diverse range of factors such as pro-inflammatory cytokines like interleukins, tumour necrosis Factoria-alpha, and Interferon-gamma. SASPs also include growth factors, like fibroblast growth factor and vascular endothelial growth factor, and matrix metalloproteinases, and chemokines like CCL2, CCL5, and CXCL1.

In an example embodiment, the molecular markers include one or more genes, proteins, epigenetic modifications, or secretory factors associated with senescence.

In one example, the multiomics data includes one or more transcriptomic, epigenomic, or proteomic profiles. Multiomics data provides a comprehensive and detailed understanding of cellular and tissue states. Transcriptomic profiles provide insights into gene expression levels, epigenomic data provides insight into how gene expression is regulated by examining chemical modifications to DNA and histone proteins, and proteomic data provides insights into the functional molecules that carry out cellular processes.

In an example embodiment, the multiomics data is obtained using single-nucleus sequencing or other single-cell omics technologies.

Preferably, the method 100 further includes a step of validating the identified potential senotherapeutic targets 108 by assessing the role in cellular senescence in vitro or in vivo. Preferably, the method 100 further includes a step of determining the effects of targeting the potential senotherapeutic targets 108 on cellular senescence. The potential targets include, for example, genes, proteins, or signalling pathways involved in one or more of cellular senescence, inflammation, muscular degradation, sarcopenia, or age-related diseases.

For example, the senescence atlas 102 is used to compare senescent and non-senescent cell profiles to identify specific alterations associated with senescence.

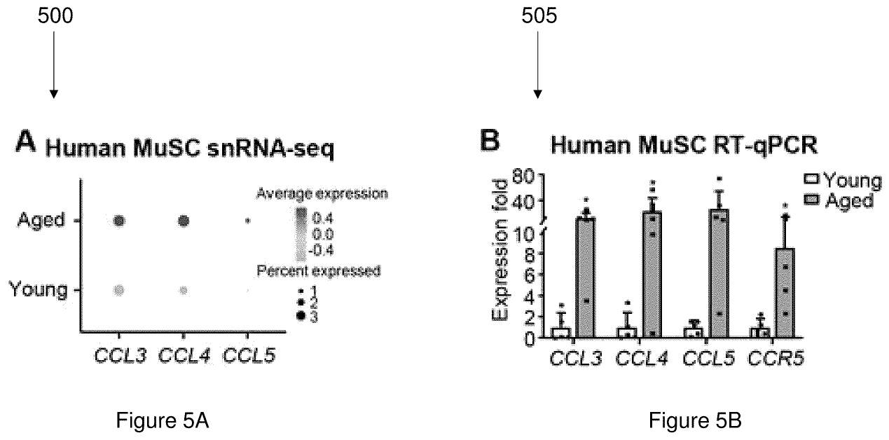

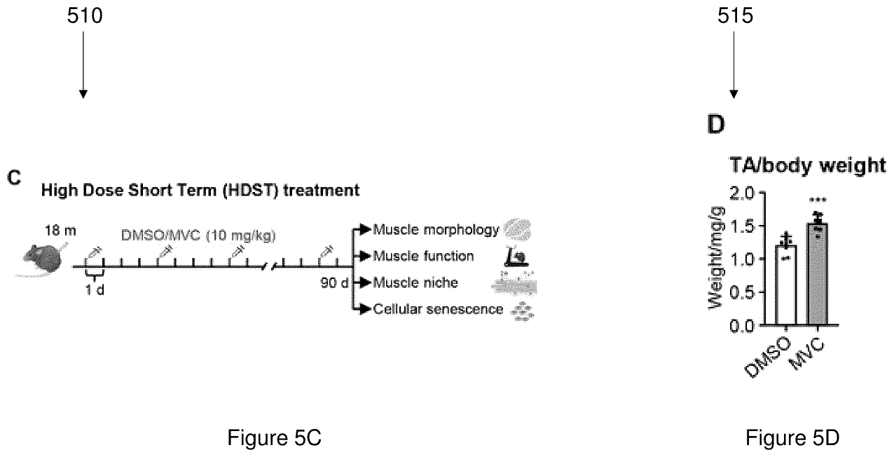

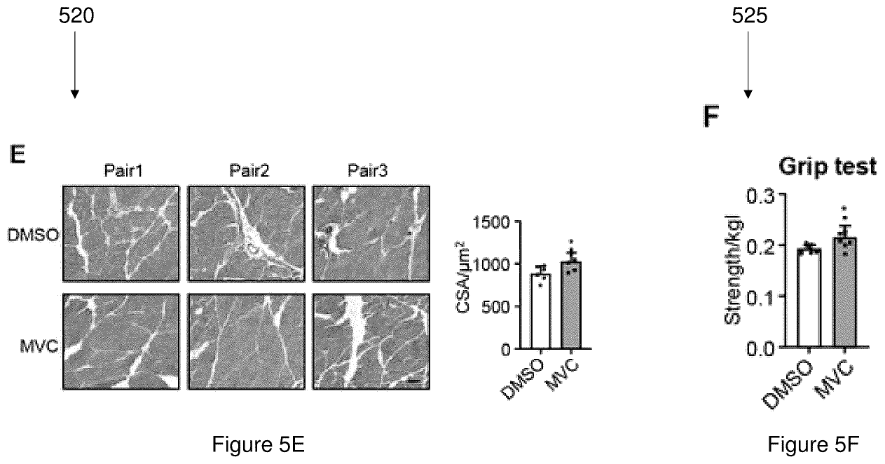

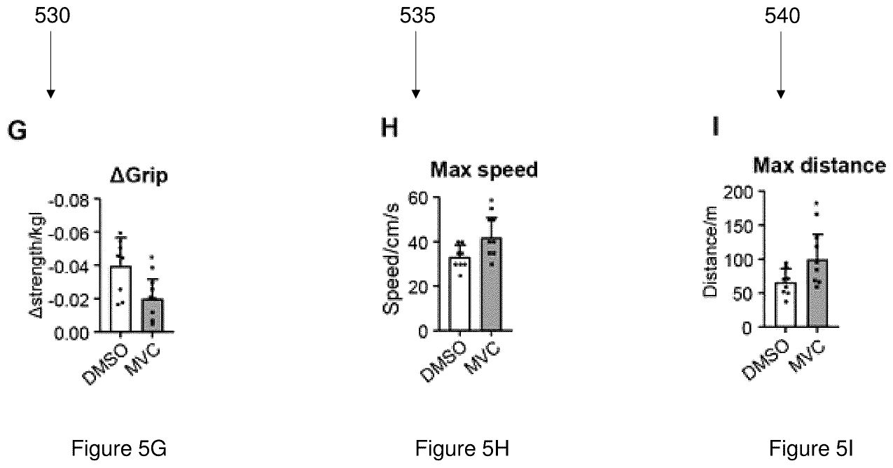

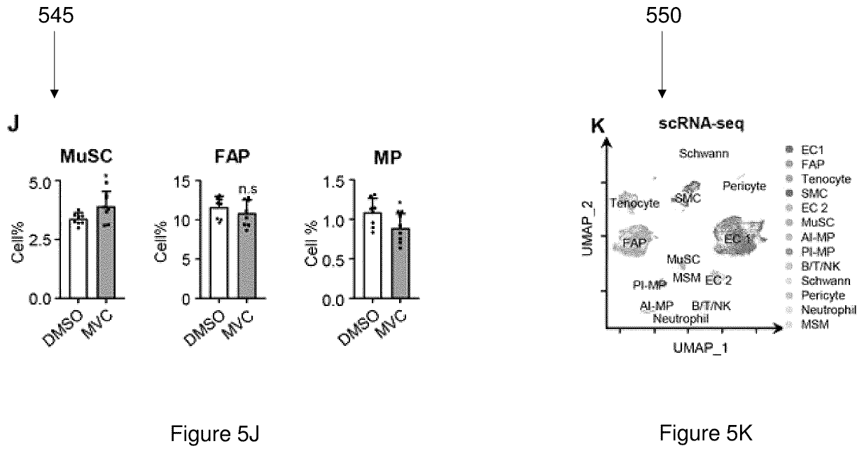

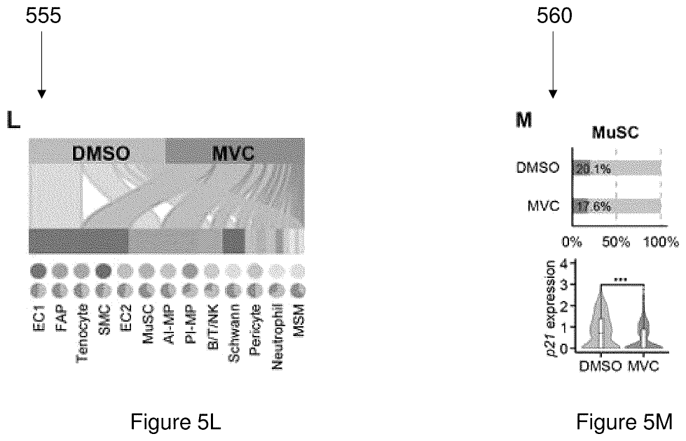

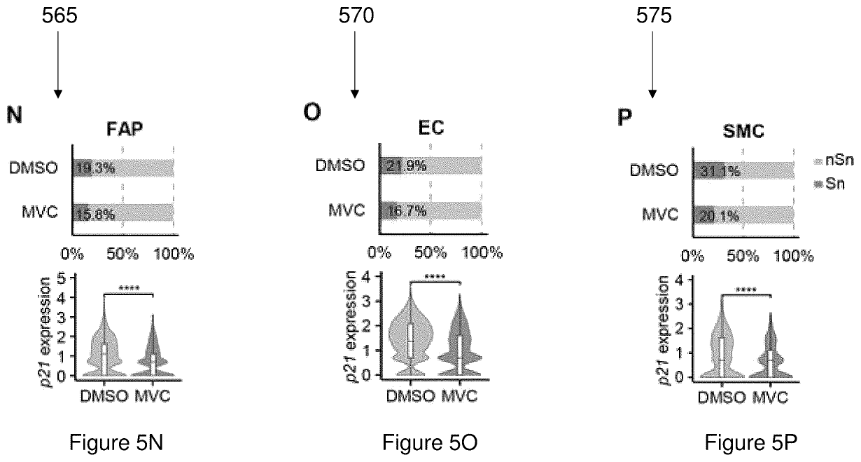

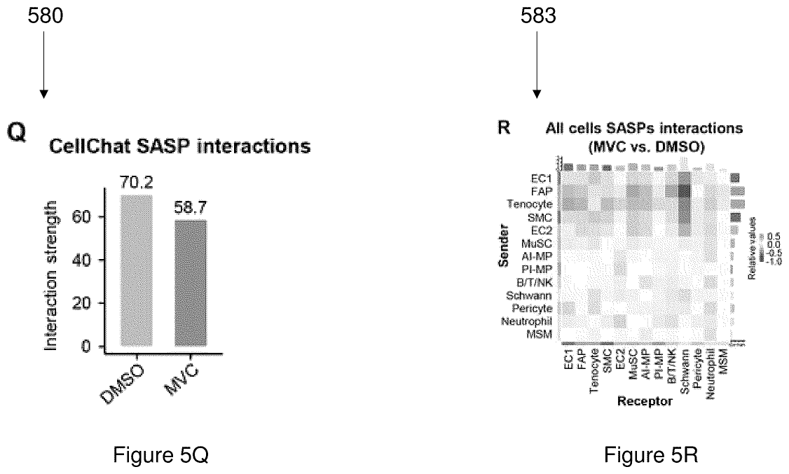

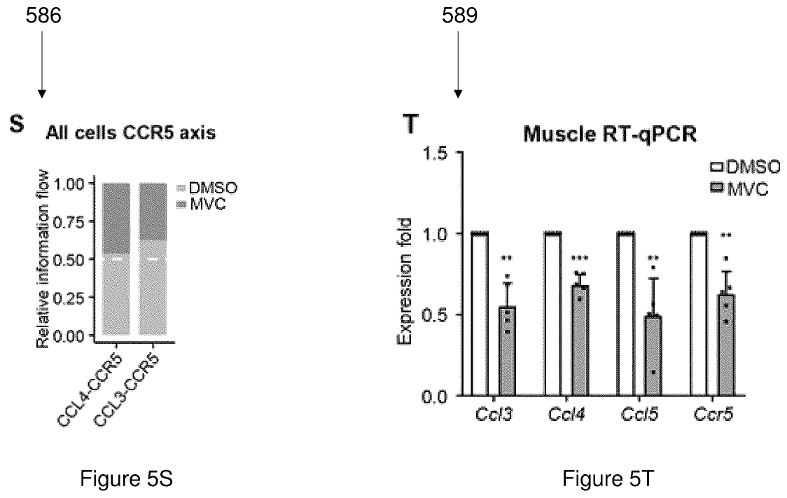

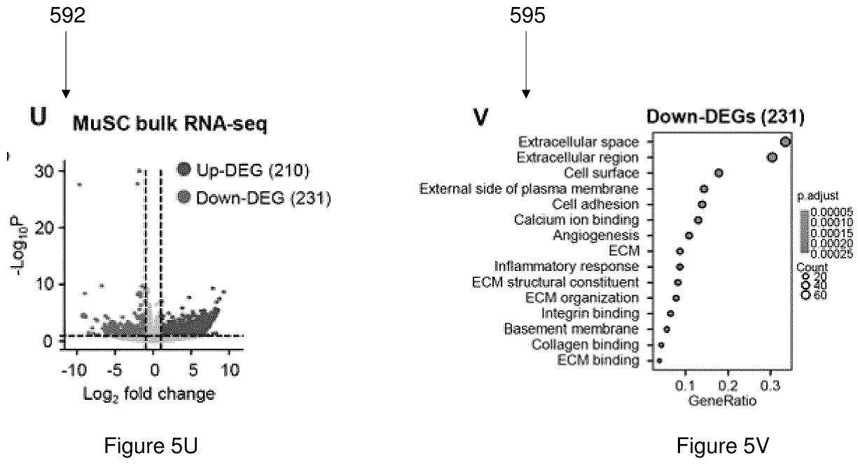

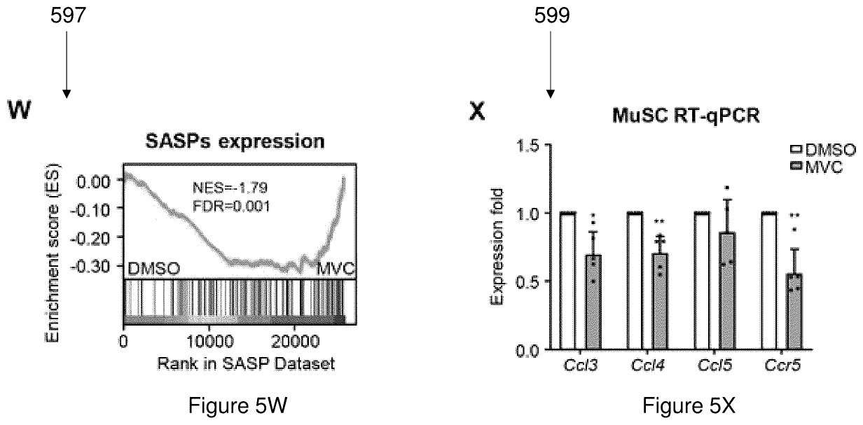

With respect to FIG. 5A 500-5X 599 there is provided a method of treating muscle degradation in a subject, the method comprising administering to the subject a composition comprising a therapeutically effective amount of a compound of Formula (I), or a pharmaceutically acceptable salt, solvate, or hydrate thereof:

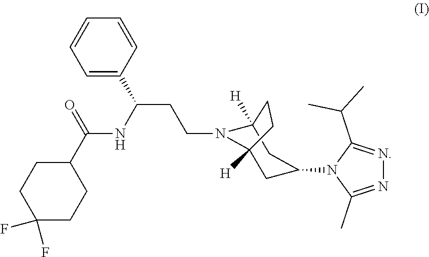 [SMILES: Cc1nnc(C(C)C)n1[C@@H]1C[C@H]2CC[C@@H](C1)N2CC[C@H](NC(=O)C1CCC(F)(F)CC1)c1ccccc1]

In a preferred embodiment, the composition includes one or more pharmaceutically acceptable carriers or excipients.

The composition reduces or delays cellular senescence in the subject. The inventors found that treatment with the composition of Formula (I) led to an increase in muscle mass and morphology as shown in FIGS. 5C 510, 5D 515, and 5E 520.

In an example embodiment, the composition targets cellular senescence mechanisms to mitigate muscle degradation and improve muscle function in the subject. For example, the composition targets muscle atrophy or other related mechanisms to improve muscle function and reduce functional decline.

In one example, the composition targets SASPs to mitigate muscle degradation and improve muscle function in the subject. SASPs have an effect on inflammation by recruiting immune cells and promoting a pro-inflammatory environment, and tissue remodelling by altering the extracellular matrix, impacting tissue structure and function.

For example, the composition targets one or more senotherapeutic targets including genes, proteins, or signalling pathways involved in one or more of cellular senescence, inflammation, muscular degradation, sarcopenia, neurodegenerative diseases, cardiovascular ageing, or age-related diseases. The composition targets senescence biomarkers to mitigate muscle degradation and improve muscle function in the subject.

The therapeutically effective amount of the compound of Formula (I) administered is determined based on an assessment of degree of the muscle degradation in the subject and a severity of cellular senescence in the subject. Further, the composition is formulated for administration in the subject via oral, injectable, or topical routes. The composition may be administered in combination with one or more therapeutic agents or supplementary treatments, known as additional treatment, targeting muscle regeneration or senescence. The additional treatment may improve the efficacy of the treatment. The therapeutic agents include, for example, muscle growth stimulants, anti-inflammatory agents, or other senotherapeutic compounds, or a combination thereof.

The muscle degradation the subject of the treatment, is for example, associated with sarcopenia, muscle wasting disorders, age-related muscular weakness, or a combination thereof. Most preferably, the muscle degradation is associated with sarcopenia.

In one embodiment, the subject is an elderly individual or subject diagnosed with muscle loss, for example age-related muscle loss.

A further aspect of the invention provides the use of a compound of Formula (I), or a pharmaceutically acceptable salt, solvate, or hydrate thereof, in the manufacture of a medicament for the treatment of muscle degradation in a subject:

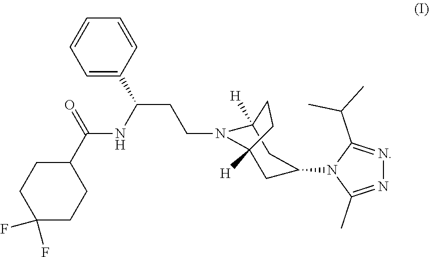 [SMILES: Cc1nnc(C(C)C)n1[C@@H]1C[C@H]2CC[C@@H](C1)N2CC[C@H](NC(=O)C1CCC(F)(F)CC1)c1ccccc1]

In one embodiment, a therapeutically effective amount of the medicament is administered to mitigate muscle degradation and improve muscle function.

In a preferred embodiment, the medicament reduces or delays cellular senescence. For example, the medicament is formulated to modulate senescence-associated secretory phenotypes (SASPs) in muscle cells.

The medicament targets one or more senotherapeutic targets including genes, proteins, or signalling pathways involved in one or more of cellular senescence, inflammation, muscular degradation, sarcopenia, neurodegenerative diseases, cardiovascular ageing, or age-related diseases. For example, the muscle degradation is associated with sarcopenia, muscle wasting disorders, age-related muscular weakness, or a combination thereof. Preferably, the muscle degradation is associated with sarcopenia.

In an example embodiment, the medicament is formulated as a dosage form selected from the group consisting of tablets, capsules, and powders. The medicament is formulated to deliver the compound of formula (I) in an extended-release or controlled-release dosage form. Preferably, the medicament further comprises one or more pharmaceutically acceptable carriers or excipients.

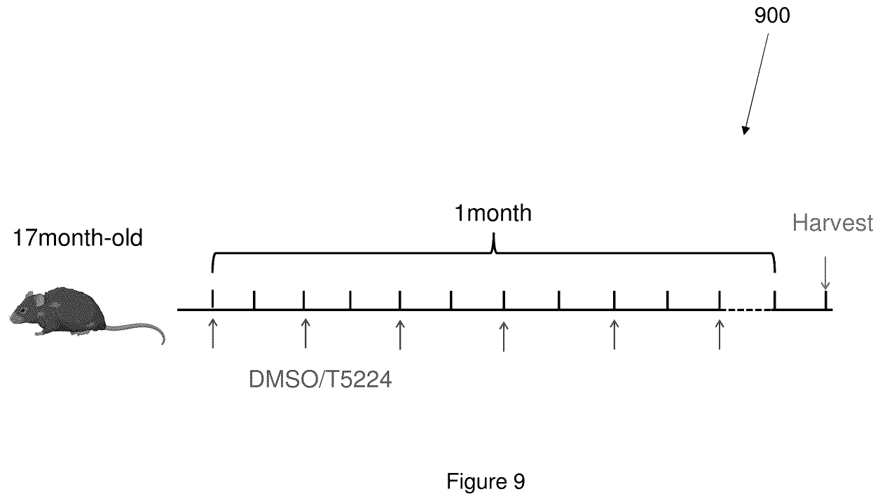

With reference to FIGS. 9-13, there is also provided a method of treating muscle degradation in a subject, the method comprising administering to the subject a composition comprising a therapeutically effective amount of a compound of Formula (II), or a pharmaceutically acceptable salt, solvate, or hydrate thereof:

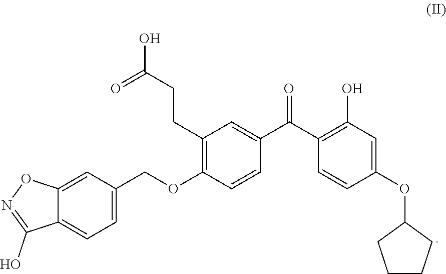 [SMILES: O=C(O)CCc1cc(C(=O)c2ccc(OC3CCCC3)cc2O)ccc1OCc1ccc2c(O)noc2c1]

For example, the muscle degradation is associated with sarcopenia, muscle wasting disorders, age-related muscular weakness, or a combination thereof.

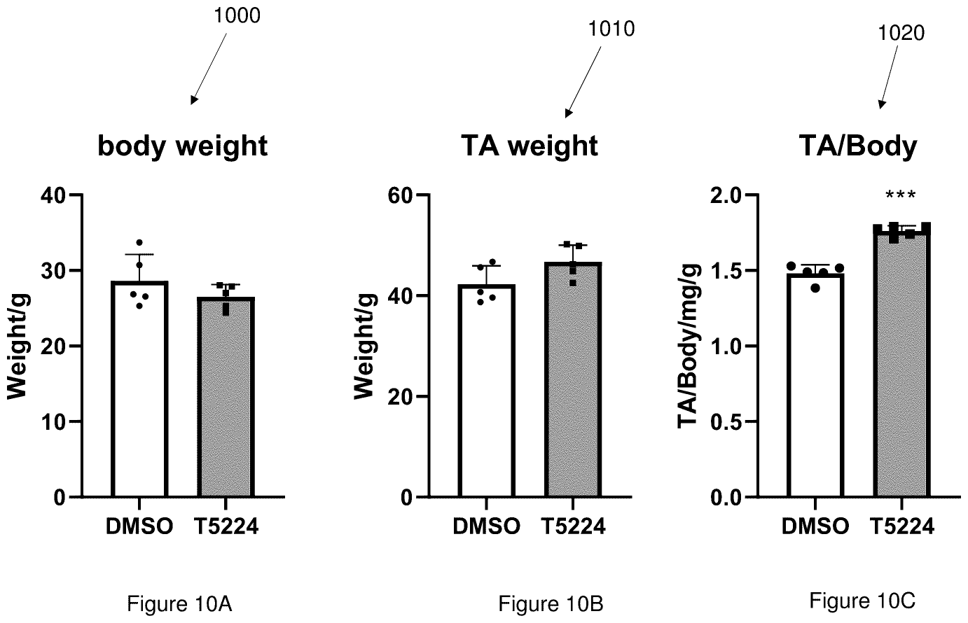

FIG. 9 900 illustrates a schematic 900 of the treatment regime 900 with the composition comprising a therapeutically effective amount of a compound of Formula (II). In an example, 10 mg/kg of the composition comprising a therapeutically effective amount of a compound of Formula (II) was administered every alternate day on 17-month-old mice intraperitoneally for 1 month and harvested for analysis. The composition comprising a therapeutically effective amount of a compound of Formula (II) advantageously targets cellular senescence mechanisms to mitigate muscle degradation and improve muscle function in the subject. With reference to FIGS. 10A 1000, 10B 1010, and 10C 1020, there is no significant change in the body weight (FIG. 10A 1000) between DMSO and composition comprising Formula (II) (T5224). However, treatment with the composition comprising a therapeutically effective amount of a compound of Formula (II) did result in an elevated total anatomical (TA) body weight (FIG. 10B 1010) and TA/body (FIG. 10C 1020) demonstration the administration of the composition comprising a therapeutically effective amount of a compound of Formula (II) leads to an increased muscle mass.

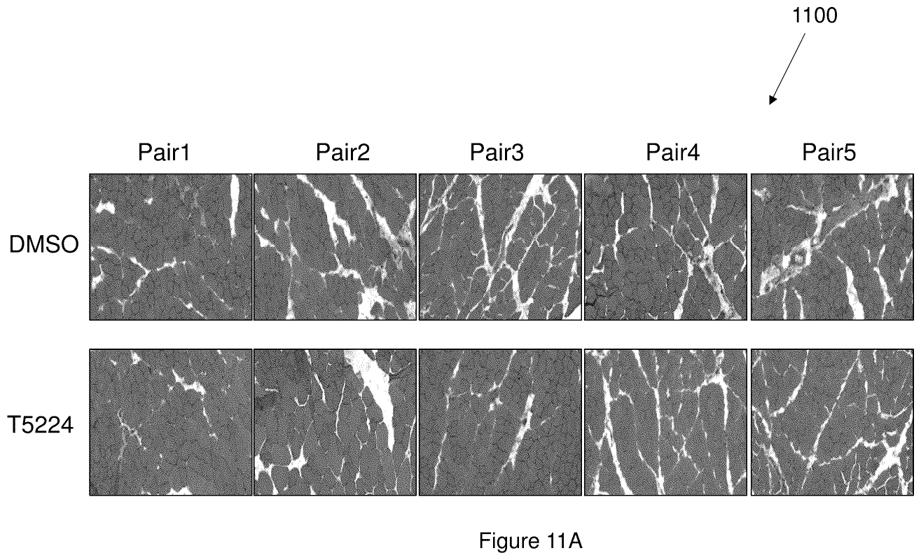

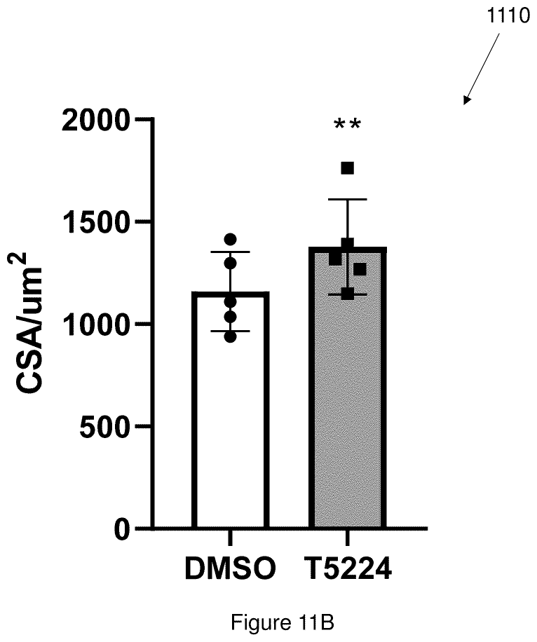

Turning to FIGS. 11A 1100 and 11B 1110, mice treated with the composition comprising a therapeutically effective amount of a compound of Formula (II) show healthy muscle morphology and increased muscle fibre size (18.8%), illustrated delayed muscle loss.

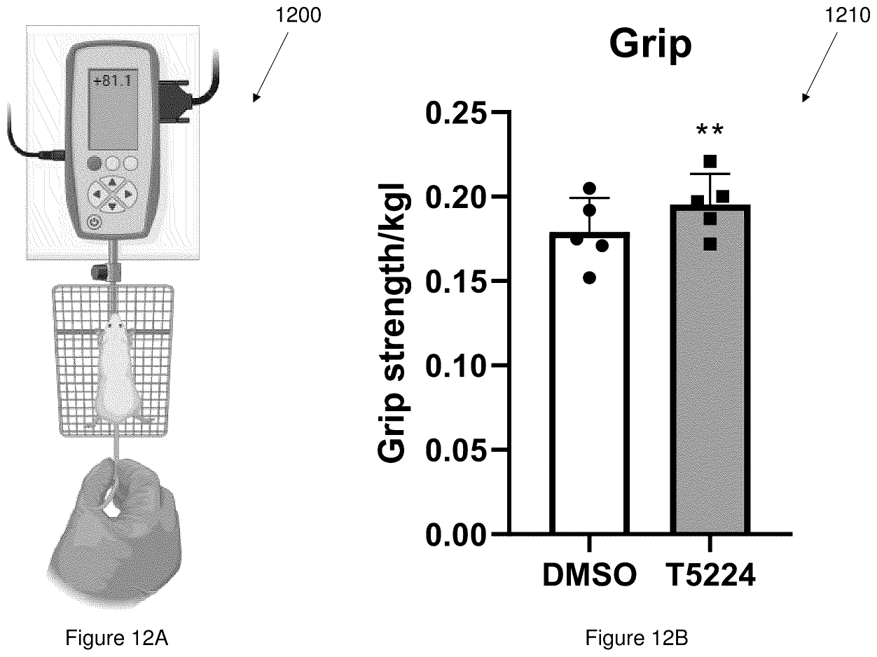

As shown in FIGS. 12A 1200 and 12B 1210, injection with a composition comprising a therapeutically effective amount of a compound of Formula (I) prior to treatment with the composition comprising a therapeutically effective amount of a compound of Formula (II) resulted in increased muscle strength. The schematic of FIG. 12A 1200 shows the experimental testing of grip strength in the tested mice. Muscle function was significantly enhanced following treatment with the composition comprising a therapeutically effective amount of a compound of Formula (II) as evidenced by a notable increase in grip strength (9.2%) (FIG. 12B 1210).

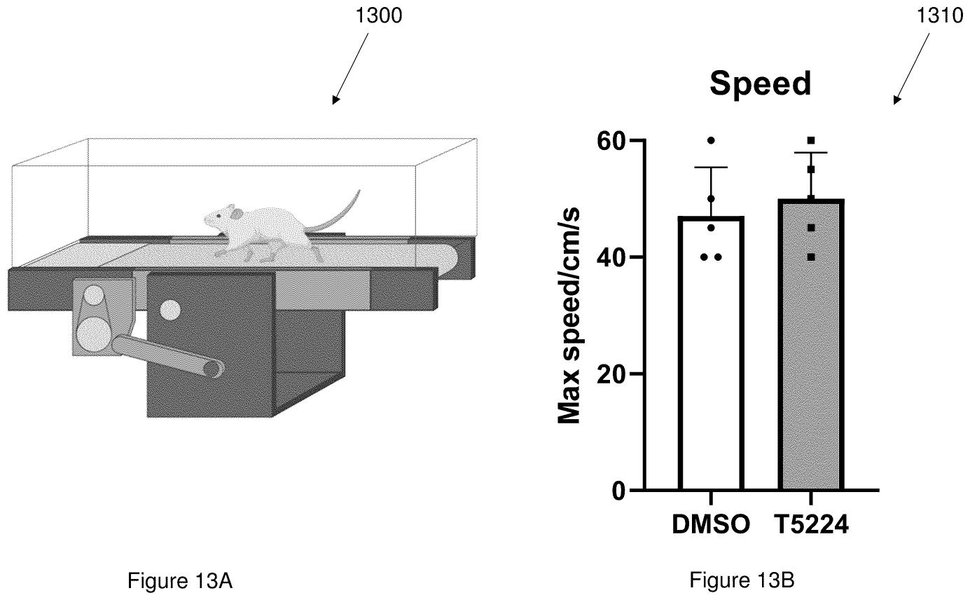

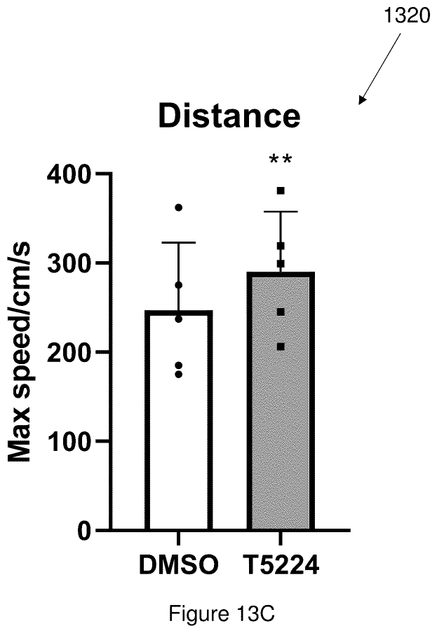

With reference to FIGS. 13A 1300, 13B 1310, and 13C 1320, when subjected to a treadmill running test, as illustrated in FIG. 13A 1300, in which the mice were adapted to a treadmill followed by a stepwise increase of running speed until exhaustion, the mice treated with the composition comprising a therapeutically effective amount of a compound of Formula (I) followed by treatment with the composition comprising a therapeutically effective amount of a compound of Formula (II) demonstrated a higher running speed (FIG. 13B 1310) (6.4%) and longer running distance (FIG. 13C 1320) (17.5%).

In a preferred embodiment, the composition comprising a therapeutically effective amount of a compound of Formula (II) targets c-Fos/activator protein (AP)-1 family. The composition targets one or more of JUNB, FOSL1, ATF6, FOSB, and JUND, resulting in delayed senescence. Preferably, the composition targets JUNB to delay cellular senescence.

In an alternative embodiment, the composition comprising a therapeutically effective amount of a compound of Formula (II) is administered in combination with a therapeutically effective amount of a compound of Formula (I), or a pharmaceutically acceptable salt, solvate, or hydrate thereof:

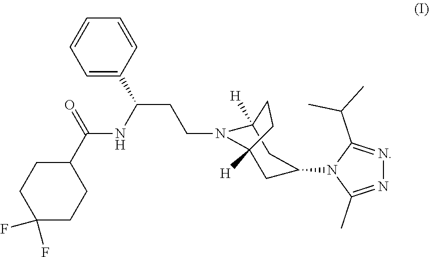 [SMILES: Cc1nnc(C(C)C)n1[C@@H]1C[C@H]2CC[C@@H](C1)N2CC[C@H](NC(=O)C1CCC(F)(F)CC1)c1ccccc1]

The therapeutically effective amount of the compound of Formula (II) administered is determined based on an assessment of degree of the muscle degradation in the subject and a severity of cellular senescence in the subject.

For example, the composition is formulated for administration in the subject via oral, injectable, or topical routes, and may be administered in combination with one or more therapeutic agents or supplementary treatments targeting muscle regeneration or senescence, including muscle growth stimulants, anti-inflammatory agents, other senotherapeutic compounds, or a combination thereof.

Example embodiments are also directed to the use of the compound of Formula (II), or a pharmaceutically acceptable salt, solvate, or hydrate thereof, in the manufacture of a medicament for the treatment of muscle degradation in a subject. For example, the medicament reduces or delays cellular senescence and a therapeutically effective amount of the medicament is administered to mitigate muscle degradation and improve muscle function. For example, the muscle degradation is associated with sarcopenia, muscle wasting disorders, age-related muscular weakness, or a combination thereof.

Preferably, the medicament targets c-Fos/activator protein (AP)-1 family and is formulated to modulate JUNB, FOSL1, ATF6, FOSB, and JUND in muscle cells. Most preferably, the medicament targets JUNB to delay cellular senescence.

In an example embodiment, the medicament targets genes, proteins, or signalling pathways involved in one or more of cellular senescence, inflammation, muscular degradation, sarcopenia, neurodegenerative diseases, cardiovascular ageing, or age-related diseases.

In another example embodiment, the medicament further comprises a therapeutically effective amount of a compound of Formula (I), or a pharmaceutically acceptable salt, solvate, or hydrate thereof:

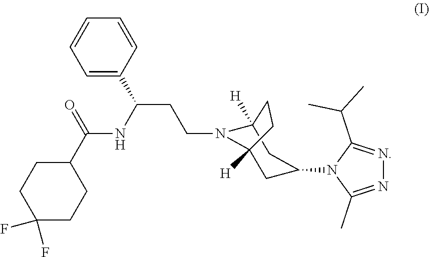 [SMILES: Cc1nnc(C(C)C)n1[C@@H]1C[C@H]2CC[C@@H](C1)N2CC[C@H](NC(=O)C1CCC(F)(F)CC1)c1ccccc1]

For example, the medicament is formulated as a dosage form selected from the group consisting of tablets, capsules, and powders. Further, the medicament may be formulated to deliver the compound of Formula (II) in an extended-release or controlled-release dosage form and may further comprise one or more pharmaceutically acceptable carriers or excipients.

Embodiments of the claimed invention advantageously provide a method of identifying senotherapeutic targets through the creation of a senescence atlas thus providing a means of examining how SASPs affect cellular interactions and tissue function that can be used for clinical exploitation. The claimed invention is also directed to a method of treating muscle degradation, the use of a medicament of a compound of Formula (I) in the manufacture of a medicament for the treatment of muscle degradation in a subject by targeting specific SASP factors, and the use of a medicament of a compound of Formula (II) in the manufacture of a medicament for the treatment of muscle degradation by targeting the AP-1 family, including JUNB.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

The experiments as described below provide further examples of the invention as claimed.

### EXAMPLES

### Example 1—Methods

### 1.1 Human Muscle Biopsy and Ethical Clearance

Hamstring muscle samples were collected during orthopaedic surgery with informed consent from 10 male patients in the Hong Kong cohort, with ages between 19-27 years old (young group) and 60-77 years old (aged group). Informed consent was obtained from the legally acceptable representative. Ethical approval was granted by the Joint Chinese University of Hong Kong-New Territories East Cluster Clinical Research Ethics Committee (2021.255-T). Exclusion criteria were myopathy, hemiplegia or hemiparesis, rheumatoid arthritis or other autoimmune connective tissue disorders, cancer, coronary heart disease, inability to consent, or major surgery in the previous 3 months.

### 1.2 Mice

C57BL/6 aged mice were purchased from Gempharmatech (Nanjing, Jiangsu, China). JunBf/f mouse strains were purchased from The Jackson Laboratory (Bar Harbor, ME, USA). The JunB-inducible conditional KO (iKO, Pax7CreERT2/R26Yfp; JunBf/f) strain and Ctrl (Pax7CreERT2/R26Yfp; JunB+/+) mice were generated by crossing Pax7CreERT2/R26Yfp mice with Junbf/f mice.

### 1.3 Animal Procedures

Inducible deletion of JunB was administered by intraperitoneal (IP) injection of tamoxifen (TMX) (Sigma-Aldrich, T5648) at 100 mg/kg (body weight). Maraviroc (Sigma-Aldrich, PZ0002-25 MG) treatment in aged C57BL/6 mice was administered by IP injection at 2 mg/kg (low dosage) or 10 mg/kg (high dosage) every two days for 3 months (short term) or 6 months (long term). For grip strength test, limb muscle grip strength of mice was measured by a grip strength meter (Kewbasis, KW-ZL-1) 3 times, the average values were calculated. For treadmill test, mice were adapted to a treadmill (Panlab, Harvard Apparatus, 76-0895) with a 5° incline at an initial speed of 10 cm/s, followed by a stepwise increase of 5 cm/s every two min until their exhaustion.

### 1.4 Cell Lines and Cell Culture

IMR-90 human fibroblast was purchased from the American Type Culture Collection (ATCC) and cultured in DMEM medium (Gibco, 12800-017) with 10% fetal bovine serum (Invitrogen, 16000044), 50 units/ml of penicillin and 50 μg of streptomycin (P/S, Gibco, 15140-122) at 37° C. in 5% CO2. Senescence was induced by culturing the cells with 50 μM Etoposide (ETO) for 2 days or passaged to the 10th generation. All cell lines tested negative for mycoplasma contamination.

### 1.5 Fluorescence-Activated MuSC Sorting and Culturing

Muscle stem cells, fibro-adipogenic progenitors and macrophages were sorted. Briefly, hindlimb muscles from mice and hamstring muscles from humans were digested with collagenase II (LS004177, Worthington, 1000 units per 1 ml) for 90 min at 37° C., the digested muscles were then washed in washing medium (Ham's F-10 medium (N6635, Sigma) containing 10% horse serum, heat-inactivated (HIHS, 26050088, Gibco, 1% P/S) before cells were liberated by treating with Collagenase II (100 units per 1 ml) and Dispase (17105-041, Gibco, 1.1 unit per 1 ml) for 30 min. The suspensions were passed through a 20G needle to release cells. Mononuclear cells were filtered with a 40 μm cell strainer and sorted by BD FACSAria IV with the selection of the GFP+ (MuSCs of Ctrl and Junb iKO mice); FITC−(CD45−, CD31−) APC−(SCA1−) PE+ (VCAM+)(MuSCs of young and aged mice); FITC−(CD45−, CD31−, CD34−) APC+ (CD29+) PE-CY7+ (CD56+)(MuSCs of human); FITC−(CD45−, CD31−, ITGA7−) APC+ (SCA1+) (FAPs); FITC−(Cd45−) APC−(Ly6G−) eFluor450+ (CD11b+) (MPs). Flowjo V10.8.1 was used for analysis of flow cytometry data. MuSCs were cultured in Ham's F10 medium with 20% FBS, 5 ng/ml β-FGF (PHG0026, Thermo Fisher Scientific) and 1% P/S, on coverslips and culture wells which were coated with poly-D-lysine solution (p0899, Sigma) at 37° C. overnight and then coated with extracellular matrix (ECM) (E-1270, Sigma) at 4° C. for at least 6 h.

### 1.6 Plasmids

pcDNA3.1-mouse-JunB plasmids were purchased from Youbio (http://www.youbio.cn/).

### 1.7 RNA Extraction and Real-Time PCR

Total RNAs were extracted using TRIzol reagent (Invitrogen) following the manufacturer's protocol. For quantitative RT-PCR, cDNAs were reverse transcribed using HiScript III First-Strand cDNA Synthesis Kit (Vazyme, R312-01). Real-time PCR reactions were performed on a LightCycler 480 Instrument II (Roche Life Science) using Luna Universal qPCR Master Mix (NEB, M3003L).

### 1.8 Immunoblotting, Immunofluorescence, and Immunohistochemistry

For Western blot assays, cultured cells were washed with ice-cold PBS and lysed in cell lysis buffer. Whole cell lysates were subjected to SDS-PAGE and protein expression was visualised using an enhanced chemiluminescence detection system (GE Healthcare, Little Chalfont, UK). The following dilutions were used for each antibody: JUNB (Cell Signalling Technology, #3753; 1:1000), Histone 3 (Santa Cruz, sc-56616; 1:5000). For SA-β-GAL staining, the β-galactosidase Senescence Kit (Cell Signalling Technology, #9860) was used. Briefly, cells were fixed for 15 mins followed by washing in PBS twice. Fixed cells were then incubated with β-galactosidase staining solution at 37° C. in a dry incubator (no CO₂) (overnight for MuSCs, 6 hours for IMR-90 cell). The cells were then observed and counted for the SA-β-GAL positive cells. For immunofluorescence staining, cultured cells were fixed in 4% PFA for 15 min and blocked with 3% BSA within 1 hour. Primary antibodies were applied to samples with indicated dilution below and the samples were kept at 4° C. overnight. For immunofluorescence staining, cultured cells or myofibers were fixed in 4% PFA for 15 min and permeabilised with 0.5% NP-40 for 10 min. Cells were then blocked in 3% BSA for 1 hour followed by incubation with primary antibodies overnight at 4° C. and secondary antibodies for one hour at RT. Finally, the cells were mounted with DAPI to stain the cell nucleus and images were captured by a Leica fluorescence microscope. Primary antibodies and dilutions were used as following: PAX7 (Developmental Studies Hybridoma Bank; 1:50), P16 (Abcam, ab211542, 1:200). For immunohistochemistry, in brief, slides were fixed with 4% PFA for 15 min at room temperature and permeabilised in ice-cold methanol for 6 min at −20° C. Heat-mediated antigen retrieval with 0.01 M citric acid (pH 6.0) was performed for 5 min in a microwave. After 4% BSA (4% IgG-free BSA in PBS; Jackson, 001-000-162) blocking, the sections were further blocked with unconjugated AffiniPure Fab Fragment (1:100 in PBS; Jackson, 115-007-003) for 30 min. The biotin-conjugated anti-mouse IgG (1:500 in 4% BBBSA, Jackson, 115-065-205) and Cy3-Streptavidin (1:1250 in 4% BBBSA, Jackson, 016-160-084) were used as secondary antibodies. Primary antibodies and dilutions were used as follows: Laminin (Sigma-Aldrich L9393-100UL, 1:800), P16 (Abcam, ab270058, 1:200), P21 (Santa Cruz Biotechnology, sc-6246, 1:200) PAX7 (Developmental Studies Hybridoma Bank; 1:50) for staining of muscle cryosections. Images were slightly modified with ImageJ in which background was reduced using background subtraction and brightness and contrast were adjusted. H&E (Hematoxylin and eosin), was performed.

1.9 Multiome snRNA-Seg/ATAC-Seq Profiling in Human Muscle

snRNA-seq/ATAC-seq and scRNAseq were performed on 10× genomics platform. Briefly, mononucleated resident cells were isolated from human muscle as described in “Fluorescence activated MuSC sorting and culturing” part with 7-AAD (Thermo Scientific, 00-6993-50) staining for viability selection. Red blood cells were eliminated by ACK buffers (150M NH4Cl, 100 mM KHCO3, 10 mM EDTA-2Na) before sorting. After sorting, live cells were washed with 0.04% BSA in PBS twice and resuspended in the BSA solution. For snRNA-seq/ATAC-seq, nuclei were isolated from the suspended cells according to the manufacturer's instruction CG000366. Rev D. Isolated nuclei were counted under a microscope and Typan blue was used to examine the number and integrity. The isolated nuclei were then resuspended at an appropriate concentration (5000-10000 nuclei/μl); Library construction was performed following the manufacturer's instructions for generation of Gel Bead-In Emulsions (GEMs) using the 10× Chromium system.

1.10 Initial Processing and Quality Control of snRNA-Seq and snATAC-Seq Data

Raw sequencing reads of human skeletal muscle were aligned to the pre-built reference on GRCh38 and counted using Cell Ranger ARC (version 2.0.1) with the default parameters. High-quality nuclei were kept based on gene expression data (>1000, and <40,000 UMI, and mitochondrial percent <20) and chromatin accessibility data (>1000, and <100,000 ATAC read counts). Seurat (version 5.0.1) object of each sample was constructed from clean nuclei.

### 1.11 Integration, Clustering and Identification of Cell Types

The inventors next performed pre-processing and dimensional reduction on both assays independently. First, the RNA count matrix of each sample was normalized using the SCTransform function with the mitochondrial percent variable regressed out. To match shared cell types across samples, features and anchors for downstream integration were selected with the FindIntegrationAnchors and IntegrateData functions, ensuring accurate comparative analysis. After data integration and scaling, principal component analysis (PCA) was conducted on a new integrated assay with the RunPCA function, and clustering and dimensionality reduction analysis was performed with the FindNeighbors, FindClusters, and RunUMAP functions. Cell types were identified and annotated according to the expression levels of the classic marker genes.

The marker genes of each cell type were calculated using the FindAllMarkers function with the cutoff of LogFC>1 and adjusted P values <0.05 using t-test.

The ATAC counts of each sample were normalized by RunTFIDF function. LSI coordinates at the sample level were computed on the normalized ATAC matrix using RunSVD. To identify integration anchors, different samples were projected into a shared space by FindIntegrationAnchors function and the low-dimensional cell embeddings (the LSI coordinates) across the datasets were integrated using the IntegrateEmbeddings function. UMAP clustering was created using the integrated embeddings by RunUMAP function.

A weighted nearest neighbor (WNN) graph was constructed by FindMultiModalNeighbors from a list of two-dimensional reductions: PCA from RNA assay and integrated LSI from ATAC assay. WNN graph was used for following UMAP visualization and clustering.

### 1.12 Transcriptional Noise Analysis

Transcriptional noise/heterogeneity analysis was conducted. To account for differences originating from UMI counts and cell-type composition in different age groups, all cells were down-sampled to a specified number of UMIs, cell numbers were then down-sampled so that equal numbers of young and aged cells were used. A list of representative invariant genes was selected to calculate the Euclidean distance from each cell to the corresponding cell-type mean vector within each age group. Additionally, the Euclidean distances were averaged for each donor from two aged groups to further remove technical confounding. The Euclidean distance was used to measure the transcriptional noise at both single-cell and cell-type levels.

### 1.13 Ageing Sensibility Analysis

The prioritisation of cell types in the response to human muscle ageing was calculated and named as Augur Score using calculate_auc function from Augur package (version 1.0.3) by inputting the genesby-cells scRNA-seq matrix and a data frame containing cell type and aged group columns.

### 1.14 Senescent Cell Identification

To define senescent cells, we developed a unified senescence scoring (USS) algorithm based on five established senescence gene databases (SM: SenMayo, CA: CellAge, GA: GenAge, and SE: Senescence Eigengene approach). All cells were first divided based on cell types. Within the same cell type, ss-GSVA score was calculated for each cell with GSVA package (version 1.42.0) using the four different gene sets. The inventors excluded known senescent markers (P16, P15, P19, P21, P27, and PAI-1) from all four gene lists, since these genes will serve as additional validation of senescence signatures in the detected cell subset. ss-GSVA score SM, CA, GA, and SE were each split into two halves by the median value and senescent cells were defined as those possessing ss-GSVA scores in the upper half level.

### 1.15 Differential Expression and Gene Ontology Enrichment Analysis

Differentially expressed genes (DEGs) were determined in senescent vs. non-senescent for each cell type. DEGs were analysed by FindMarkers function in Seurat using Wilcoxon Rank Sum test, and were detected with the cutoff of LogFC>0.5 and adjusted P value <0.05. Sn-DEG lists for each cell type. Gene Ontology (GO) enrichment analysis for Sn-DEG sets was performed with the enrichGO function in the clusterProfiler (version 4.2.0) package. GO biological terms with Benjamini-Hochberg adjusted P value (FDR)<0.05 were considered significantly enriched.

### 1.16 Cell Fate Trajectory Analysis

To infer the ageing pace for selected cell types, Monocle (version 2.14.0) package was used for cell trajectory and pseudotime analysis. Briefly, for each cell cluster from two age groups, the Sn-DEGs in the cell type were used as ordering genes for DDRTree analysis by reduceDimension function and pseudotime ordering by orderCells function. To identify genes with expression patterns positively or negatively linked to pseudotime scale, Spearman correlations between the pseudotime value and gene expression levels were calculated among cells clustered along the pseudotime trajectory. Genes with high correlation with pseudotime scale were visualized with smooth expression curves by plot_pseudotime_heatmap function in Monocle package.

For MuSCs, the cells falling into late 1 and late 2 branches were extracted, and DEGs between the two late fates were detected (late branch-DEGs). Furthermore, late branch-specific DEGs significantly linked to pseudotime at late branch stage were identified and visualized in a similar manner. To further examine the senescence characteristics along the pseudotime trajectory, cell cycle activity signature was defined as ss-GSVA score for cell cycle gene list from REACTOME knowledgebase.

### 1.17 Cell-Cell Communication Analysis

Cell-cell interactions were inferred by CellChat (version 1.6.1) based on the expression of known ligand-receptor pairs in various cell types. Cells from young and aged groups were applied to CellChat separately and merged into one CellChat object. Dysregulated signalling during ageing is identified by identifyOverExpressedGenes and identifyOverExpressedInteractions functions. Age group-specific pathways and ligand-receptor pairs were also detected and visualized using functions wrapped in CellChat.

### 1.18 DNA Sequence Motif Enrichment and TF Regulation Analysis

ATAC peaks were called for each cell type using Signac and used in subsequent analyses retaining the cell type annotations. To search and compute enriched motifs, the DNA sequence of each peak was scanned, and a motif object was created and added to the Seurat object. Per-cell accessibility scores for known motifs were calculated and stored as a new assay (chromvar) by RunChromVAR function wrapped in chromVAR package (version 1.16.0). The chromvar assay contained chromVAR motif accessibilities and facilitated the identification of regulators of senescent cell state. Putative TF regulators were defined as those with significantly higher accessibility scores in senescent compared to non-senescent cells by wilcoxauc function from presto package (version 1.0.0).

### 1.19 Transcriptional Regulatory Network Analysis

To infer putative peak-gene regulatory interactions from paired snATAC-seq and snRNA-seq data, the distal cis-regulatory elements significantly associated with genes were computed by FigR. DORC (domains of regulatory chromatin) analysis was conducted to assess the accessibility of peaks within a fixed window (100 kb) centred around the transcription start site (TSS) of specific target genes and correlated with their expression levels. By combining the significance estimates of relative motif enrichment and RNA expression correlation for a given DORC, a signed regulation score (RS) was calculated, with the sign indicating whether the TF acts as an activator or repressor. TF-gene networks were then inferred to pinpoint candidate TF regulators. Specifically, JUNB DORC accessibility was calculated as the accessibility of JUNB motif-containing peaks in each SASP target gene, to show that JUNB accessibility can predict SASP gene expression along senescence trajectories. To visualize dynamics of DORC accessibility and gene expression of JUNB-target SASP genes along the pseudotime axis, we used the genSmoothCurves function to fit smooth spline curves for JUNB DORC accessibility and gene expression matrix dynamics along ageing pseudotime on a gene-wise basis. Subsequently, these matrices were normalized to the 1-99 percentile values respectively, to the relative difference between DORC and RNA. Furthermore, we applied a loess smoothing function to the normalized DORC/RNA values in relation to the smoothed aging pseudotime, which was then overlaid and visually represented.

1.20 scRNA-Seq RNA-Seq and Data Analysis

For single-cell RNAseq profiling in DMSO and MVC-treated mice, mononucleated cells were sorted as described in “Fluorescence-activated MuSC sorting and culturing” part with 7-AAD (Thermo Scientific, 00-6993-50) staining for viability selection. After sorting, cells were resuspended in the BSA solution at an appropriate concentration (800-1200 cells/μl). Suspended cells were counted under a microscope and Typan blue was used to examine the cell viability. Library construction was performed following instructions for generation of Gel Bead-In Emulsions (GEMs) using the 10× Chromium system. To analyse the above generated scRNA-seq data, cells with low gene number (fewer than 500) and high ratio of mitochondrial genes (more than 10%) were first removed. SCTransform normalization, clustering, and cell-cell interaction analysis were conducted in a similar manner as the snRNA-seq data analysis described above.

### 1.21 Bulk RNA-Seq and Data Analysis

For conducting RNA-seq (polyA+mRNA) in MuSCs from DMSO and MVC-treated mice, following protocol total RNAs were subjected to polyA selection (Ambion, 61006) followed by library preparation using NEBNext Ultra II RNA Library Preparation Kit (NEB, E7770S). Libraries were paired-end sequenced with read lengths of 150 bp on Illumina Nova-seq S4 instruments. The raw reads of RNA-seq were processed following the procedures described. Briefly, the adapter and low-quality sequences were trimmed from 3′ to 5′ ends for each read, and the reads shorter than 36 bp were discarded. The clean reads were aligned to mouse (mm10) reference genome with STAR. Next, Cufflinks was used to quantify the gene expression. Genes with expression level change >1.5-fold and adjusted p-value <0.1 were identified as DEGs between two conditions. GO enrichment analysis was performed using R package clusterProfiler.

### 1.22 CUT&RUN-Seq and Data Analysis

CUT&RUN assay was conducted following the inventors' protocol using 200,000 MuSCs or IMR-90 cells with the CUT&RUN assay kit (Cell Signalling Technology, 86652). In brief, FISCs were harvested and washed by cell wash buffer, then bound to concanavalin A-coated magnetic beads. Digitonin Wash Buffer was used for permeabilization. After that, cells were incubated with 2 μg of JUNB antibody (Cell Signalling Technology #3753) or H3K27ac (Cell Signalling Technology #8173) overnight at 4° C. with shaking. Then, cell bead slurry was washed with Digitonin Wash Buffer and incubated with Protein A-MNase for 1 hr at 4° C. with shaking. After washing with Digitonin Wash Buffer, CaCl₂ was added into the cell-bead slurry to initiate Protein A-MNase digestion, which was then incubated at 4° C. for half an hour. Then 2× Stop Buffer was added to the reaction to stop the digestion. CUT&RUN fragments were released by incubation for 30 min at 37° C. followed by centrifugation. After centrifugation, the supernatant was recovered, and DNA purification was performed using Phenol/Chloroform. For DNA library construction, a NEBNext® Ultra™ II DNA Library Prep Kit for Illumina® (NEB, E7645S) was used according to the instructions. Bioanalyser analysis and qPCR were used to measure the quality of DNA libraries including DNA size and purity.

The obtained sequencing raw reads were first pre-processed by quality assessment, adapters trimming, and low-quality filtering, and then were aligned to the mouse reference genome (mm10) using Bowtie2, and only non-redundant reads were kept. JUNB binding sites (JUNB peaks) were identified with p-value cutoff as 0.001 by MACS2 and genome browser visualization files were generated by Homer. To further investigate the JUNB binding sites, the inventors used H3K27ac signal to indicate enhancer regions using the H3K27ac CUT&RUN-seq data in mouse MuSC and published H3K27ac ChIP-seq data in IMR-90 cells from UCSD Human Reference Epigenome Mapping Project. Furthermore, to accurately link enhancers with their potential functioning genes, the inventors identified E-P interactions by analysing chromatin loop files sourced from IMR-90 eHi-C data. The inventors compared the JUNB binding sites between the aged vs. young and ETO vs. Ctrl groups, specifically focusing on the overlap with promoters and enhancers.

### 1.23 Statistics and Reproducibility

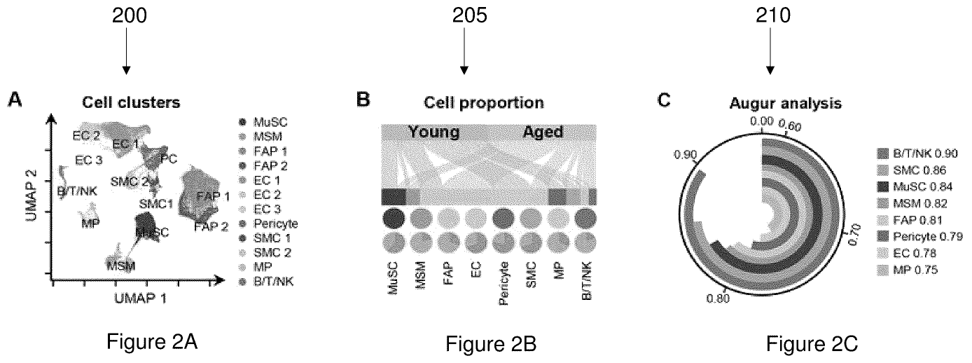

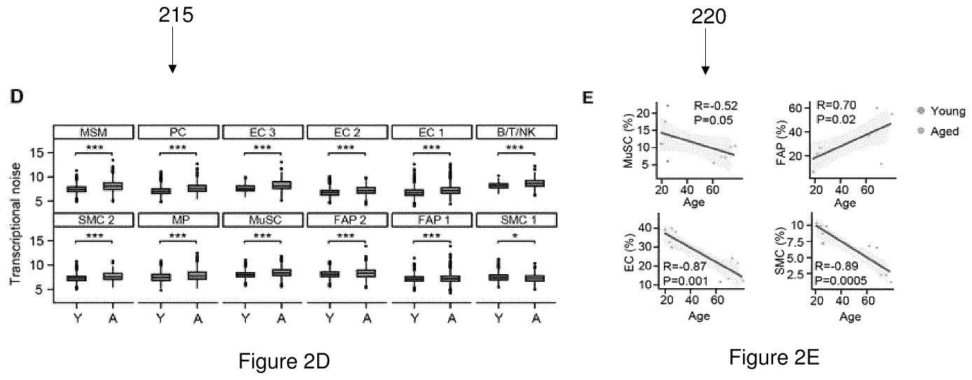

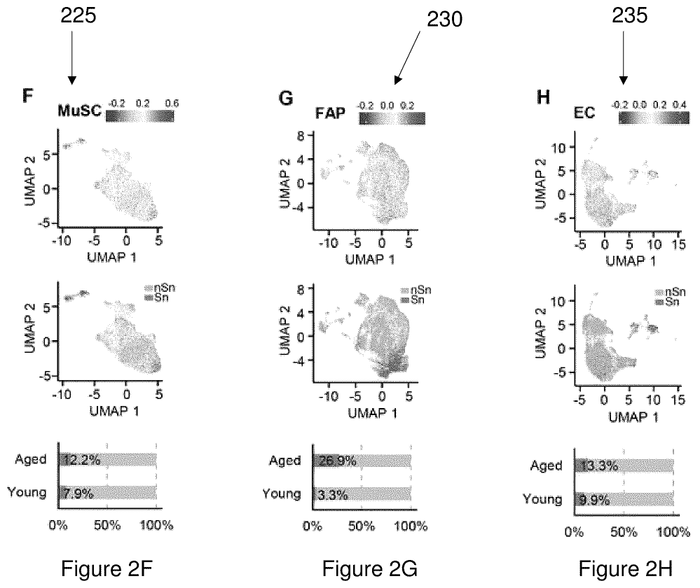

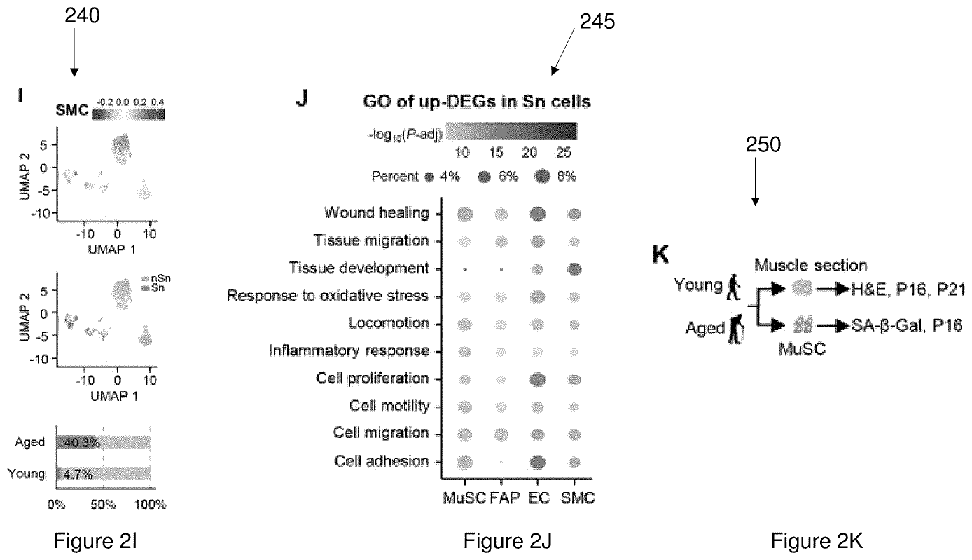

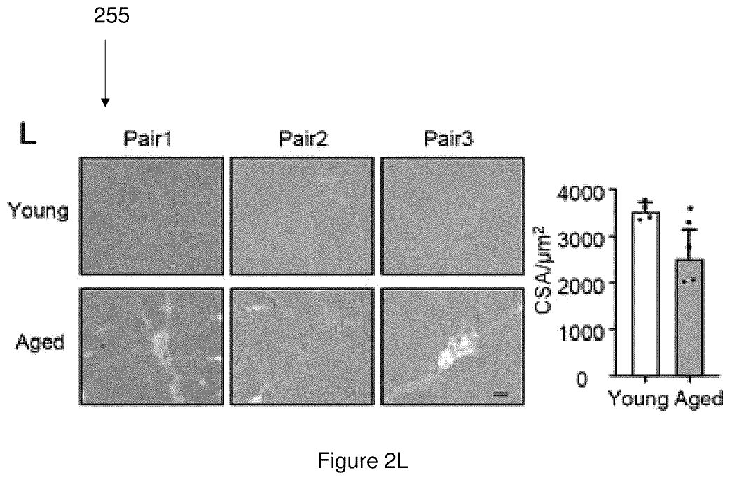

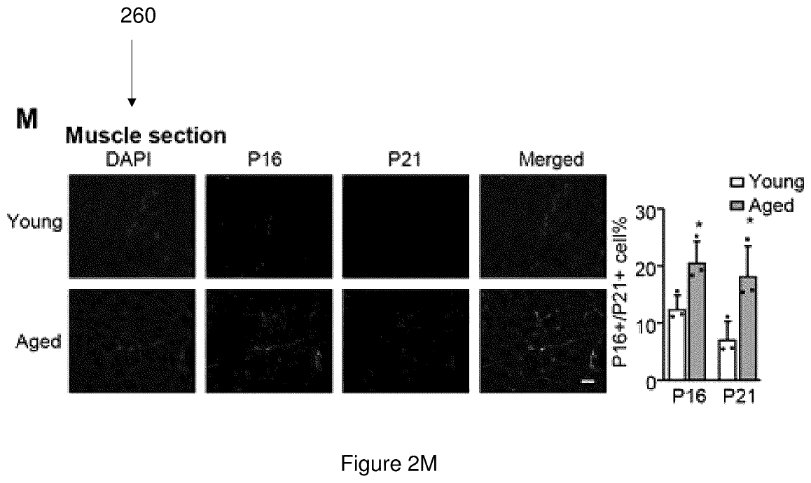

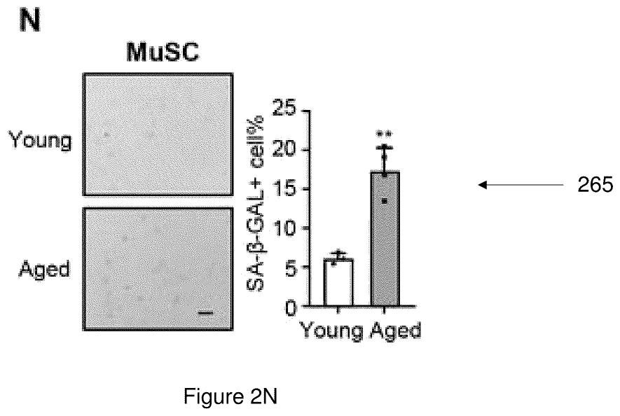

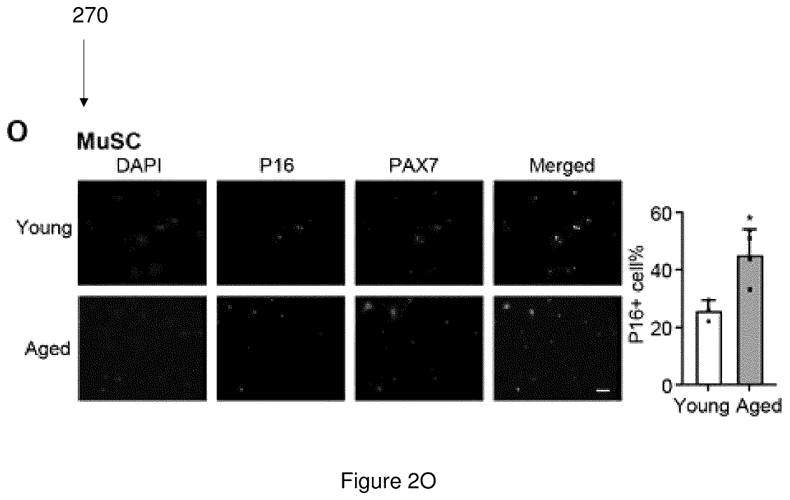

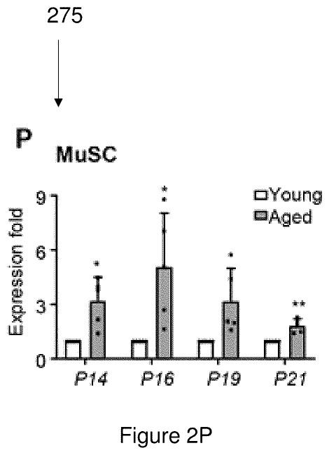

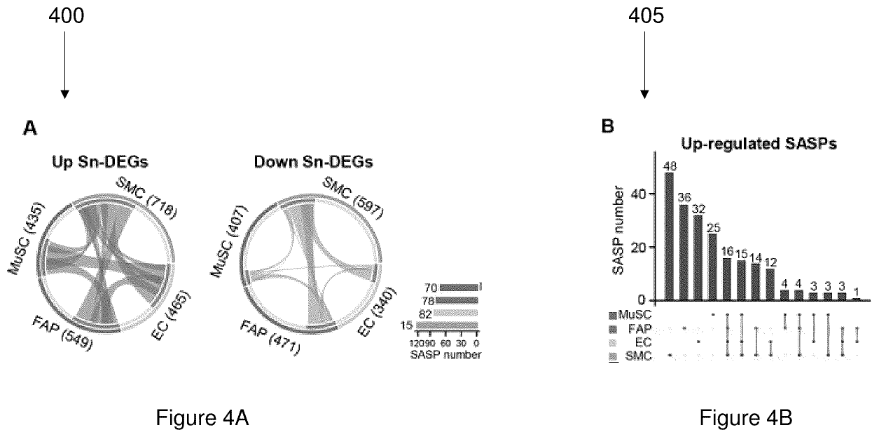

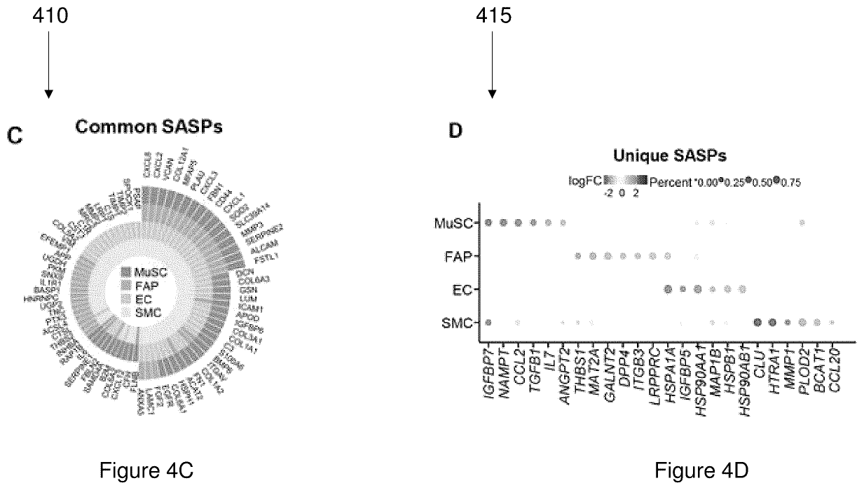

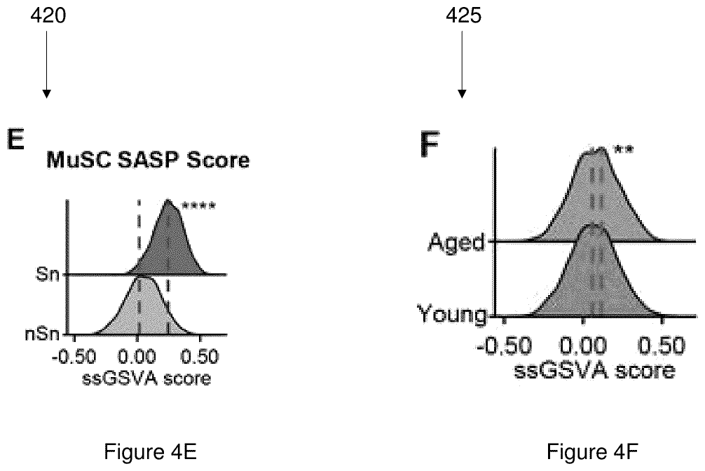

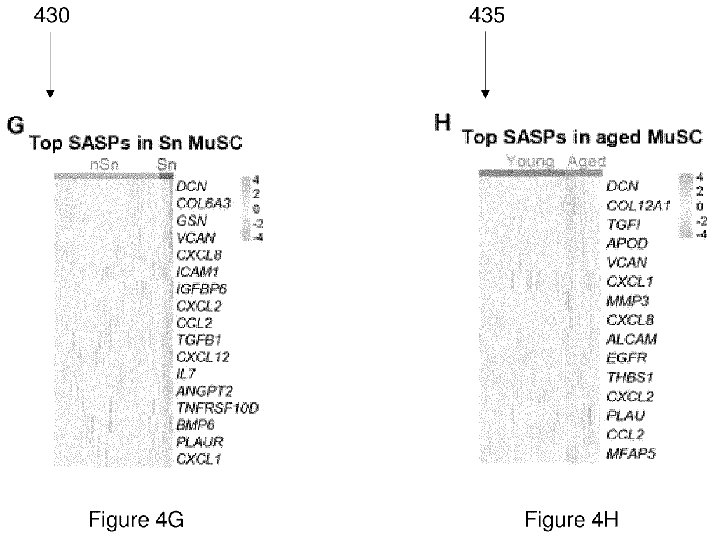

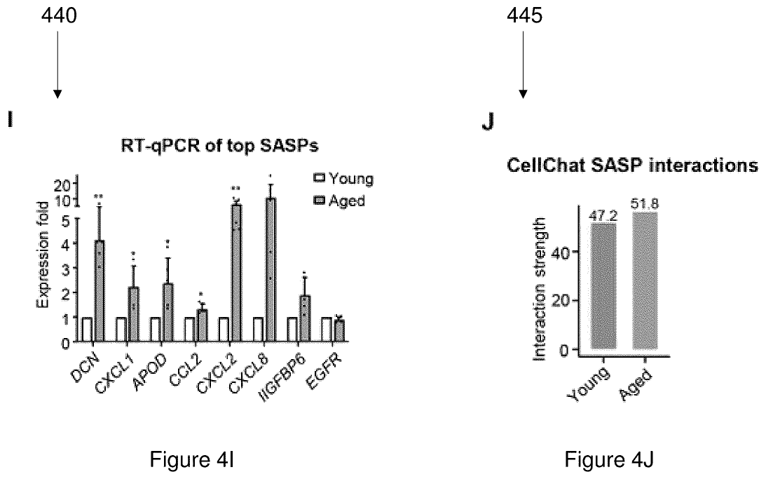

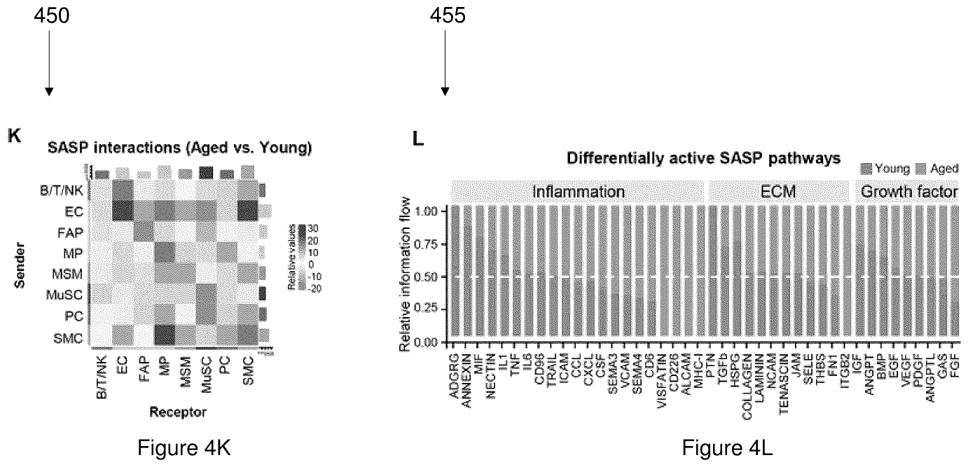

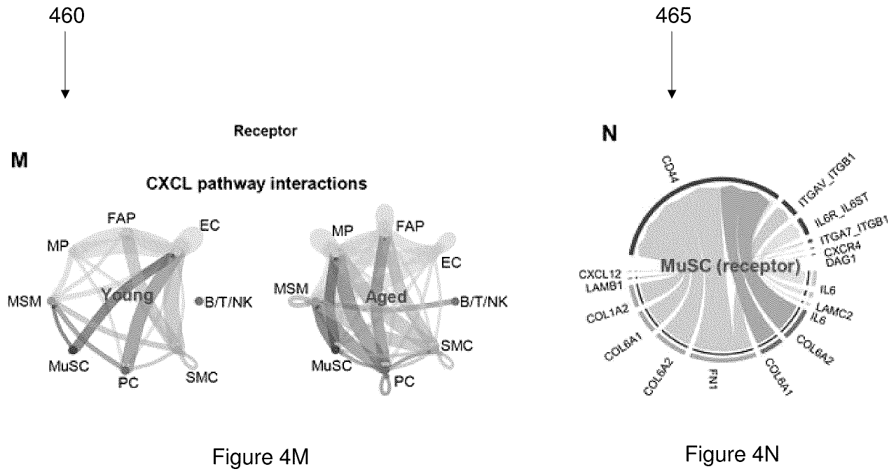

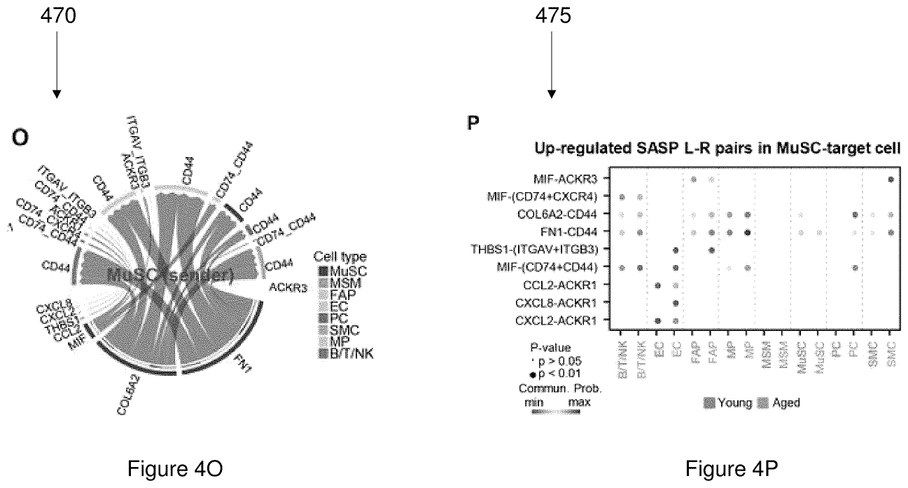

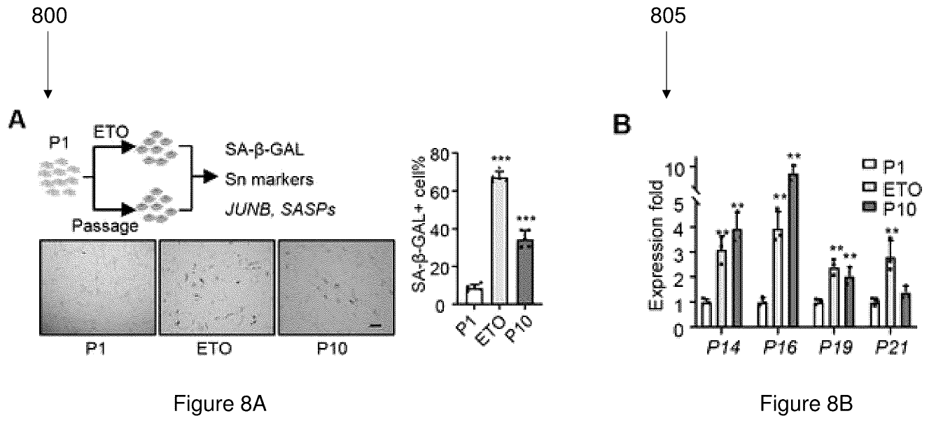

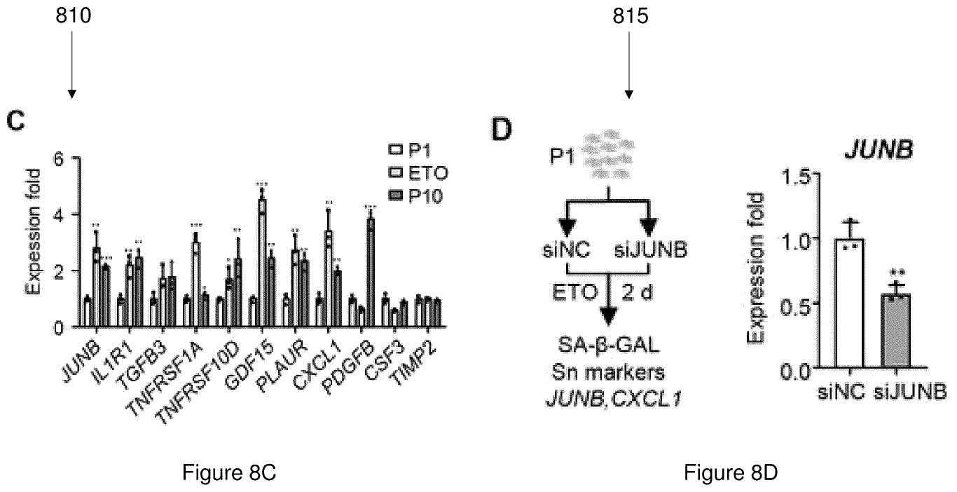

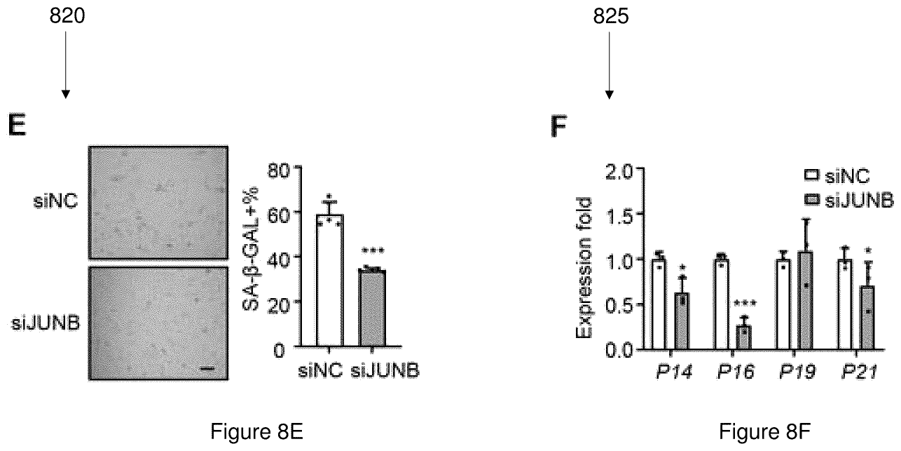

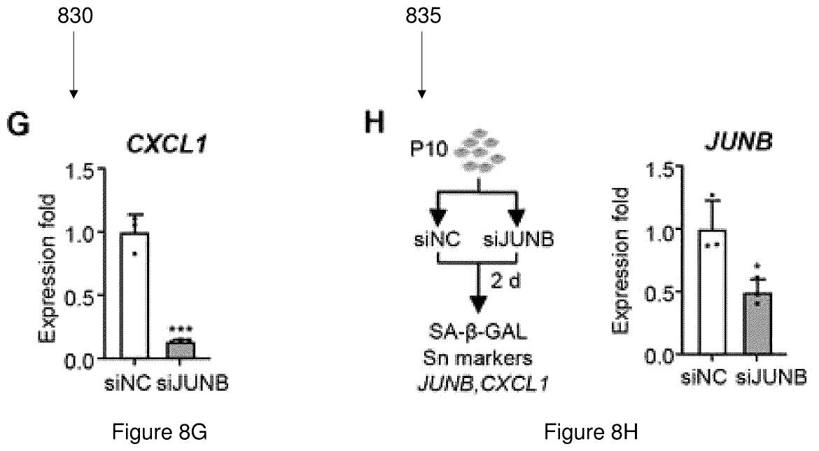

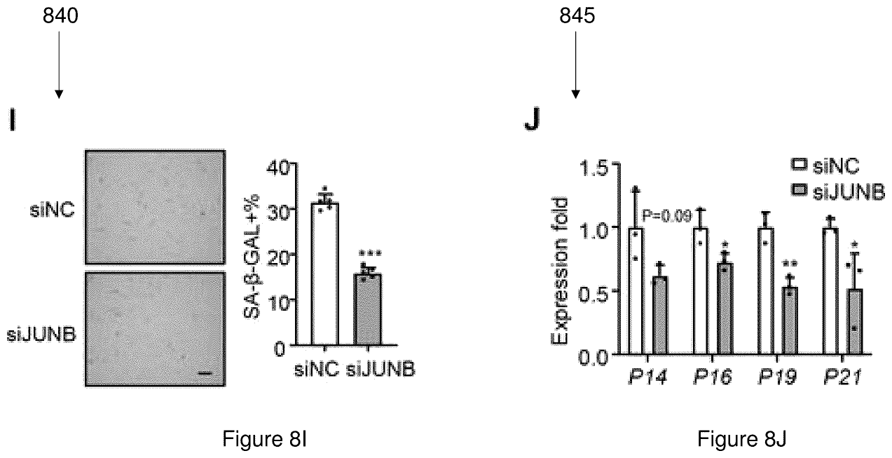

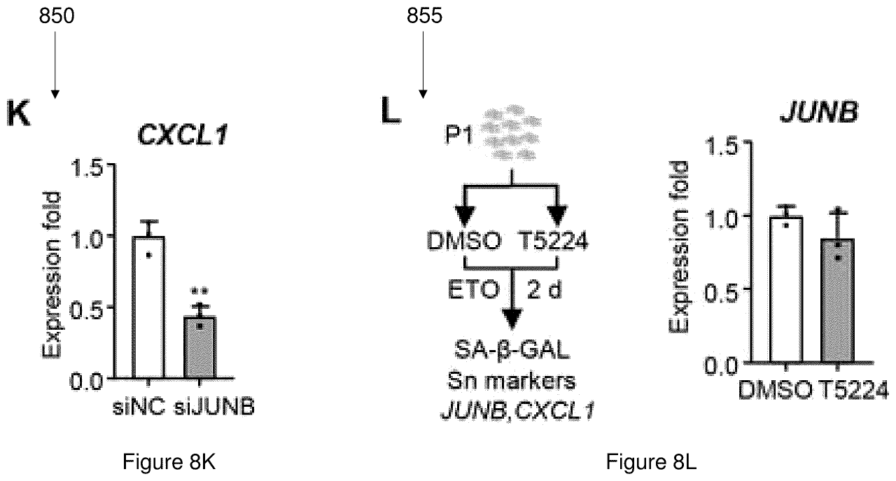

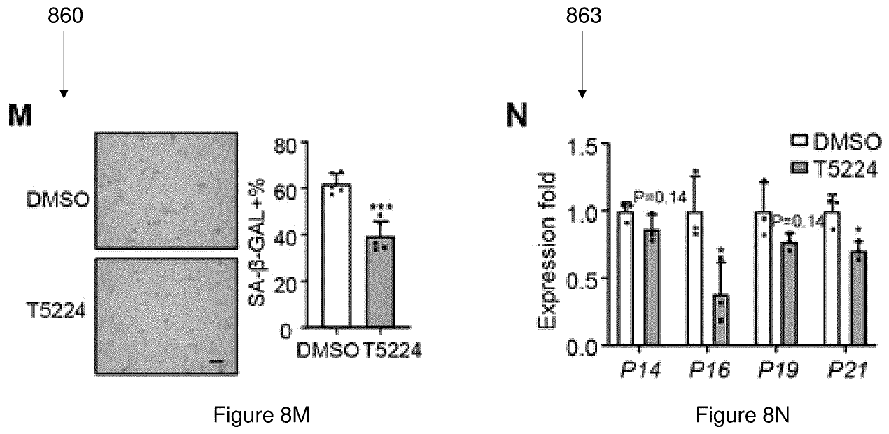

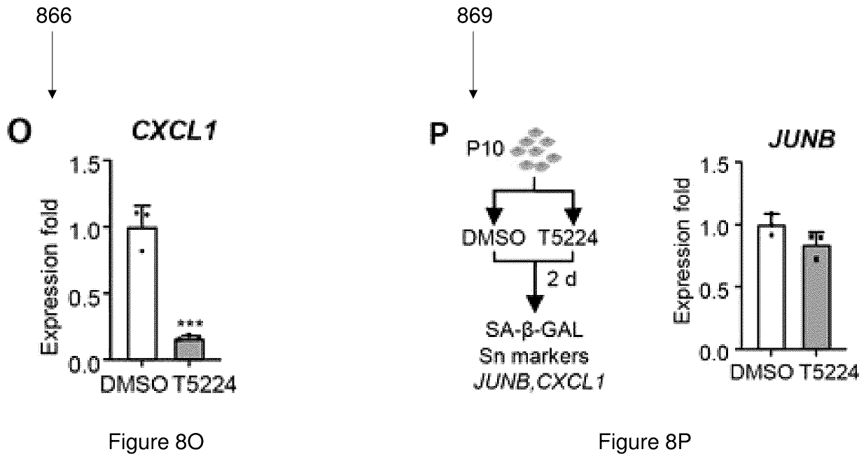

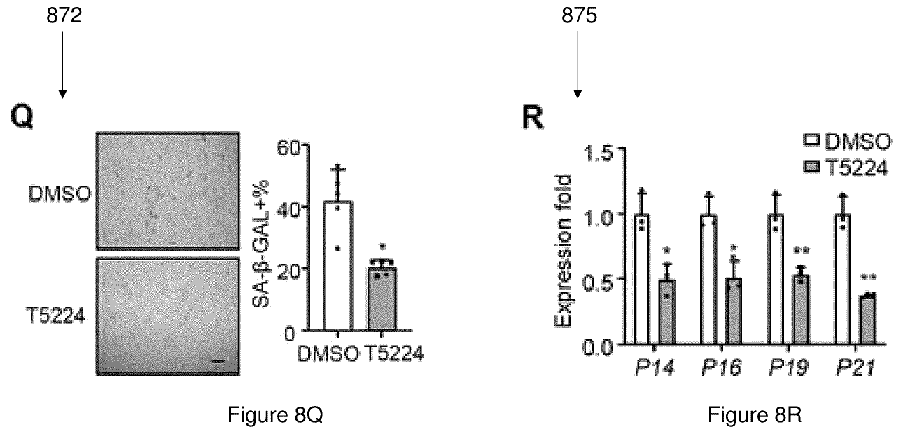

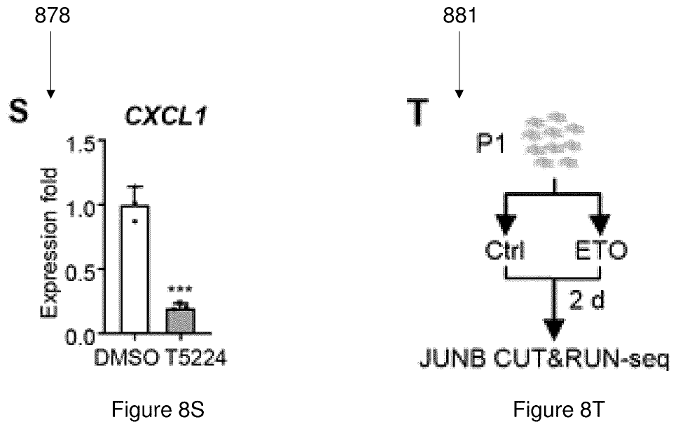

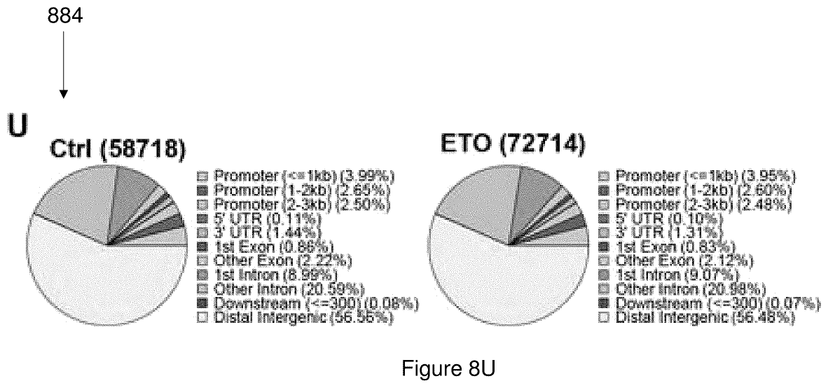

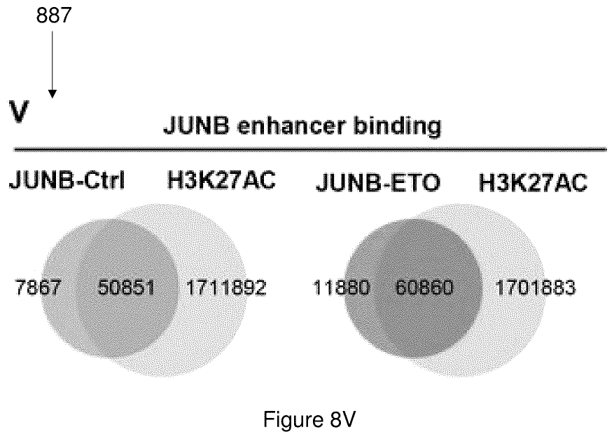

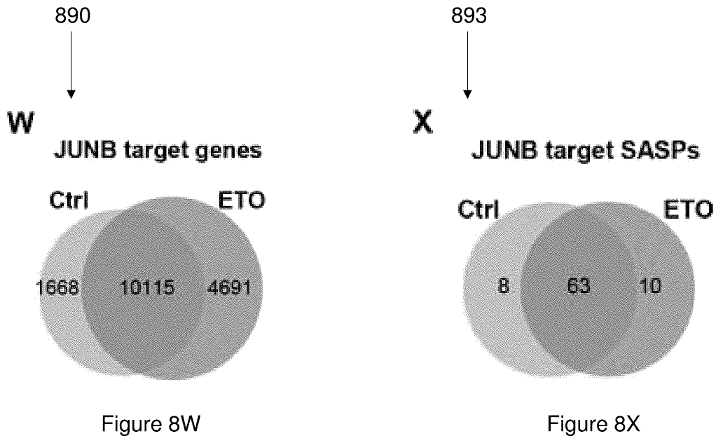

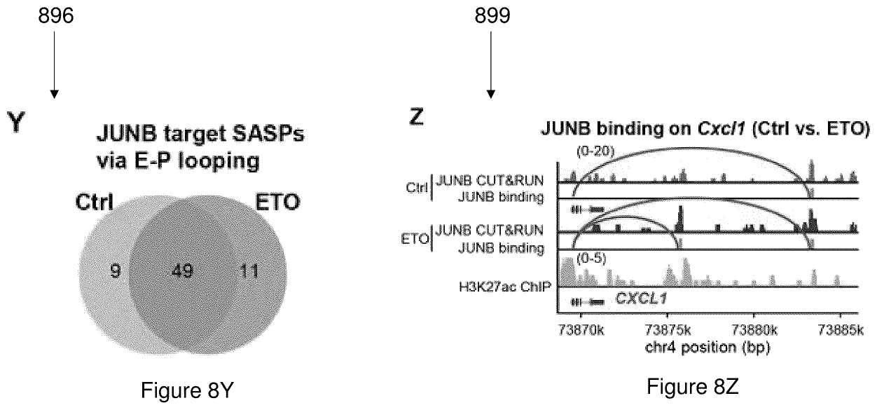

Data represents the average of at least three independent experiments, humans or mice+s.d. unless indicated otherwise. The statistical significance of experimental data was calculated by the Student's t-test (twosided). *p<0.05, **p<0.01, ***p<0.001, ****p<0.0001 and n.s.: no significance (p≥0.05). The statistical significance for the assays conducted with MuSCs from the same human or mouse with different treatments was calculated using the student's t-test (paired). *p<0.05, **p<0.01, ***p<0.001, n.s.=no significance (p≥0.05). Specifically, a single zero-truncated negative binomial distribution was fit to the input data and each region was assigned a P value based on the fitted distribution. Representative images of at least three independent experiments are shown in FIG. 2L 255, FIG. 2M 260, FIG. 2N 265, FIG. 2O 270, FIG. 5E 520, FIG. 4B 405, FIG. 4C 410, FIG. 4D 415, FIG. 4E 420, FIG. 4F 425, FIG. 8A 800, FIG. 8E 820, FIG. 8I 840, FIG. 8M 860, FIG. 8Q 872.

### 1.24 Data Availability

The human skeletal muscle single-nucleus multiome and CUT&RUN datasets within this study have been deposited in the Gene Expression Omnibus database under the accessions GSE268953 and GSE268431, respectively. The mouse single cell RNA-seq, bulk RNA-seq, and CUT&RUN datasets have been deposited under the accessions GSE268407, GSE268952, and GSE268433, respectively. All other data supporting the findings of this study are available from the corresponding author on reasonable request.

### Example 2—Results

### 2.1 Multiomics Mapping of Senescence Atlas in Human Muscle

To gain the first senescence blueprint of mononucleated cells in aging human muscle, the inventors obtained hamstring muscles from biopsies of 10 male donors: five young (19-27 years-old males who underwent anterior cruciate ligament reconstruction) and five aged (60-77 years-old males who underwent knee replacement surgery) (FIG. 1B 110) Mononuclear cells were FACS isolated and single nuclei were prepared for single-nucleus (sn) multiomics (simultaneous RNA-seq and ATAC-seq in one cell) analysis using a 10× Genomics Chromium. After stringent quality control, a total of 52,934 (30,390 from five young and 22,544 from five aged donors) qualified nuclei were obtained for downstream analysis. The snRNA-seq data quality, indicated by the number of read counts (UMI) and genes with at least one read count was highly correlated across all five donors; similar observation was made on the ATACseq data quality as indicated by ATAC read counts and ATAC peaks for each modality. Using unbiased clustering and uniform manifold approximation and projection (UMAP) analysis, 12 clusters of muscle resident mononuclear cells were identified with distinct transcriptomic and epigenomic signatures, including MuSCs, FAP (Fibro-adipogenic progenitors) 1, FAP 2, EC (endothelial cells) 1, EC 2, EC 3, Pericytes, SMC (smooth muscle cells) 1, SMC 2, MPs (macrophages), and B/T/NK (B-cells, T-cells and Natural Killer cells), along with a small number of nuclei from mature skeletal muscles (MSMs) despite terminally differentiated and post-mitotic myofibers were excluded from the procedure (FIG. 2A 200). The marker genes were used to annotate these clusters, for example, MYF5 and PAX7 for the MuSCs, DCN, FBN1 for the FAPs, ACTA1 for the ECs, and C1QA for the MPs. Strong correlations in global gene expression were observed across all 10 donors within each cell type suggesting that the within-group samples were highly comparable.

Consistently, all the expected cell types were identified among donors in each age group with comparable relative abundance. Decreased numbers of MuSCs, ECs, SMCs, and Pericytes were observed while the numbers of MSCs, FAPs, MPs, and B/T/NK cells increased in the aged compared to the young muscles (FIG. 2B 205), confirming the altered cellular composition and niche microenvironment during muscle ageing. The cell type prioritisation score analysis using Augur pinpointed B/T/NK cells (0.90), SMCs (0.86), MuSCs (0.84), and MSMs (0.82) as the cells most responsive to aging (FIG. 2C 210). Furthermore, by quantifying transcriptional noise, the inventors found most cells showed an elevated transcriptional heterogeneity among individual nuclei (FIG. 2D 215). This finding was further validated by calculating the average transcriptional noise scores per donor and an elevated ratio of transcriptional noise was observed in the aged group. For the four major non-immune cell types, MuSCs, ECs, FAPs and SMCs, correlation analysis demonstrated that the cell proportions of MuSCs, ECs, and SMCs decreased, and FAPs increased with ageing (FIG. 2E 220).

Next, to define the senescence atlas, the inventors calculated a unified senescence score (USS) by integrating four senescent gene sets (SenMayo, CellAge, GenAge, and Senescence Eigengene) through single-sample Gene Set Enrichment Analysis (GSEA). As a result, increased percentages of senescent cells were detected in all four cell types in aged vs. young muscle: MuSCs (12.2% vs. 7.9%, FIG. 2F 225), FAPs (26.9% vs. 3.3%, FIG. 2G 230), ECs (13.3% vs. 9.9%, FIG. 2H 235) and SMCs (40.3% vs. 4.7%, FIG. 2I 240). The inventors found that Gene Ontology (GO) enrichment analysis revealed that compared to the non-senescent (nSn) cells, the senescent (Sn) cells were enriched for pathways such as “wound healing”, “response to oxidative stress”, “cell adhesion” and “cell migration” etc. (FIG. 2J 245). The above findings from analysing the snRNA-seq data were further validated by additional experimentation (FIG. 2K 250). H&E staining of the muscle sections from three additional pairs of donors revealed decreased fiber size and increased inflammation in aged vs young muscles (FIG. 2L 255); elevated staining of P16 and P21 proteins was also detected (FIG. 2M 260). Moreover, MuSCs were freshly isolated by FACS and a significant increase of SA-β-GAL+ cells (FIG. 2N 265) and P16+ cells (FIG. 2O 270) were detected in aged muscles, accompanied by higher mRNA levels of senescence markers, P14, P16, P19 and P21 (FIG. 2P 275), thus affirming the inventors finding of increased senescence in aged MuSCs.

### 2.2 Heterogeneity and Dynamics of Cellular Senescence in Human Muscle

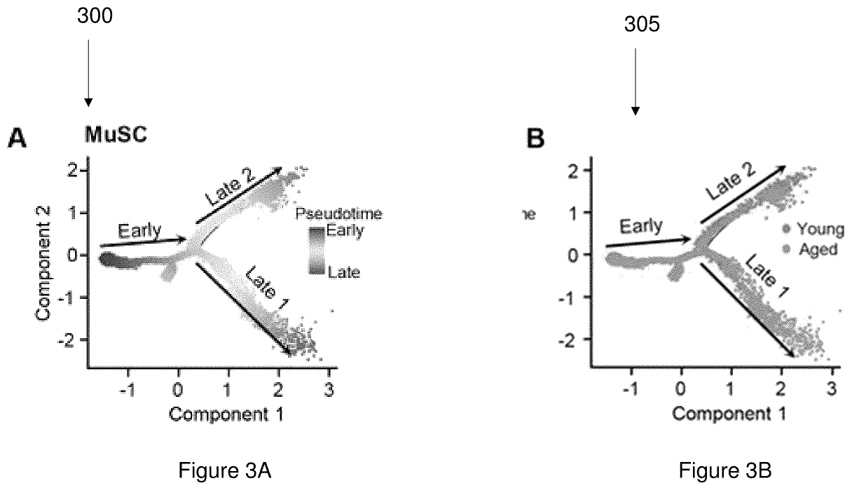

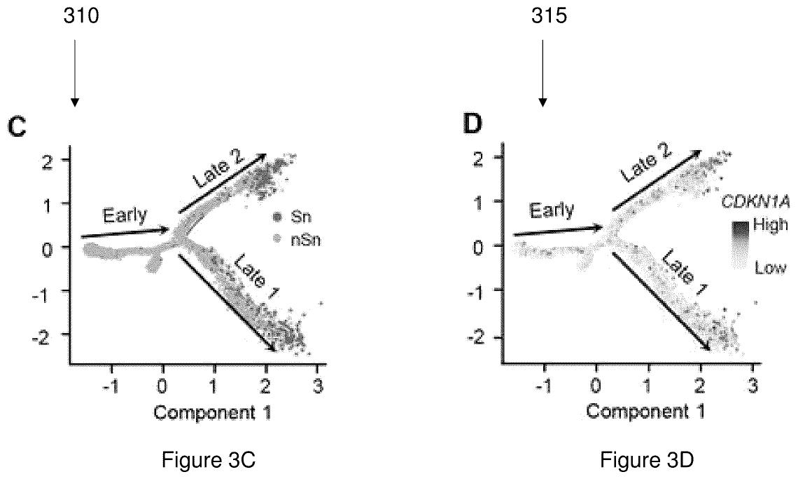

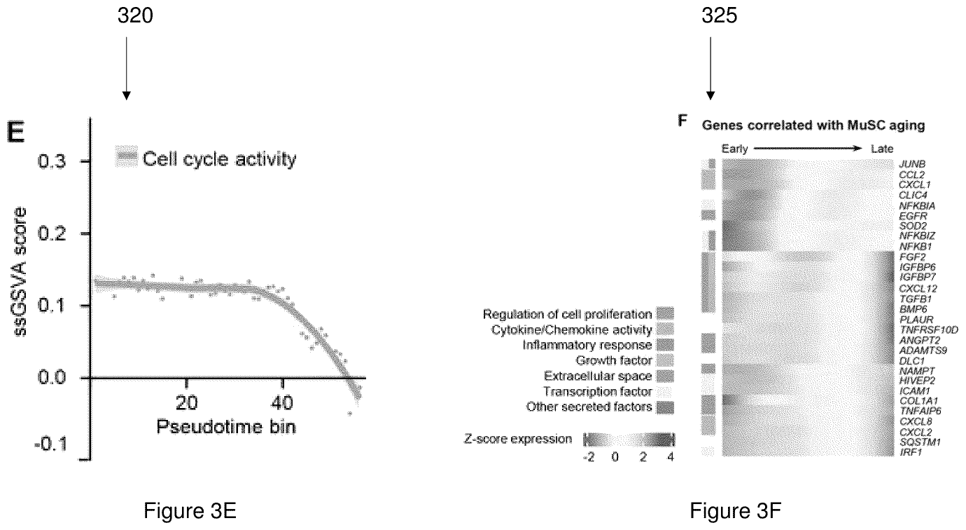

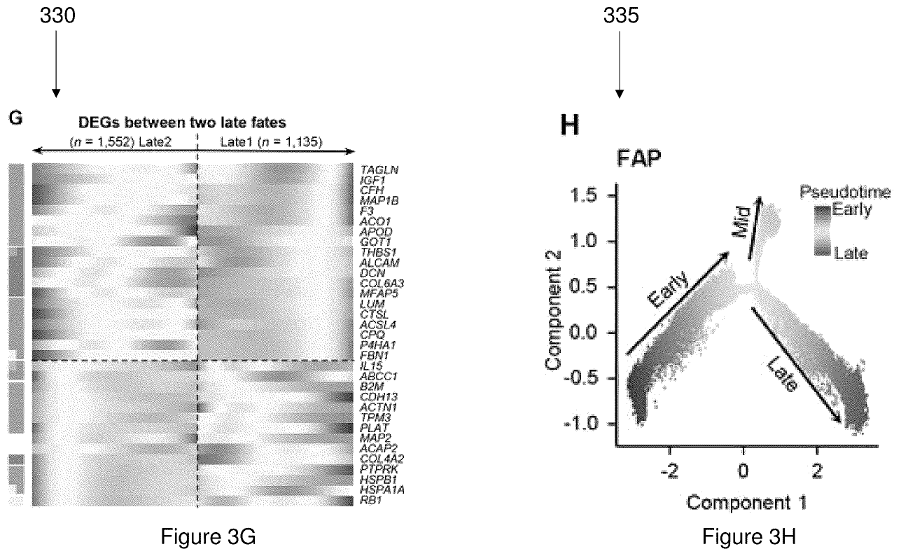

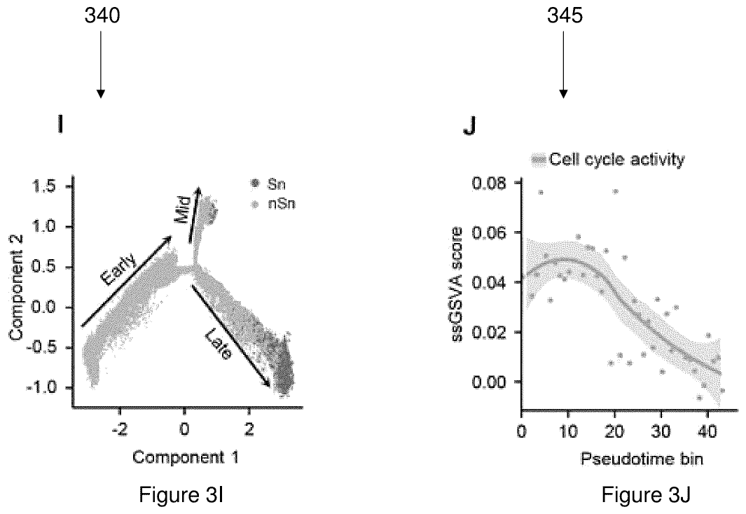

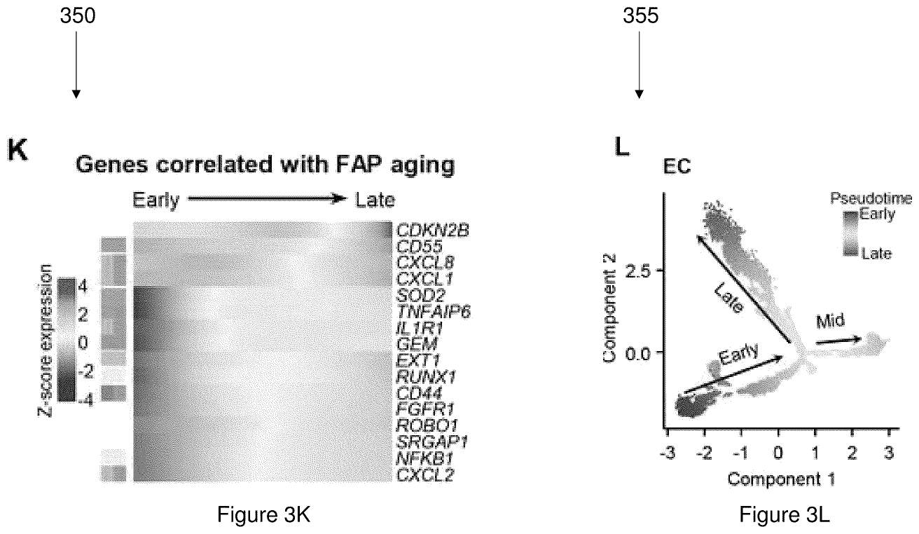

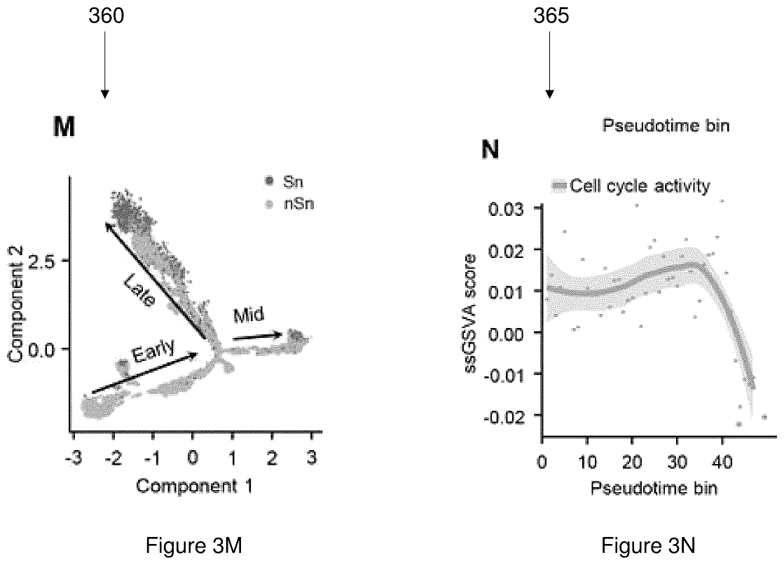

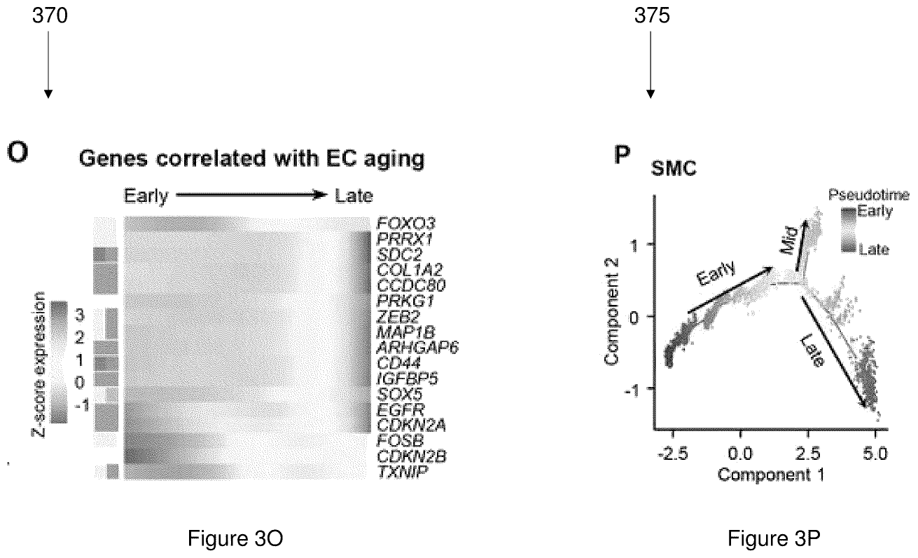

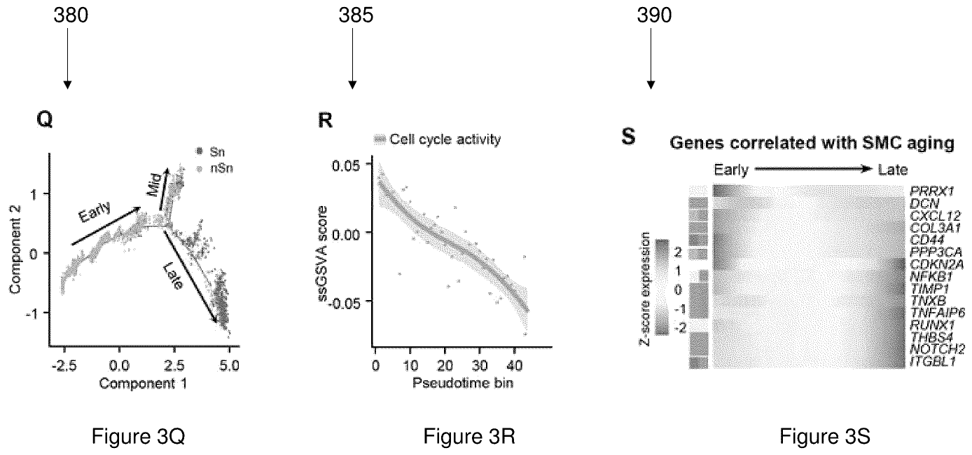

To further characterize MuSC senescence, pseudotime trajectory was utilized to reveal the MuSC fate which diverged into two late paths (FIG. 3A 300). The Early-Late 1 fate accurately captured the transition from young to aged nuclei, showing a significant increase in the proportion of aged nuclei along the pseudotime (FIG. 3B 305). Senescent MuSCs accumulated in both late branches (FIG. 3C 310) with CDKN1A gene highly expressed at the ends (FIG. 3D 315). The inventors then aggregated the expression of published cell cycle gene set of Reactome collection along the pseudotime trajectory and calculated a module score by ss-GSVA method; a decreased ss-GSVA score was observed over the trajectory (FIG. 3E 320), in agreement with cell cycle arrest in senescent cells. Differentially expressed gene (DEG) analysis uncovered various senescence-related GO terms such as “Extracellular space” (such as FGF2, IGFBP6, IGFBP7, CXCL12, TGFB1, BMP6), “Cytokine/chemokine activity” (such as CCL2, CXCL12, CXCL8, CXCL2), “Growth factor” (such as FGF2, IGFBP6, IGFBP7) etc. enriched in the late-phase MuSCs (FIG. 3F 325). Moreover, the DEGs in the Late 1 and Late 2 branches were enriched for distinct GO terms with Late 1 being highly pro-fibrotic compared to Late 2 (FIG. 3G 330). The inventors' above findings thus illustrate the high level of heterogeneity in senescent MuSCs.

The pseudotime trajectories for other cell types were also examined. FAPs displayed a more continuous and smooth cell fate trajectory (FIG. 3H 335), along which the ratio of aged nuclei significantly increased and senescent FAPs accumulated near the end (FIG. 3I 340), accompanied by a decreased cell cycle ss-GSVA score (FIG. 3J 345). Inflammatory response genes such as CDKN2B, CD56, CXCL8 and CXCL1 were among the most enriched pseudotime-correlated DEGs in the late stage (FIG. 3K 350). Similar to FAPs, ECs exhibited a relatively simple trajectory pattern, with cells starting from early-end, going through a middle branch, then falling into the late end (FIG. 3L 355). Mapping of ECs along the trajectory also uncovered a significant increase of aged nuclei, senescent ECs (FIG. 3M 360) and reduced cell cycle ss-GSVA score (FIG. 3N 365). Ageing ECs were enriched for genes associated with “Extracellular space” (such as COL1A2, CCDC80, PPKG1) and “Transcription factor” (such as ZEB2, MAP1B) (FIG. 3O 370). In the trajectory for SMCs, we observed a mixture of young and aged nuclei in the early end and middle phases, whereas the late end was predominantly occupied with aged nuclei. Most aged SMCs from the late end were detected as senescent and functionally distinct from the rest (FIGS. 3P 375-3Q 380). The inventors also observed a consistent decrease in cell cycle ss-GSVA score along the SMC pseudotime axis (FIG. 3R 385); and pseudotime-associated genes were enriched for both “Inflammatory response” (such as NFκB1, TIMP1, TNXB, TNFA1P6, RUNX1) and “Extracellular space” (such as THBS4, NOTCH2, ITGBL1) (FIG. 3S 390). Collectively, the inventors' findings show the temporal heterogeneity/dynamics of cellular senescence both inter- and intra-populationally among four major types of mononuclear cells in aged human muscle.

### 2.3 SASP Profiling and Function in Senescent Cells

The inventors mapped the SASP dynamics in ageing muscle. They identified senescence-associated DEGs (Sn-DEG) comparing senescent (Sn) and non-senescent (nSn) cells across the four mononuclear cell populations. A total of 1514 up- and 1576 down-regulated Sn-DEGs were identified in at least one cell type and the majority were cell type specific (FIG. 4A 400), reinforcing the intercellular heterogeneity. By intersecting with the SASP set, 243 of the SASPs were found up-regulated in senescent cells and 78 were commonly shared in at least two cell populations (FIG. 4B 405); in particular, 16 were commonly shared in all four cell types including CXCL8, CXCL2, VCAN, COL12A1, MFAP5, PLAU, CXCL3, FBN1, CD44, CXCL1, SOD2, SLC39A14, MMP3, SERPINE2, ALCAM, and FSTL1 (FIG. 4C 410). Nevertheless, >30% of the SASPs were cell type specific (25, 36, 32, 48 in MuSC, FAP, EC and SMC) (FIG. 4B 405); for example, IGFBP7, NAMPT, CCL2, TGFB, IL7, and ANGPT2 were uniquely up-regulated in senescent MuSCs and THBS1, MAT2A, GALNT2, DPP4, ITGB3, LRPPRC in FAPs (FIG. 4D 415). The findings demonstrate both the commonality and the inter-populational variation in SASP constitutions. When taking a close examination of the MuSC SASPs, the inventors found the SASP ssGSVA score was much higher in senescent vs. non-senescent MuSCs (FIG. 4E 420) and also in aged vs. young MuSCs (FIG. 4F 425). DCN, VCAN, CXCL2, CCL2, CXCL1 were among the top ranked SASPs in both senescent and aged MuSCs (FIGS. 4G 430-4H 435). The induction of DCN, CXCL1, APOD, CCL2, CXCL2, CXCL8, IGFBP6, and EGFR was further validated by RT-qPCR in freshly isolated MuSCs (FIG. 4I 440). The above results illustrate the induction of SASPs and profile their dynamics in senescent mononuclear cells in aged human muscle. Next, to elucidate the function of the SASPs, the inventors mapped SASP-mediated intercellular communication, a total of 810 SASP-mediated ligand (L)-receptor (R) pairs were defined among all cell types by CellChat. Expectedly, SASP-mediated interaction strength was enhanced in the aged vs. young muscle (51.8 vs. 47.2, FIG. 4J 445) along with the overall L-R mediated cell-cell interaction strength (56.8 vs. 52.0), demonstrating SASP function in augmenting cellular communication and altering niche microenvironment in ageing muscle. Consistently, further examination of the receiver-sender cell interactions revealed a global strengthening of SASP (FIG. 4K 450) or all L-R mediated interaction signals between two cell types; for example, the signals to SMCs or ECs from all other types of cells showed a remarkable increase in aged muscle. Interestingly, MuSCs received decreased signals from ECs, FAPs and Pericytes while sending higher levels of signals to BT/NK cells, ECs, MPs and MSMs (FIG. 4K 450). Additionally, the network analysis revealed significantly altered SASP-mediated pathways in scaled information flow (the total sum of communication probability from the inferred SASP network); and a gain of inflammatory SASPmediated pathways (for example by VISFATIN, CD226, ALCAM, and MHC-I signals) was observed in the aged muscle (FIG. 4L 455); ECM-SASP mediated pathways were also altered, for example, increased ITGB2 pathway and decreased PTN pathway was observed (FIG. 4L 455). Additionally, growth factors, such as FGF and IGF-mediated pathways were also changed in aged muscle (FIG. 4L 455). The inventors found that CXCL family (CXCL12, 2, 8, 3 and 1) may act as key SASPs mediating cellular interactions and CXCL-mediated interaction strength and frequency showed significant elevation in aged muscle (FIG. 4M 460). By examining enhanced signalling pairs in the aged group, the inventors identified 54 SASP-mediated L-R pairs with MuSC as either a sender (43), receptor (9) or involved in autocrine interactions (2) (FIGS. 4N 465 and 4O 470). Among the most age-related, increased signals emanating from or received in MuSCs, MPs, SMCs, and FAPs exhibited the highest interaction frequency with MusCs; and CD44 represented a key receptor mediating the interactions (FIG. 4N 465, 4O 470). To further examine the up-regulated SASP-mediated cell interactions in aged MuSCs, analysis of differential communication probability was performed to identify top-ranked L-R pairs emanating from MuSCs. For example, MIF-(CD74+CXCR4) signalling from MuSCs to B/T/NK cells was significantly activated in aged muscle, and MIF-ACKR3 signalling from MuSCs to FAPs, FN1-CD44 signalling from MuSCs to SMCs were also increased (FIG. 4P 475). The above findings show SASP-mediated cellular interactions and demonstrate the key functions of SASPs in modulating muscle microenvironment during muscle ageing.

### 2.4 Maraviroc as a Senotherapeutic for Treating Muscle Degradation

Among all the above-identified SASPs in senescent cells, the inventors observed a robust induction of CCL3, CCL4 and CCL5 along with their receptor CCR5 in aged vs. young MuSCs (FIG. 5A 500) and whole muscle; and the high induction was also confirmed by RT-qPCR in isolated MuSCs from additional human muscle donors (FIG. 5B 505). Moreover, their induction was also found in aged mouse muscles by analysing single-cell RNA-seq data. Unexpectedly, the inventors found that that Maraviroc (MVC), a Ccr5 antagonist is very effective in targeting the Ccl5-Ccr5 axis thus mitigating inflammation in dystrophic mouse muscles. The inventors tested if MVC can be a potential senomorphic for treating muscle ageing by blocking the function of Ccl3, 4 and 5 in mice. In the first high dose short term (HDHT) treatment regime, a high dose (10 mg/kg) of MVC was administered in 18 month old mice intraperitoneally for 3 months (FIG. 5C 510). The MVC treatment led to an evident increase in muscle mass (28.01% increase of TA/body weight) (FIG. 5D 515) and muscle morphology (FIG. 5E 520) compared to the DMSO-treated mice; by H&E staining, the inflammation was also attenuated, and the muscle fiber size was increased (15.50%) (FIG. 5E 520). As a result, muscle function was significantly enhanced which was evidenced by a notable increase in grip strength (15.79%) (FIG. 5F 525) and a tapered grip strength reduction (Δgrip) (19.75%) (FIG. 5G 530). Consistently, when subjected to a treadmill running test, in which the mice were adapted to a treadmill followed by a stepwise increase of running speed until their exhaustion, the MVC-treated mice demonstrated a higher running speed (26.42%) (FIG. 5H 535) and longer running distance (50.00%) (FIG. 5I 540). Overall, the mice were rejuvenated to a much healthier and active state. Furthermore, the number of MuSCs was elevated (3.91% vs. 3.39%) accompanied by decreased macrophages (0.89% vs. 1.09%) but no significant change of FAPs (FIG. 5J 545), indicating MVC treatment blocked the action of SASPs and improved the ageing muscle niche.

The above-uncovered niche impact was further elucidated by single-cell RNA-seq analysis of the 5437 and 5454 mononuclear cells collected from DMSO or MVC-treated mouse muscles. Amongst the total of 12 identified cell populations including Pro-inflammatory MPs (PI-MPs), Anti-inflammatory MPs (AI-MPs), FAPs, EC1, EC2, Tenocytes, B/T/NK cells, SMCs, MuSCs, Neutrophils, MSMs, Pericytes and Schwann cells (FIG. 5K 550) based on normalised gene expression levels and canonical cell type specific markers, the inventors detected a significantly increased population of MuSCs in MVC vs. DMSO group (4.68% vs. 2.65%) accompanied by a reduced population of PI-MPs (1.91% vs. 3.79%) but interestingly not FAPs (27.72% vs. 23.28%) (FIG. 5L 555). Furthermore, the inventors detected significantly decreased levels of cellular senescence in MuSCs (17.6% vs. 20.1%, FIG. 5M 560), FAPs (15.8% vs. 19.3%, FIG. 5N 565), ECs (16.7% vs. 21.9%, FIG. 5O 570) and SMCs (20.1% vs. 31.1%, FIG. 5P 575), which was further supported by the decreased p21 mRNA expression (FIGS. 5M 560, 5N 565, 5O 570, 5P 575). Additionally, cellular crosstalk analysis revealed a global decline of cellular interactions in the MVC-treated muscle. A close examination of SASP-mediated cellular interaction strength showed a decreased interaction strength (FIG. 5Q 580); further examination of the receiver-sender cell interactions also revealed a global decrease of SASP-mediated interaction signals between two cell types except for B/T/NK cell interactions (FIG. 5R 583). For instance, MVC treatment led to reduced interactions mediated by Cxcl pathway among MuSCs, SMCs, neutrophils, and MPs. Ccr5 interactions with its ligands, Ccl3, Ccl4 and Ccl5 were repressed by the MVC treatment (FIG. 5S 586), which was also accompanied by their reduced expression levels in the whole muscle (FIG. 5T 589). To further examine the impact of MVC treatment on MuSCs, bulk RNA-seq was performed on freshly isolated MuSCs from the treated mice. The inventors found that the 231 down-regulated genes (FIG. 5U 592) upon MVC treatment were enriched for SASPs-related terms such as “extracellular space”, “inflammation response” etc. (FIG. 5V 595), indicating repressed SASP expression; this was also confirmed by the Gene Set Enrichment Analysis (GSEA) (FIG. 5W 597). Ccl3, Ccl4, Ccl5 and Ccr5 were among the down-regulated SASPs, which was further confirmed by RT-qPCR (FIG. 5X 599). The above results advantageously demonstrate the use of MVC as a senomorphic for alleviating cellular senescence, improving muscle niche and rejuvenating ageing muscle. To further demonstrate the efficacy of MVC treatment, the inventors tested a low dose (2 mg/kg) long-term (6 months) (LDLT) regime and found the treatment also led to a pronounced restoring effect of muscle morphology, function and niche integrity in aged mice.

### 2.5 Defining TFs Governing Senescence State and SASP Induction in Human Muscle.

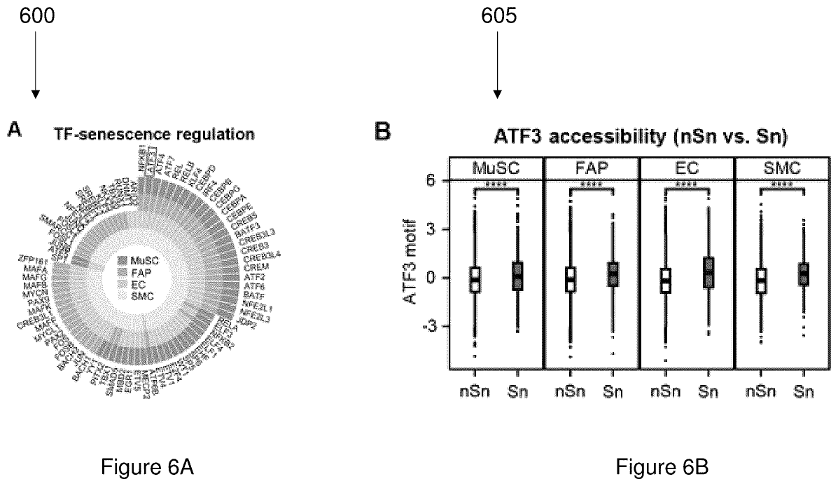

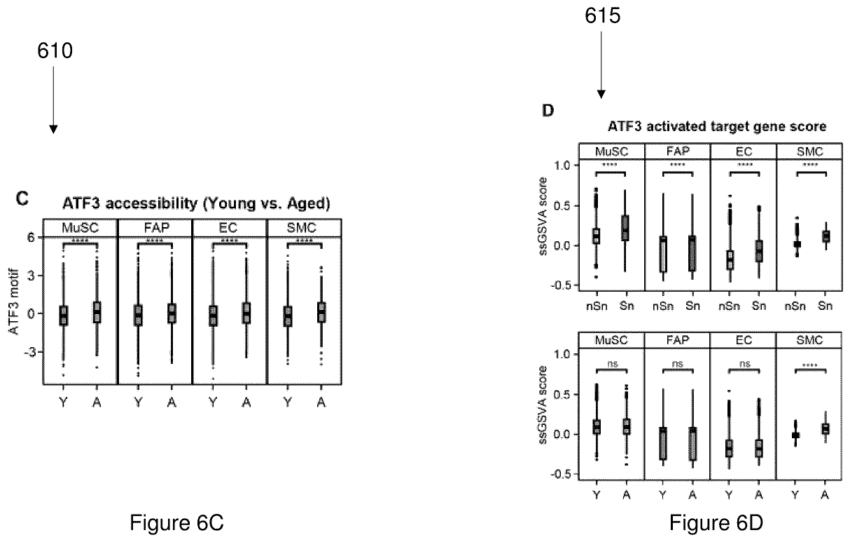

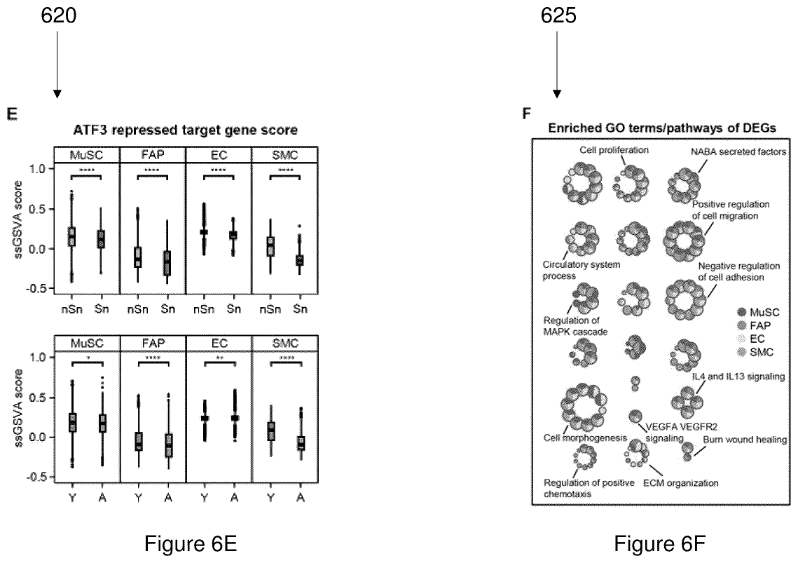

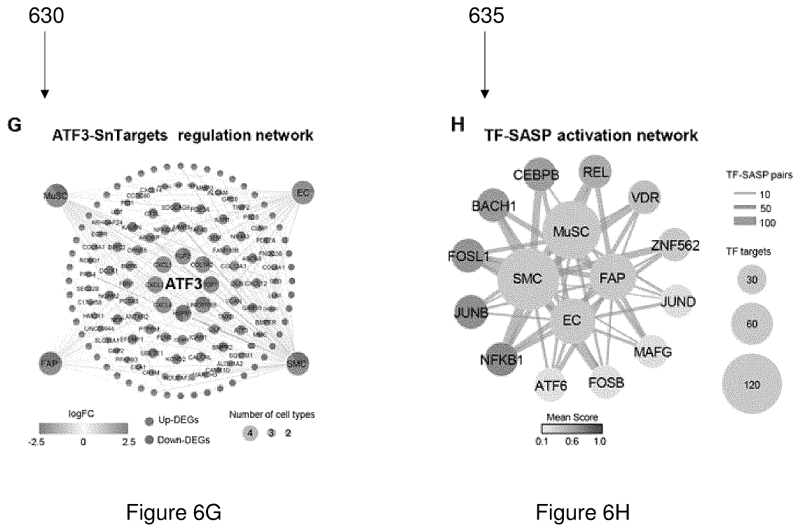

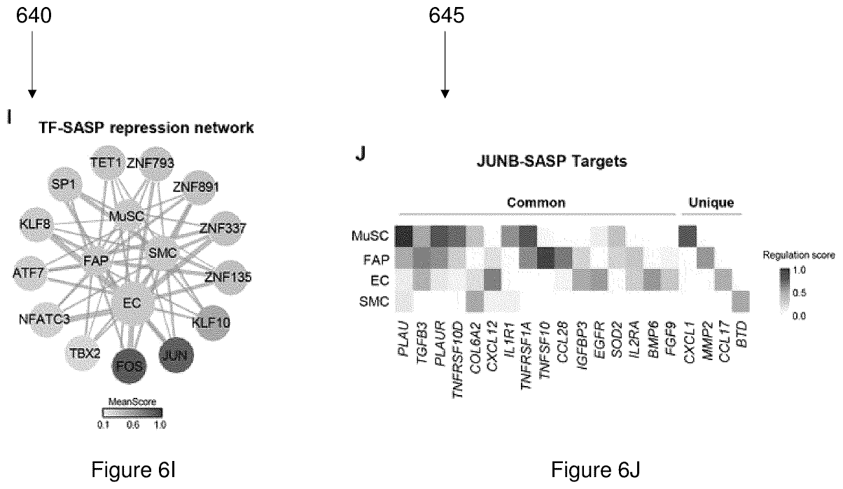

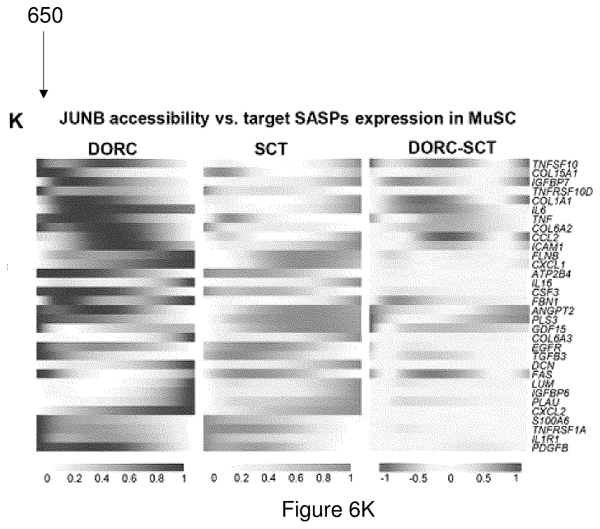

To gain a detailed understanding of the senescence state/SASP regulation in human muscle, the inventors analysed the paired snATAC-seq data which permits accurate identification of potential upstream TF regulators via mapping chromatin accessibility. Key TF regulators of senescent cell state were defined with enriched motifs predicted in the ATAC measurements. 234 TFs were shared in at least two cell types, and 26 were commonly found in all four cell types, including NF-κB family (NF-κB1, REL and RELB), several AP-1 family TFs (ATF2, 3, 4, 6, 7 and BATF3), C/EBP family (C/EBPD, B, G, A) and CREB family (CREB3, 5 and CREB3L3, 4) (FIG. 6A 600). Both NF-κB1 and C/EBPB are evidently key players in the regulation of senescence and SASPs. The inventors also investigated the ATF3 factor. They found that the accessibility of ATF3 binding motifs was increasingly enriched in Sn vs. nSn cells (FIG. 6B 605) and also in aged vs. young cells (FIG. 6C 610) in all four cell types, demonstrating its potential role in regulating senescence and ageing. By integrating the paired snRNA-seq and snATAC-seq data, Functional Inference of Gene Regulation (FigR) analysis was performed to identify target genes that were activated or repressed by ATF3 binding. Transcriptional scores of the ATF3-activated genes were notably elevated in Sn cells (FIG. 6D 615), while the scores of the repressed genes were lower compared to the nSn group (FIG. 6E 620), supporting the notion that ATF3 plays a positive regulatory function in senescent cells. Furthermore, GO functional analysis revealed that ATF3 target genes were enriched for senescence-related terms such as cell proliferation, cell migration, regulation of chemotaxis, regulation of cell adhesion, and ECM organization, etc. (FIG. 6F 625). While displaying similar enrichment terms, differential enrichment patterns were observed in the four cell types. For instance, ATF3 target genes identified in MuSCs were highly enriched for the regulation of the MAPK cascade; target genes in FAPs exhibited a low association with cell morphogenesis, while the targets in SMCs showed limited relevance to extracellular matrix organization. To construct the ATF3 regulatory network, ATF3-regulated senescent DEGs (ATF3-SnTargets) were predicated and many were shared in multiple cell types (FIG. 6G 630). Interestingly, the inventors found that shared target genes (such as genes CXCL8, CXCL2, and CXCL1 shared in all four cell types) were predominantly upregulated in Sn vs. nSn cells (FIG. 6G 630), illustrating ATF3 mainly functions to promote gene expression in senescent cells. The above findings following investigation by the inventors show potential TF regulators of cellular senescence in ageing muscle and highlight ATF3 as a key regulator of senescence in multiple mononuclear cell populations in human muscle. The inventors further elucidated the core TFs governing SASP induction, similarly they constructed TF-SASP regulatory networks and defined potential activating (FIG. 6H 635) and suppressing (FIG. 6I 640) TFs. NF-κB (NF-κB1, REL), and AP-1 family (JUNB, FOSL1, ATF6, FOSB, JUND) TFS were the predominant activators in all four cell types (FIG. 6H 635); AP-1 family, such as JUNB, and FOSL1 were among the most probable TF activators (FIG. 6H 635). The inventors also unexpectedly identified two other AP-1 family TFs, FOS and JUN, as potential suppressors (FIG. 6I 640). To further explore the JUNB-SASP regulation, the inventors assessed JUNB-SASP association using regulation scores and defined a list of common or cell type specific JUNB-activated SASP targets; PLAU was commonly shared in all four cell types while CXCL1, MMP2, CCL17, and BTD were unique to MuSCs, FAPs, ECs and SMCs (FIG. 6J 645). To better assess the role of JUNB in activating SASPs, DORC (domains of regulatory chromatin) analysis was performed to correlate the accessibility of JUNB motif-containing peaks near each SASP target gene with its expression. The difference between the JUNB DORC chromatin accessibility and gene expression along the pseudotime axis was calculated; the inventors found that for most of the JUNB-SASP targets, the chromatin accessibility gain preceded that of the expression change in MuSCs (FIG. 6K 650) as well as the other three cell types. These observations were exemplified on the FBN1 and TNFRSF10D genes during MuSC ageing trajectory; for example, JUNB-related chromatin change of FBN1 was identified as an early event, occurring within the first 47 pseudotime bins, preceding the RNA expression change. The above findings illustrate JUNB as a key upstream TF inducer of SASP production.

### 2.6 JUNB Activates SASP Induction in Senescent MuSCs Via Enhancer Regulation

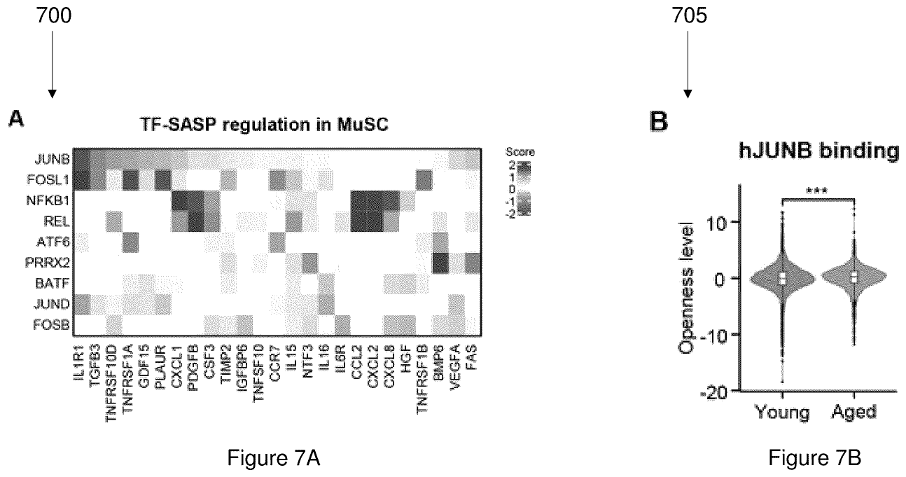

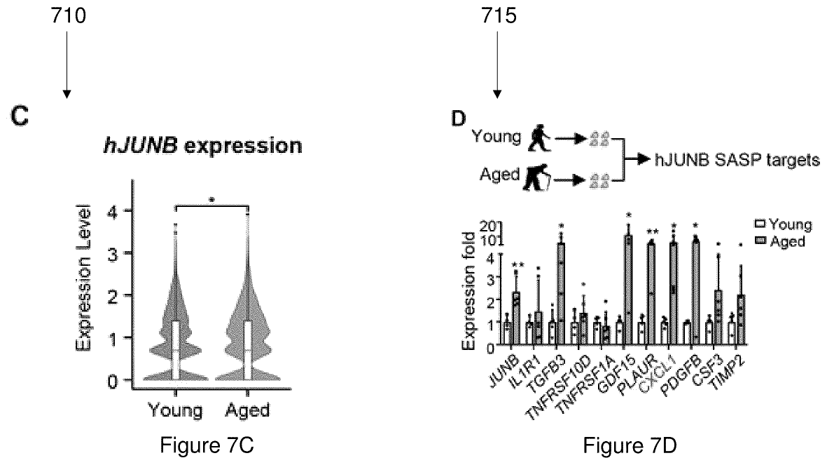

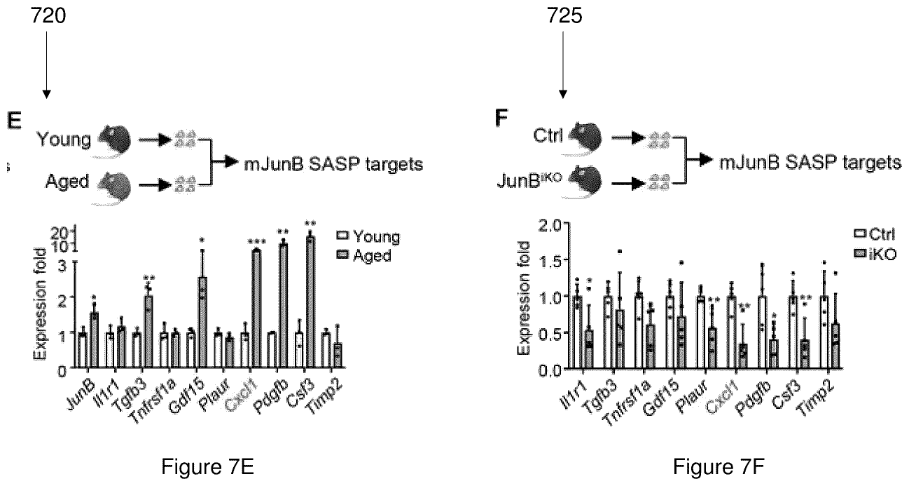

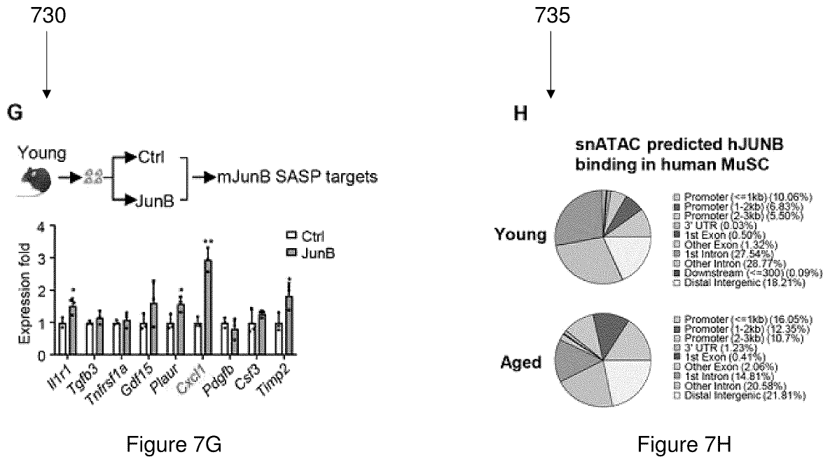

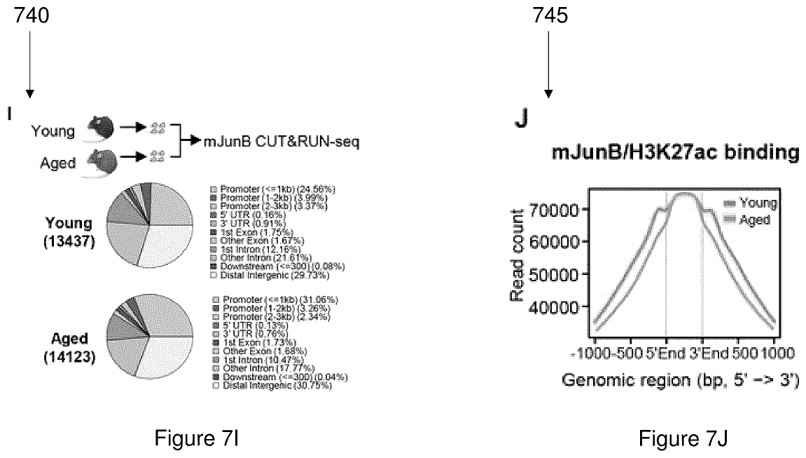

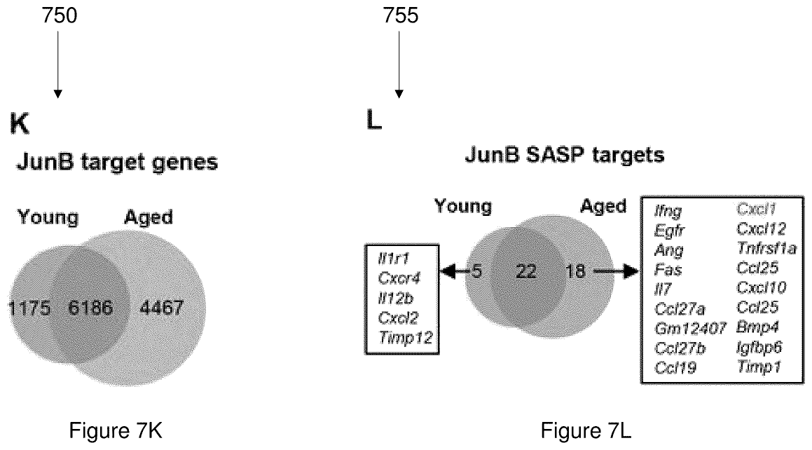

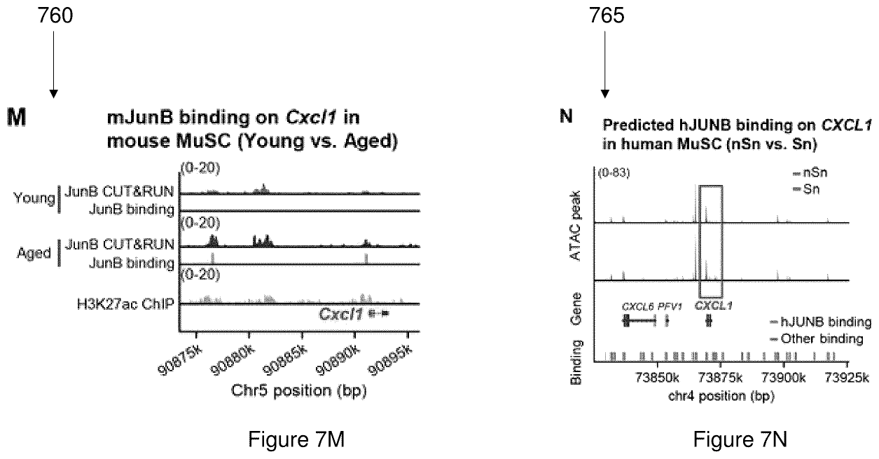

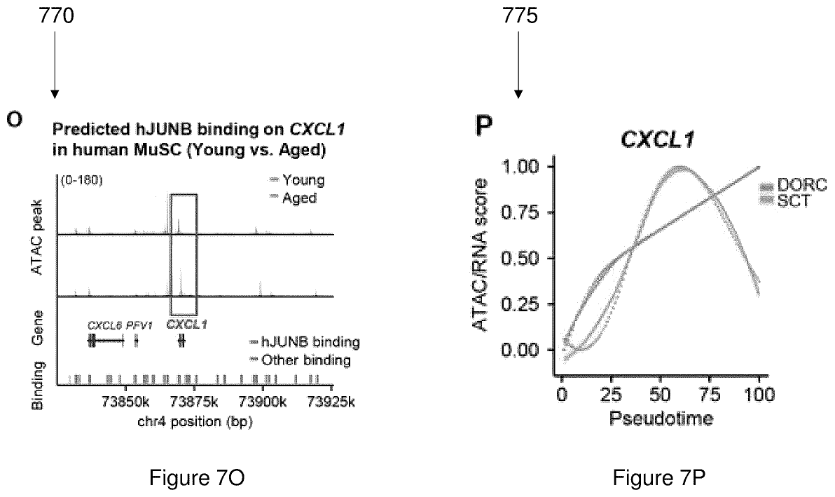

To further elucidate JUNB regulation of SASP induction and senescence in MuSCs, the inventors quantified the TF-SASP association by regulation scores and confirmed that JUNB was a prominent candidate regulator for up-regulated SASPs (FIG. 7A 700). Closer examination revealed that the chromatin openness level of the snATAC-seq showed JUNB binding sites was significantly increased in aged vs. young MuSCs (FIG. 7B 705), supporting the inventors' finding of JUNB to be a key regulatory TF in aged MuSCs. JUNB expression was also significantly elevated in aged MuSCs according to the accompanied scRNA-seq data (FIG. 7C 710) and additionally performed RTqPCR in the isolated human MuSCs (133.35%, FIG. 7D 715). 54 SASPs were predicted to be bound by JUNB including the top-ranked IL1R1, TGFB3, TNFRSF10D, TNFRSF1A, GDF15, PLAUR, CXCL1, PDGFB, CSF3, TIMP2 (FIG. 7A 700); which was also confirmed by RT-qPCR in aged human MuSCs (FIG. 7D 715). NF-κB family regulated a different set of SASPs (FIG. 7A 700). scRNA-seq and scATAC-seq data from mice also shows mouse JunB (mJunB) as an upstream TF regulator of SASP induction in aged mouse MuSCs; consistently, mJunB upregulation was also detected in the MuSCs freshly isolated from aged vs. young mice (58.10%); moreover, the above predicted human SASP targets of human JUNB (hJUNB) were also highly expressed in aged mouse MuSCs (FIG. 7E 720). These results illustrated a role of JUNB in regulating SASP induction in both human and mouse MuSCs. The inventors leveraged an inducible MuSC-specific JunB knockout mouse (JunB-iKO) that was generated by crossing the JunB flox mouse with a Pax7CreER; R26Yfp mouse to further elucidate the regulatory mechanism. MuSCs were isolated from the Ctrl and iKO mice; they found the expression levels of the above-defined mJunB SASP targets were all significantly downregulated (FIG. 7F 725). Moreover, over-expression of mJunB in the MuSCs isolated from young mice induced the expression of some SASP targets including Il1r1, Plaur, Cxcl1 and Timp2 (FIG. 7G 730). Unexpectedly, the above loss or gain of mJunB did not appear to affect the levels of SA-β-GAL and several marker genes such as p16, p19, p21 and p53, indicating mJunB can induce SASP target activation. To further explore how hJUNB or mJunB activates SASP genes, the inventors investigated the snATAC-seq data and found a large percentage of the predicted hJUNB binding was mapped to intergenic and intron regions (56.31% in young and 35.59% in aged) in MuSCs (FIG. 7H 735), indicating hJUNB may regulate gene expression primarily through enhancer binding. To further dissect the regulatory mechanism, CUT&RUN-seq was conducted to map mJunB binding in MuSCs from young and aged mice to define a total of 13,437 and 14,123 mJunB binding events. Consistent with the above, a large percentage of mJunB binding peaks were mapped to intergenic and intron regions (63.50% in young and 58.99% in aged) in MuSCs (FIG. 7I 740), and more than 60% of the binding sites were >3 kb distal to the TSSs while a small portion of promoter binding (<=3 kb) was observed (36.66% in aged and 31.92% in young). By intersecting with the H3K27ac CUT&RUN-seq data performed in mouse MuSCs, the inventors found that most mJunB-binding sites were in active enhancer regions (FIG. 7J 745), and its enhancer binding evidently increased in aged vs. young MuSCs (6,648 vs. 6,042). Based on promoter and enhancer binding, the inventors identified a total of 7,361 mJunB target genes in young and 10,653 in aged MuSCs (FIG. 7K 750), which were enriched for GO functions related to “protein modification” and “catabolic process”. To elucidate how mJunB activates SASP transcription, they defined a number of the SASP targets that were directly bound by mJunB, and the number increased in aged (40) vs. young (27) MuSCs (FIG. 7L 755). mJunB binding mostly resided in the enhancer regions of these SASP targets. Notably, Cxcl1 was shown as a prominent target in both human and mouse (FIG. 7D 715-7E 720) and mJunB bound to both the promoter and an enhancer region of Cxcl1 in aged mouse MuSCs (FIG. 7M 760). In human MuSCs, the predicted hJUNB binding on CXCL1 resided in a downstream region and showed a significantly elevated chromatin openness level in senescent (FIG. 7N 765) and aged human MuSCs (FIG. 7O 770). Furthermore, DORC analysis on CXCL1 locus illustrated the chromatin accessibility gain preceded the transcriptional induction, indicating the activating role of hJUNB in CXCL1 transcription (FIG. 7P 775). The above results demonstrate the key role of hJUNB/mJunB in governing SASP induction in aged MuSCs.

### 2.7 JUNB Inhibition Rejuvenates Senescent Human Fibroblasts

To further test the role of JUNB as a general TF regulator of SASP induction, the inventors used human fibroblast cell line IMR-90 to induce senescence by Etoposide (ETO) treatment (FIG. 8A 800). The expression of JUNB was highly induced along with the levels of SA-β-GAL and senescent markers P14, P16, P19 and P21 (FIG. 8A 800, 8B 805); the majority of the SASP targets identified in MuSCs were also induced (FIG. 8C 810). Knocking down JUNB by siRNA oligos before the ETO treatment (FIG. 8D 815) significantly delayed the degree of senescence (FIG. 8E 820-8F 825) and reduced the expression of CXCL1 (FIG. 8G 830). To strengthen the above findings, replicative senescence was also established in the IMR-90 cells (FIG. 8A 800); evident senescence was observed after the cells (P1) were passaged for 10 generations (P10) (FIG. 8A 800, 8B 805, 8C 810). Notably, knocking down JUNB in the P10 cells reversed the senescence (FIG. 8H 835, 8I 840, 8J 845) and diminished CXCL1 induction (FIG. 8K 850). Next, the IMR-90 fibroblasts were treated with T5224, a small molecule inhibitor of c-Fos/AP-1, to block the function of JUNB. Consistently, the treatment led to delayed senescence and decreased CXCL1 expression in both ETO (FIGS. 8L 855, 8M 860, 8O 860) and replication-induced senescence (FIGS. 8P 869, 8S 878) without impacting JUNB expression (FIGS. 8L 855, 8P 869). These results demonstrate illustrate the advantages of pharmacological targeting of JUNB as an effective senomorphic approach. To further dissect the regulatory mechanism of JUNB in senescent IMR-90 cells CUT&RUN-seq was conducted in non-senescent Ctrl and ETO-induced senescent cells (FIG. 8T 881). A total of 58,718 and 72,714 JUNB binding events were identified in the cells. Notably, a high percentage of JUNB binding was mapped to intergenic and intron regions (86.14% in ETO and 86.53% in Ctrl) while only a small portion on the promoter regions (<=1 kb) was found (3.99% in ETO and 3.95% in Ctrl) (FIG. 8U 884). The inventors found that most JUNB-binding sites resided in active enhancer regions and the number increased in ETO-treated cells (60,860 vs. 50,851) (FIG. 8V 887). Based on promoter and enhancer binding, they identified a total of 11,783 JUNB target genes in Ctrl and 14,806 in ETO IMR-90 cells (FIG. 8W 890). GO analysis of JUNB target genes revealed enrichment of several shared functional categories, including “cellular component disassembly,” “proteasome-mediated ubiquitin-dependent protein catabolic process,” and “chromosome segregation”, etc. Notably, the genes bound by JUNB specifically in ETO cells demonstrated enrichment in senescence-related terms, such as “negative regulation of cell migration”, “regulation of Wnt signalling pathway” and “cytokinesis”. Next, the inventors identified the directly bound SASP targets of JUNB. A total of 71 and 73 targets were found in the Ctrl and ETO cells with the majority (63) shared (FIG. 8X 893). JUNB binding was predominantly found in the enhancer regions of these targets. Furthermore, the inventors analysed the enhancer-promoter looping and discovered that JUNB primarily bound on the enhancers involved in E-P looping (FIG. 8Y 896), indicating JUNB orchestrating the E-P interaction to modulate SASP transcription. For example, on the CXCL1 locus, JUNB bound to an enhancer region 11 kb downstream which looped with the promoter in both Ctrl and ETO cells, but a second binding was induced at a different enhancer 5 kb downstream after ETO treatment co-currently with the formation of an evident E-P looping (FIG. 8Z 899), illustrating that JUNB binding may orchestrate E-P loop formation to activate CXCL1 transcription. Beneficially, the inventors' findings demonstrate the key role of JUNB in regulating senescence and its potential as a senotherapeutic target.

### Example 3—Discussion

The inventors conducted multiomics mapping of cellular senescence atlas in ageing human muscle. Using a USS scoring system, they mapped cellular senescence in mononucleated cells in ageing muscle and uncovered commonality and heterogeneity of a senescence state among different cells. Knowing SASPs are the main determinant of senescence state and function, they advantageously further defined SASP composition and revealed the heterogeneity and dynamics in SASP constitution and expression. Further, they dissected key TFs governing cellular senescence and SASP production and illustrated the key role of AP-1 family TF such as ATF3 and JUNB in senescence/SASP regulation. The inventors, in the examples, have shown the prevalence of senescence in multiple mononuclear cells and pinpoints the presence of MuSC senescence and its potential function in altering niche microenvironment. More importantly, the mapping led to identification of MVC as a potential senotherapeutic approach to treat muscle degradation and sarcopenia, for example delay sarcopenia progress and rejuvenate ageing mice.

Meanwhile, the inventors have shown that JUNB could be a general regulator of cellular senescence and targeting JUNB as an effective senotherapeutic approach to reverse senescence.

The inventors have provided comprehensive mapping and characterisation of cellular senescence in ageing human muscle. Advantageously, this multiomics mapping harnessing simultaneous single-nucleus RNA-seq/ATAC-seq enables dissection of transcriptomic and epigenomic features of senescence in the same cells. By removing the terminally differentiated, post-mitotic myofibers from the analysis, the inventors were able to focus on the mononucleated cells in the niche that are prone to senescence permitting an in-depth examination of senescence in MuSCs for the first time. Moreover, the inventors have provided the first senescence blueprint on human skeletal muscles. The inventors' mapping is largely facilitated by creating a USS scoring method that combines known senescent gene sets, thus providing a useful and effective tool for defining the wide spectrum of senescence.

Obtaining multiomics data from senescent cells to provide a senescence atlas will advantageously provide a holistic understanding of the transcriptomic and epigenomic heterogeneity of senescent cells in ageing human muscles. This will contribute to a better grasp of the biological functions of these cells and facilitate the identification of new markers and therapeutic strategies for alleviating sarcopenia and promoting healthy ageing. The inventors' mapping uncovered wide variations of senescence at both inter- and intra-populational levels in ageing muscle. Pseudotime analysis uncovered a temporal dynamic of senescence within each of the four examined cell types; senescent MusCs clearly diverged into two different fates at the intra-populational level and senescence also showed marked different temporal patterns within FAPs, ECs and SMCs. The heterogeneity is also largely reflected by the SASP constitutions-unique compositions of SASP factors were defined in each cell population. The inventors were able to uncover shared features of senescence among the cells and a panel of common SASPs were found (FIG. 4A 400-4P 475); these SASPs constitute promising clinical biomarkers for assessing the burden of senescence in ageing muscle tissue and also can serve as senomorphic targets for simultaneously blocking SASP action in multiple cells. SASP factors are the major mediators of the non-cell-autonomous effects of senescent cells through their paracrine effect in dispersing senescence and altering the niche microenvironment. The cell-cell interaction analysis sheds light on the complex SASPmediated cellular interactions. The inventors have shown that inflammatory SASP-mediated pathways were largely enhanced in aged muscle (FIG. 4L 455), illustrating the key role of SASPs in inflammation occurring in aged skeletal muscle tissue. These defined interactions such as CXCL-mediated interactions occurring among multiple cell types thus represent targets for senomorphic design. The analysis of MuSC-centered cellular interactions by the inventors demonstrated that MuSCs can actively modulate the niche through their secretory function. In aged human muscle, the inventors' finding defined many signalling pairs through which MuSCs communicate with other cell populations via secreting SASPs (FIGS. 4N 465, 4O 470, 4P 475). Further, the heterogeneous nature of SASPs/senescence can also arise from the inducers and regulators of SASPs. By harnessing the snATAC-seq dataset, the inventors gained the first comprehensive mapping of the upstream TF regulators of both senescence state and SASPs in ageing human muscle. Along with NF-κB, AP-1 family appeared as a dominant transcriptional activator of senescence/SASPs in multiple cell populations. The inventors' findings above show a key role of ATF3 in governing senescent state in ageing muscle via its extensive regulatory targets/networks. In terms of SASP regulation, the inventors identified JUNB, also an AP-1 factor, as a prominent activator of SASP transcription in multiple cells and accordingly conducted an in-depth mechanistic dissection of how JUNB activates SASPs in MuSCs. The inventors' findings demonstrate that JUNB activates SASP activation in both human and mouse MuSCs. The direct JUNB SASP targets were identified and CXCL1 appeared to be a prominent target shared in both human and mouse. Furthermore, their findings also uncovered that JUNB regulation of SASP transcription is through facilitating enhancer-promoter looping formation thus demonstrating AP-1 TFs pioneer the senescence enhancer landscape and drive the transcriptional program in human lung fibroblasts undergoing oncogene-induced senescence. The inventors have beneficially shown the positive role of JUNB in regulating SASP transcription potentially via facilitating E-P looping. Both siRNA inhibition and pharmacological blocking of JUNB action repressed the senescence program and SASP production. The inventors' findings highlight the transcriptomic and epigenomic variations at intra and inter-populational levels in ageing human muscle.

The claimed method allows for the identification of druggable targets for senotherapeutics to ameliorate muscle degradation and sarcopenia and enhance healthy muscle ageing. The inventors found that systemic delivery of MVC that blocked CCL3, 4, 5 action significantly enhanced muscle performance, delayed muscle ageing and led to remarkable rejuvenating effects; the effect was significant under the HDST, LDLT treatment schemes. The treatment diminished senescence in multiple cells and reversed the deregulated ageing muscle niche, demonstrating the feasibility of pharmacological SASP inhibition as a senomorphic therapy for sarcopenia and potentially other conditions involving CCLs producing senescent cells. The inventors' findings are the first demonstrating the novel utility of MVC for ameliorating muscle ageing. They also demonstrated the anti-inflammatory effect of MVC treatment on DMD muscle, thereby providing a pharmacologic intervention that concurrently targets both cellular senescence and inflammation, the two associated hallmarks of organismal ageing.

1. A method for identifying senotherapeutic targets, comprising the steps of:
obtaining multiomics data from senescent cells to provide a senescence atlas,
utilising the senescence atlas to identify molecular markers associated with cellular senescence,
comparing the identified molecular markers with senescence-associated secretory phenotypes (SASPs) and senescence biomarkers, and
identifying potential senotherapeutic targets involved in tissue degradation.

2. The method of claim 1, wherein the molecular markers include one or more of genes, proteins, epigenetic modifications, or secretory factors associated with senescence.

3. The method of claim 1, wherein the multiomics data includes one or more transcriptomic, epigenomic, or proteomic profiles.

4. The method of claim 1, wherein the multiomics data is obtained using single-nucleus sequencing or other single-cell omics technologies.

5. The method of claim 1, further comprising a step of validating the identified potential senotherapeutic targets by assessing the role in cellular senescence in vitro or in vivo.

6. The method of claim 1, further comprising a step of determining the effects of targeting the potential senotherapeutic targets on cellular senescence.

7. The method of claim 1, wherein the potential senotherapeutic targets include genes, proteins, or signalling pathways involved in one or more of cellular senescence, inflammation, muscular degradation, sarcopenia, or age-related diseases.

8. The method of claim 1, wherein the senescence atlas is used to compare senescent and non-senescent cell profiles to identify specific alterations associated with senescence.

9. A method of treating muscle degradation in a subject, the method comprising administering to the subject a composition comprising a therapeutically effective amount of a compound of Formula (I), or a pharmaceutically acceptable salt, solvate, or hydrate thereof:

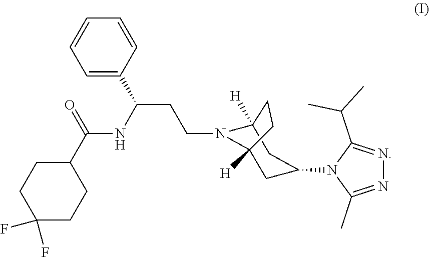 [SMILES: Cc1nnc(C(C)C)n1[C@@H]1C[C@H]2CC[C@@H](C1)N2CC[C@H](NC(=O)C1CCC(F)(F)CC1)c1ccccc1]

10. The method of claim 9, wherein the composition reduces or delays cellular senescence in the subject.

11. The method of claim 9, wherein the composition targets cellular senescence mechanisms to mitigate muscle degradation and improve muscle function in the subject.

12. The method of claim 9, wherein the composition targets senescence-associated secretory phenotypes (SASPs) to mitigate muscle degradation and improve muscle function in the subject.

13. The method of claim 9, wherein the composition targets one or more senotherapeutic targets.

14. The method of claim 9, wherein the composition targets one or more senotherapeutic targets including genes, proteins, or signalling pathways involved in one or more of cellular senescence, inflammation, muscular degradation, sarcopenia, neurodegenerative diseases, cardiovascular ageing, or age-related diseases.

15. The method of claim 9, wherein the therapeutically effective amount of the compound of Formula (I) administered is determined based on an assessment of degree of the muscle degradation in the subject and a severity of cellular senescence in the subject.

16. The method of claim 9, wherein the muscle degradation is associated with sarcopenia, muscle wasting disorders, age-related muscular weakness, or a combination thereof.

17. The method of claim 9, wherein the muscle degradation is associated with sarcopenia.

18. The method of claim 9, wherein the composition is formulated for administration in the subject via oral, injectable, or topical routes.

19. The method of claim 9, further comprising administering the composition in combination with one or more therapeutic agents or supplementary treatments targeting muscle regeneration or senescence.

20. The method of claim 19, wherein the therapeutic agents include muscle growth stimulants, anti-inflammatory agents, or other senotherapeutic compounds, or a combination thereof.

21. The use of a compound of Formula (I), or a pharmaceutically acceptable salt, solvate, or hydrate thereof, in the manufacture of a medicament for the treatment of muscle degradation in a subject:

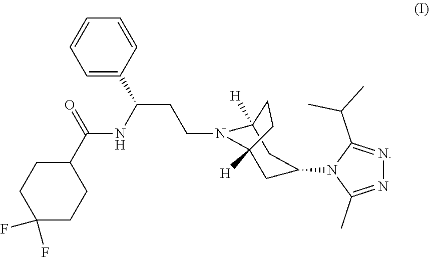 [SMILES: Cc1nnc(C(C)C)n1[C@@H]1C[C@H]2CC[C@@H](C1)N2CC[C@H](NC(=O)C1CCC(F)(F)CC1)c1ccccc1]

22. The use of claim 21, wherein the medicament reduces or delays cellular senescence.

23. The use of claim 21, wherein a therapeutically effective amount of the medicament is administered to mitigate muscle degradation and improve muscle function.

24. The use of claim 21, wherein the medicament is formulated to modulate the function of senescence-associated secretory phenotypes (SASPs) in muscle cells.

25. The use of claim 21, wherein the medicament targets one or more senotherapeutic targets including genes, proteins, or signalling pathways involved in one or more of cellular senescence, inflammation, muscular degradation, sarcopenia, neurodegenerative diseases, cardiovascular ageing, or age-related diseases.

26. The use of claim 21, wherein the medicament is formulated as a dosage form selected from the group consisting of tablets, capsules, and powders.

27. The use of claim 21, wherein the medicament is formulated to deliver the compound of Formula (I) in an extended-release or controlled-release dosage form.

28. The use of claim 21, wherein the muscle degradation is associated with sarcopenia, muscle wasting disorders, age-related muscular weakness, or a combination thereof.

29. The use of claim 21, wherein the muscle degradation is associated with sarcopenia.

30. The use of claim 21, wherein the medicament further comprises one or more pharmaceutically acceptable carriers or excipients.

31. A method of treating muscle degradation in a subject, the method comprising administering to the subject a composition comprising a therapeutically effective amount of a compound of Formula (II), or a pharmaceutically acceptable salt, solvate, or hydrate thereof:

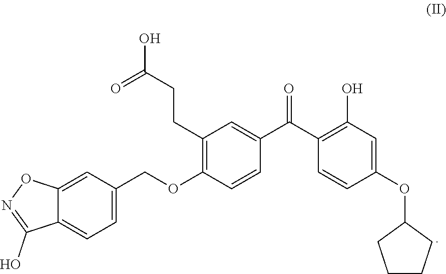 [SMILES: O=C(O)CCc1cc(C(=O)c2ccc(OC3CCCC3)cc2O)ccc1OCc1ccc2c(O)noc2c1]

32. The method of claim 31, wherein the composition targets cellular senescence mechanisms to mitigate muscle degradation and improve muscle function in the subject.

33. The method of claim 31, wherein the composition targets c-Fos/activator protein (AP)-1 family.

34. The method of claim 31, wherein the composition targets one or more of JUNB, FOSL1, ATF6, FOSB, and JUND, resulting in delayed senescence.

35. The method of claim 31, wherein the composition targets JUNB to delay cellular senescence.

36. The method of claim 31, wherein the composition is administered in combination with a therapeutically effective amount of a compound of Formula (I), or a pharmaceutically acceptable salt, solvate, or hydrate thereof:

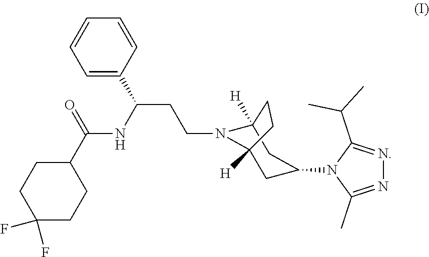 [SMILES: Cc1nnc(C(C)C)n1[C@@H]1C[C@H]2CC[C@@H](C1)N2CC[C@H](NC(=O)C1CCC(F)(F)CC1)c1ccccc1]

37. The method of claim 31, wherein the therapeutically effective amount of the compound of Formula (II) administered is determined based on an assessment of degree of the muscle degradation in the subject and a severity of cellular senescence in the subject.

38. The method of claim 31, wherein the muscle degradation is associated with sarcopenia, muscle wasting disorders, age-related muscular weakness, or a combination thereof.

39. The method of claim 31, wherein the muscle degradation is associated with sarcopenia.

40. The method of claim 31, wherein the composition is formulated for administration in the subject via oral, injectable, or topical routes.

41. The method of claim 31, further comprising administering the composition in combination with one or more therapeutic agents or supplementary treatments targeting muscle regeneration or senescence, including muscle growth stimulants, anti-inflammatory agents, other senotherapeutic compounds, or a combination thereof.

42. The use of a compound of Formula (II), or a pharmaceutically acceptable salt, solvate, or hydrate thereof, in the manufacture of a medicament for the treatment of muscle degradation in a subject:

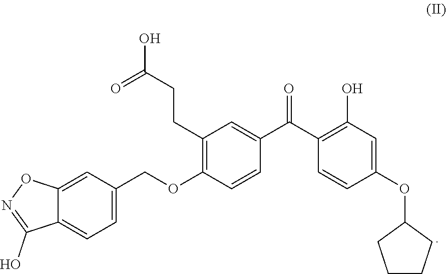 [SMILES: O=C(O)CCc1cc(C(=O)c2ccc(OC3CCCC3)cc2O)ccc1OCc1ccc2c(O)noc2c1]

43. The use of claim 42, wherein the medicament reduces or delays cellular senescence.

44. The use of claim 42, wherein a therapeutically effective amount of the medicament is administered to mitigate muscle degradation and improve muscle function.

45. The use of claim 42, wherein the medicament targets c-Fos/activator protein (AP)-1 family.

46. The use of claim 42, wherein the medicament is formulated to modulate JUNB, FOSL1, ATF6, FOSB, and JUND in muscle cells.

47. The use of claim 42, wherein the medicament targets JUNB to delay cellular senescence.

48. The use of claim 42, wherein the medicament targets genes, proteins, or signalling pathways involved in one or more of cellular senescence, inflammation, muscular degradation, sarcopenia, neurodegenerative diseases, cardiovascular ageing, or age-related diseases.

49. The use of claim 42, wherein the medicament further comprises a therapeutically effective amount of a compound of Formula (I), or a pharmaceutically acceptable salt, solvate, or hydrate thereof:

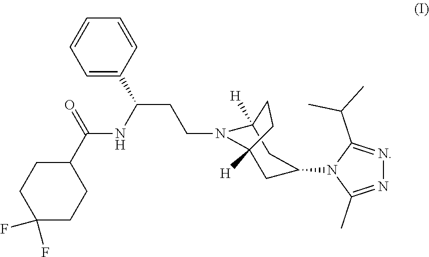 [SMILES: Cc1nnc(C(C)C)n1[C@@H]1C[C@H]2CC[C@@H](C1)N2CC[C@H](NC(=O)C1CCC(F)(F)CC1)c1ccccc1]

50. The use of claim 42, wherein the medicament is formulated as a dosage form selected from the group consisting of tablets, capsules, and powders.

51. The use of claim 42, wherein the medicament is formulated to deliver the compound of Formula (II) in an extended-release or controlled-release dosage form.

52. The use of claim 42, wherein the muscle degradation is associated with sarcopenia, muscle wasting disorders, age-related muscular weakness, or a combination thereof.

53. The use of claim 42, wherein the muscle degradation is associated with sarcopenia.

54. The use of claim 42, wherein the medicament further comprises one or more pharmaceutically acceptable carriers or excipients.
